(19) United States
(12) Patent Application Publication
(10) Pub. No.: US 2005/0107387 A1
(43) Pub. Date: May 19, 2005

(54) Compositions of a cyclooxygenase-2 selective inhibitor and a peroxisome proliferator activated receptor agonist for the treatment of ischemic mediated central nervous system disorders

(75) Inventors: Philip Needleman, Creve Coeur, MO (US); Mark G. Obukowicz, Kirkwood, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation, St. Louis, MO (US)

(21) Appl. No.: 10/844,269

(22) Filed: May 12, 2004

Related U.S. Application Data
(60) Provisional application No. 60/470,240, filed on May 13, 2003.

(51) Int. Cl. A61K 31/50; A61K 31/425; A61K 31/415; A61K 31/4439; A61K 31/426; A61K 31/365; A61K 31/18

(52) U.S. Cl. 514/247; 514/340; 514/369; 514/406; 514/471; 514/602; 514/374

(57) ABSTRACT

The present invention provides compositions and methods for the treatment of ischemic mediated central nervous system disorders. More particularly, the invention provides a combination therapy for the treatment of a central nervous system ischemic mediated disorder comprising the administration to a subject of a peroxisome proliferator activated receptor agonist in combination with a cyclooxygenase-2 selective inhibitor.

### CROSS REFERENCE TO RELATED APPLICATION

This application claims priority from Provisional Application Ser. No. 60/470,240 filed on May 13, 2003, which is hereby incorporated by reference in its entirety.

### FIELD OF THE INVENTION

The present invention provides compositions and methods for the treatment of reduced blood flow to the central nervous system. More particularly, the invention is directed toward a combination therapy for the treatment or prevention of ischemic-mediated central nervous system disorders, including ischemic stroke, comprising the administration to a subject of a peroxisome proliferator activated receptor agonist in combination with a cyclooxygenase-2 selective inhibitor.

### BACKGROUND OF THE INVENTION

The continued increase in the incidence of ischemic-mediated central nervous system damage, including ischemic stroke, provides compelling evidence that there is a continuing need for better treatment strategies. Stroke, for example, is consistently the second or the third leading cause of death annually and the leading producer of disability among adults in the United States and western countries. Moreover, roughly 10% of patients with stroke become heavily handicapped, often needing attendant care.

Within the 1990's decade, the pathology underlying ischemic-mediated central nervous system injury was elucidated. Generally speaking, the normal amount of perfusion to brain gray matter is 60 to 70 mL/100 g of brain tissue/min. Death of central nervous system cells typically occurs only when the flow of blood falls below a certain level (approximately 8-10 mL/100 g of brain tissue/min) while at slightly higher levels the tissue remains alive but not able to function. For example, most strokes culminate in a core area of cell death (infarction) in which blood flow is so drastically reduced that the cells usually cannot recover. This threshold seems to occur when cerebral blood flow is 20 percent of normal or less. Without neuroprotective agents, nerve cells facing 80 to 100 percent ischemia will be irreversibly damaged within a few minutes. Surrounding the ischemic core is another area of tissue called the “ischemic penumbra” or “transitional zone” in which cerebral blood flow is between 20 and 50 percent of normal. Cells in this area are endangered, but not yet irreversibly damaged. Thus in the acute stroke, the affected central core brain tissue may die while the more peripheral tissues remain alive for many years after the initial insult, depending on the amount of blood the brain tissue receives.

At the cellular level, if left untreated, rapidly within the core infarction, and over time within the ischemic penumbra, brain or spinal cell injury and death progress in stepwise manner. Without adequate blood supply, brain or spinal cells lose their ability to produce energy, particularly adenosine triphosphate (ATP). When this energy failure occurs, brain or spinal cells become damaged and will die if critical thresholds are reached. Immediate cell death within the ischemic core is typically necrotic, while cell death in the penumbra may be either necrotic or apoptotic. It is believed that there are an immense number of mechanisms at work causing brain or spinal cell damage and death following energy failure. Each of these mechanisms represents a potential route for intervention. One of the ways brain cells respond to energy failure is by elevating the concentration of intracellular calcium. Worsening this and driving the concentrations to dangerous levels is the process of excitotoxicity, in which brain cells release excessive amounts of glutamate, a neurotransmitter. This stimulates chemical and electrical activities in receptors on other brain cells, which leads to the degradation and destruction of vital cellular structures. Brain cells ultimately die as a result of the actions of calcium-activated proteases (enzymes which digest cell proteins), lipases (enzymes which digest cell membranes) and free radicals formed as a result of the ischemic cascade.

Several studies indicate that cyclooxygenase-2 is involved in the inflammatory component of the ischemic cascade. Cyclooxygenase-2 expression is known to be induced in the central nervous system following ischemic injury. In one study, it was shown that treatment with a cyclooxygenase-2 selective inhibitor reduced infarct volume in mice subjected to ischemic brain injury (Nagayama et al., (1999) J. Cereb. Blood Flow Metab. 19(11):1213-19). A similar study showed that cyclooxygenase-2 deficient mice have a significant reduction in brain injury produced by occlusion of the middle cerebral artery when compared to mice that express cyclooxygenase-2 (Iadecola et al., (2001) PNAS 98:1294-1299). Another study demonstrated that treatment with cyclooxygenase-2 selective inhibitor results in improved behavioral deficits induced by reversible spinal ischemia in rabbits (Lapchak et al., (2001) Stroke 32(5):1220-1230).

No drug therapy has yet been proven completely effective in preventing brain damage from cerebral ischemia. Interventions have been directed toward salvaging the ischemic penumbra and reducing its size. Restoration of blood flow is the first step toward rescuing the tissue within the penumbra. Therefore, timely recanalization of an occluded vessel to restore perfusion in both the penumbra and in the ischemic core is one treatment option employed. Partial recanalization also markedly reduces the size of the penumbra as well. Moreover, intravenous tissue plasminogen activator and other thrombolytic agents have been shown to have clinical benefit if they are administered within a few hours of symptom onset. Beyond this narrow time window, however, the likelihood of beneficial effects is reduced and hemorrhagic complications related to thrombolytic agents become excessive, seriously compromising their therapeutic value. Hypothermia decreases the size of the ischemic insult in both anecdotal clinical and laboratory reports. In addition, a wide variety of agents have been shown to reduce infarct volume in animal models. These agents include pharmacologic interventions that involve thrombolysis, calcium channel blockade, and cell membrane receptor antagonism have been studied and have been found to be beneficial in animal cortical stroke models. But there is a continuing need for improved treatment regimes following ischemic-mediated central nervous system injury.

### SUMMARY OF THE INVENTION

Among the several aspects of the invention is provided a method for the treatment of ischemic mediated central nervous system disorders in a subject. The method comprises administering to the subject a cyclooxygenase-2 selective inhibitor or a pharmaceutically acceptable salt or a prodrug thereof in combination with a peroxisome proliferator activated receptor agonist or pharmaceutically acceptable salt or prodrug thereof.

In one embodiment, the cyclooxygenase-2 selective inhibitor is a member of the chromene class of compounds. For example, the chromene compound may be a compound of the formula 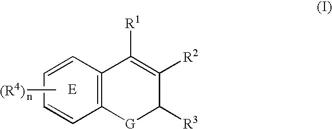
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or wherein R⁴ together with the carbon atoms to which it is attached and the remainder of ring E forms a naphthyl radical.

In another embodiment, the cyclooxygenase-2 selective inhibitor or a pharmaceutically acceptable salt or a prodrug thereof comprises a compound of the formula 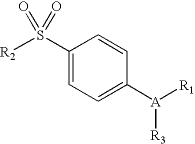
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

In yet another embodiment, the peroxisome proliferator activated receptor agonist is a thiazolidinedione. In one alternative of this embodiment, the thiazolidinedione is selected from the group consisting of ciglitazone, troglitazone, pioglitazone, englitazone, rosiglitazone, and darglitazone.

In still another embodiment, the peroxisome proliferator activated receptor agonist is a member of the fibrate class of compounds. In one alternative of this embodiment, the fibrate compound is selected from the group consisting of clofibrate, fenofibrate, and bezafibrate.

In a further embodiment, the peroxisome proliferator activated receptor agonist is a fatty acid analogue. In one alternative of this embodiment, the fatty acid analogue is tetradecylthioacetic acid or tetradecylselenoacetic acid.

Other aspects of the invention are described in more detail below.

Abbreviations and Definitions

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxycarbonyl” is a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkylamino” is an amino group that has been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” is a radical containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radicals. More preferred are “lower aminoalkyl” radicals. Examples of such radicals include aminomethyl, aminoethyl, and the like.

The term “aminocarbonyl” is an amide group of the formula —C(═O)NH2.

The term “aralkoxy” is an aralkyl radical attached through an oxygen atom to other radicals.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “aralkylamino” is an aralkyl radical attached through an amino nitrogen atom to other radicals. The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aralkylthio” is an aralkyl radical attached to a sulfur atom.

The term “aralkylthioalkyl” is an aralkylthio radical attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl”, alone or in combination, is a carbocyclic aromatic system containing one, two or three rings wherein such rings may be attached together in a pendent manner or may be fused. The term “aryl” includes aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “arylamino” is an amino group, which has been substituted with one or two aryl radicals, such as N-phenylamino. The “arylamino” radicals may be further substituted on the aryl ring portion of the radical.

The term “aryloxyalkyl” is a radical having an aryl radical attached to an alkyl radical through a divalent oxygen atom.

The term “arylthioalkyl” is a radical having an aryl radical attached to an alkyl radical through a divalent sulfur atom.

The term “carbonyl”, whether used alone or with other terms, such as “alkoxycarbonyl”, is —(C═O)—.

The terms “carboxy” or “carboxyl”, whether used alone or with other terms, such as “carboxyalkyl”, is —CO2H.

The term “carboxyalkyl” is an alkyl radical substituted with a carboxy radical. More preferred are “lower carboxyalkyl” which are lower alkyl radicals as defined above, and may be additionally substituted on the alkyl radical with halo. Examples of such lower carboxyalkyl radicals include carboxymethyl, carboxyethyl and carboxypropyl.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “cyclooxygenase-2 selective inhibitor” is a compound able to inhibit cyclooxygenase-2 without significant inhibition of cyclooxygenase-1. Typically, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more typically, of at least 100. Even more typically, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “halo” is a halogen such as fluorine, chlorine, bromine or iodine.

The term “haloalkyl” is a radical wherein any one or more of the alkyl carbon atoms is substituted with halo as defined above. Specifically included are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” is a radical having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also includes radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “hydrido” is a single hydrogen atom (H). This hydrido radical may be attached, for example, to an oxygen atom to form a hydroxyl radical or two hydrido radicals may be attached to a carbon atom to form a methylene (—CH2-) radical.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “PPAR agonist” or “peroxisome proliferator activated receptor agonist” refers to a compound or composition, which when combined with PPAR, directly or indirectly stimulates or increases an in vivo or in vitro reaction typical for the receptor (e.g., transcriptional regulation activity). The increased reaction can be measured by any of a variety of assays known to those skilled in the art.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The term “subject” for purposes of treatment includes any human or animal subject who is need of treatment for an ischemic mediated central nervous system disorder or who is at risk for developing an ischemic mediated central nervous system disorder. The subject can be a domestic livestock species, a laboratory animal species, a zoo animal or a companion animal. In one embodiment, the subject is a mammal. In another embodiment, the mammal is a human being.

The term “sulfonyl”, whether used alone or linked to other terms such as alkylsulfonyl, is a divalent radical —SO₂—. “Alkylsulfonyl” is an alkyl radical attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrase “therapeutically-effective” is intended to qualify the amount of each agent (i.e. the amount of cyclooxygenase-2 selective inhibitor and the amount of peroxisome proliferator activated receptor agonist) which will achieve the goal of improvement in disorder severity and the frequency of incidence over no treatment or treatment of each agent by itself.

The term “thrombotic event” or “thromboembolic event” includes, but is not limited to arterial thrombosis, including stent and graft thrombosis, cardiac thrombosis, coronary thrombosis, heart valve thrombosis, pulmonary thrombosis and venous thrombosis. Cardiac thrombosis is thrombosis in the heart. Pulmonary thrombosis is thrombosis in the lung. Arterial thrombosis is thrombosis in an artery. Coronary thrombosis is the development of an obstructive thrombus in a coronary artery, often causing sudden death or a myocardial infarction. Venous thrombosis is thrombosis in a vein. Heart valve thrombosis is a thrombosis on a heart valve. Stent thrombosis is thrombosis resulting from and/or located in the vicinity of a vascular stent. Graft thrombosis is thrombosis resulting from and/or located in the vicinity of an implanted graft, particularly a vascular graft. A thrombotic event as used herein is meant to embrace both a local thrombotic event and a distal thrombotic event occurring anywhere within the body (e.g., a thromboembolic event such as for example an embolic stroke).

The term “vaso-occlusive event” includes a partial occlusion (including a narrowing) or complete occlusion of a blood vessel, a stent or a vascular graft. A vaso-occlusive event intends to embrace thrombotic or thromboembolic events, and the vascular occlusion disorders or conditions to which they give rise. Thus, a vaso-occlusive event is intended to embrace all vascular occlusive disorders resulting in partial or total vessel occlusion from thrombotic or thromboembolic events.

### DESCRIPTION OF THE PREFERRED EMBODIMENTS

The present invention provides a combination therapy comprising the administration to a subject of a therapeutically effective amount of a COX-2 selective inhibitor in combination with a therapeutically effective amount of a peroxisome proliferator activated receptor (PPAR) agonist. The combination therapy is used to treat or prevent ischemic mediated central nervous system damage, such as damage to a central nervous system cell resulting from a decrease in blood flow to the cell or damage resulting from a traumatic injury to the cell. In addition, the combination therapy may also be useful for the treatment of stroke or other vaso-occlusive events. When administered as part of a combination therapy, the COX-2 selective inhibitor together with the PPAR agonist provide enhanced treatment options as compared to administration of either the PPAR agonist or the COX-2 selective inhibitor alone.

Cyclooxygenase-2 Selective Inhibitors

A number of suitable cyclooxygenase-2 selective inhibitors or an isomer, a pharmaceutically acceptable salt, ester, or prodrug thereof, may be employed in the composition of the current invention. In one embodiment, the cyclooxygenase-2 selective inhibitor can be, for example, the cyclooxygenase-2 selective inhibitor meloxicam, Formula B-1 (CAS registry number 71125-38-7) or an isomer, a pharmaceutically acceptable salt, ester, or prodrug of a compound having Formula B-1.

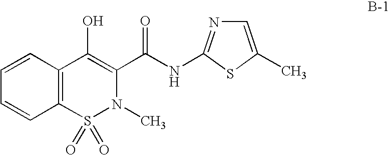

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or an isomer, a pharmaceutically acceptable salt, ester, or prodrug of a compound having Formula B-2.

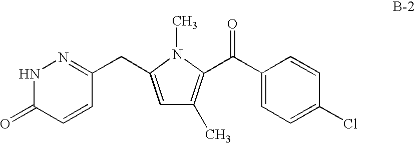

In still another embodiment the cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having Formula I shown below and possessing, by way of example and not limitation, the structures disclosed in Table 1. Furthermore, benzopyran cyclooxygenase-2 selective inhibitors useful in the practice of the present methods are described in U.S. Pat. Nos. 6,034,256 and 6,077,850 herein incorporated by reference in their entirety.

In another embodiment, the cyclooxygenase-2 selective inhibitor is a chromene compound represented by Formula I or an isomer, a pharmaceutically acceptable salt, ester, or prodrug thereof: 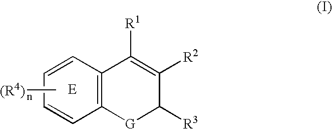
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof, wherein:
n is an integer which is 0, 1, 2, 3 or 4;
G is O, S or NR^{a};
R¹ is H;
R^{a} is alkyl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the cyclooxygenase-2 selective inhibitor may also be a compound of Formula (I), or an isomer, a pharmaceutically acceptable salt, ester, or prodrug thereof, wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:
R² is carboxyl;
R³ is lower haloalkyl; and
each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof wherein:
n is an integer which is 0, 1, 2, 3 or 4;
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:
n is an integer which is 0, 1, 2, 3 or 4;
R³ is trifluoromethyl or pentafluoroethyl; and
each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or an isomer, a pharmaceutically acceptable salt, ester, or prodrug thereof wherein:
n=4;
G is O or S;
R¹ is H;
R² is CO₂H;
R³ is lower haloalkyl;
a first R⁴ corresponding to R⁹ is hydrido or halo;
a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;
a third R⁴ corresponding to R¹¹ is H, lower alkyl, halo, lower alkoxy, or aryl; and
a fourth R⁴ corresponding to R¹² is H, halo, lower alkyl, lower alkoxy, or aryl;
wherein Formula (I) is represented by Formula (Ia):

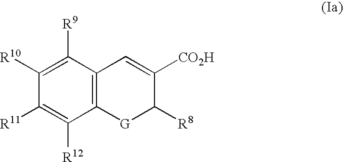

The cyclooxygenase-2 selective inhibitor used in connection with the method(s) of the present invention can also be a compound of having the structure of Formula (Ia) or an isomer, a pharmaceutically acceptable salt, ester, or prodrug thereof wherein:
R⁸ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl.

Examples of exemplary chromene cyclooxygenase-2 selective inhibitors are depicted in Table 1 below.

TABLE 1
EXAMPLES OF CHROMENE CYCLOOXYGENASE-2 SELECTIVE
INHIBITORS AS EMBODIMENTS

| Compound | |
|---|---|
| Number | Structural Formula |
| B-3 | 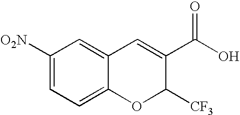 |
| | 6-Nitro-2-trifluoromethyl-2H-1- |
| | benzopyran-3-carboxylic acid |
| B-4 | 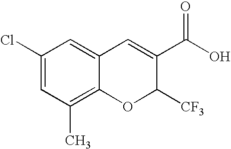 |
| | 6-Chloro-8-methyl-2-trifluoromethyl- |
| | 2H-1-benzopyran-3-carboxylic acid |
| B-5 | 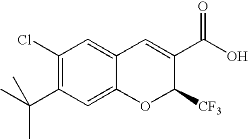 |
| | ((S)-6-Chloro-7-(1,1-dimethylethyl)-2- |
| | (trifluoromethyl-2H-1-benzopyran-3-carboxylic acid |
| B-6 | 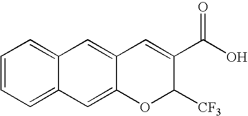 |
| | 2-Trifluoromethyl-2H-naphtho[2,3-b] |
| | pyran-3-carboxylic acid |
| B-7 | 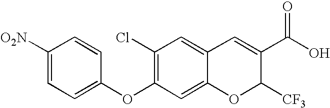 |
| | 6-Chloro-7-(4-nitrophenoxy)-2-(trifluoromethyl)- |
| | 2H-1-benzopyran-3-carboxylic acid |
| B-8 | 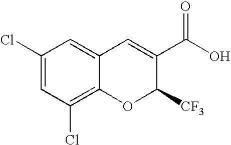 |
| | ((S)-6,8-Dichloro-2-(trifluoromethyl)- |
| | 2H-1-benzopyran-3-carboxyiic acid |
| B-9 | 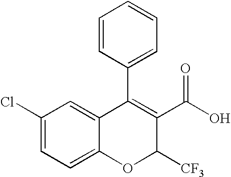 |
| | 6-Chloro-2-(trifluoromethyl)-4-phenyl-2H- |
| | 1-benzopyran-3-carboxylic acid |
| B-10 | 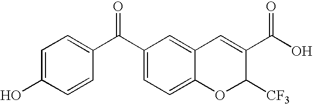 |
| | 6-(4-Hydroxybenzoyl)-2-(trifluoromethyl)- |
| | 2H-1-benzopyran-3-carboxyiic acid |
| B-11 | 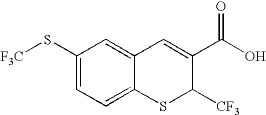 |
| | 2-(Trifluoromethyl)-6-[(trifluoromethyl)thio]- |
| | 2H-1-benzothiopyran-3-carboxyiic acid |
| B-12 | 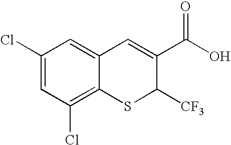 |
| | 6,8-Dichloro-2-trifluoromethyl-2H-1- |
| | benzothiopyran-3-carboxylic acid |
| B-13 | 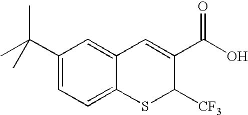 |
| | 6-(1,1-Dimethylethyl)-2-(trifluoromethyl)- |
| | 2H-1-benzothiopyran-3-carboxylic acid |
| B-14 | 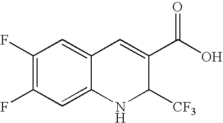 |
| | 6,7-Difluoro-1,2-dihydro-2-(trifluoro- |
| | methyl)-3-quinolinecarboxylic acid |
| B-15 | 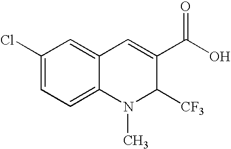 |
| | 6-Chloro-1,2-dihydro-1-methyl-2-(trifluoro- |
| | methyl)-3-quinolinecarboxylic acid |
| B-16 | 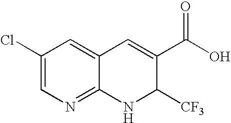 |
| | 6-Chloro-2-(trifluoromethyl)-1,2-dihydro |
| | [1,8]naphthyridine-3-carboxylic acid |
| B-17 | 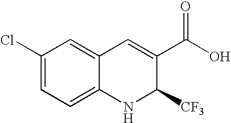 |
| | ((S)-6-Chloro-1,2-dihydro-2-(trifluoro- |
| | methyl)-3-quinolinecarboxylic acid |

In a further embodiment, the cyclooxygenase-2 selective inhibitor is selected from the class of tricyclic cyclooxygenase-2 selective inhibitors represented by the general structure of Formula I: or an isomer, a pharmaceutically acceptable salt, ester, or prodrug thereof wherein:

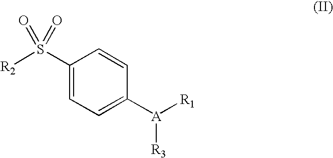

A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

In another embodiment, the cyclooxygenase-2 selective inhibitor represented by the above Formula II is selected from the group of compounds illustrated in Table 2, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), and tilmacoxib (JTE-522; B-23; CAS No. 180200-68-4).

TABLE 2
EXAMPLES OF TRICYCLIC CYCLOOXYGENASE-2 SELECTIVE
INHIBITORS AS EMBODIMENTS

| Compound | |
|---|---|
| Number | Structural Formula |
| B-18 | 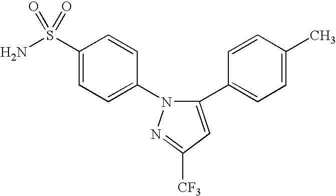 |
| B-19 | 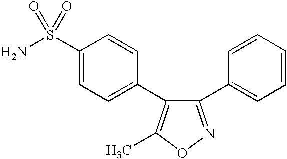 |
| B-20 | 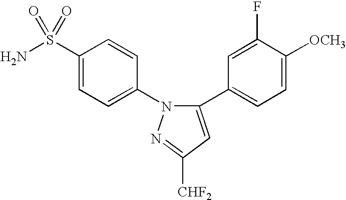 |
| B-21 | 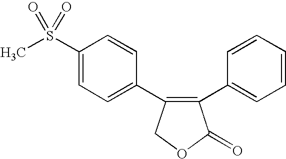 |
| B-22 | 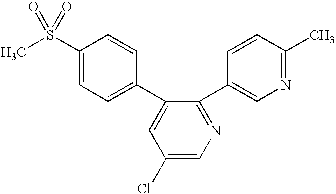 |
| B-23 | 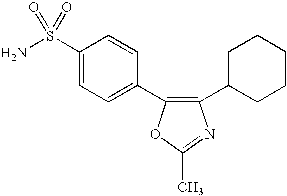 |

In still another embodiment, the cyclooxygenase-2 selective inhibitor is selected from the group consisting of celecoxib, rofecoxib and etoricoxib.

In yet another embodiment, the cyclooxygenase-2 selective inhibitor is parecoxib (B-24, U.S. Pat. No. 5,932,598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

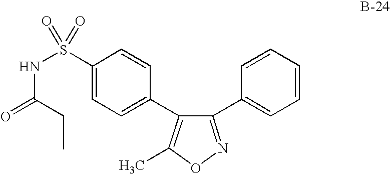

One form of parecoxib is sodium parecoxib.

In another embodiment of the invention, the compound having the formula B-25 or an isomer, a pharmaceutically acceptable salt, ester, or prodrug of a compound having formula B-25 that has been previously described in International Publication number WO 00/24719 (which is herein incorporated by reference) is another tricyclic cyclooxygenase-2 selective inhibitor that may be advantageously employed.

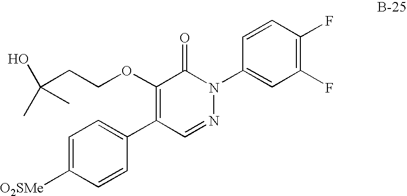

Another cyclooxygenase-2 selective inhibitor that is useful in connection with the method(s) of the present invention is N-(2-cyclohexyloxynitrophenyl)-methane sulfonamide (NS-398) having a structure shown below as B-26, or an isomer, a pharmaceutically acceptable salt, ester, or prodrug of a compound having formula B-26.

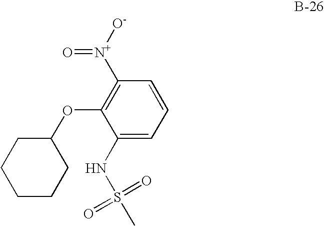

In yet a further embodiment, the cyclooxygenase-2 selective inhibitor used in connection with the method(s) of the present invention can be selected from the class of phenylacetic acid derivative cyclooxygenase-2 selective inhibitors represented by the general structure of Formula (III) or an isomer, a pharmaceutically acceptable salt, ester, or prodrug thereof: 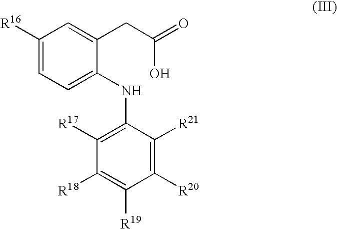
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl, provided, however, that each of R¹⁷, R¹⁸, R¹⁹ and R²⁰ is not fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (lumiracoxib; B-211) and that has the structure shown in Formula (III) or an isomer, a pharmaceutically acceptable salt, ester, or prodrug thereof wherein:
R¹⁶ is ethyl;
R¹⁷ and R¹⁹ are chloro;
R¹⁸ and R²⁰ are hydrogen; and
R²¹ is methyl.

In yet another embodiment, the cyclooxygenase-2 selective inhibitor is represented by Formula (IV) or an isomer, a pharmaceutically acceptable salt, ester, or prodrug thereof: 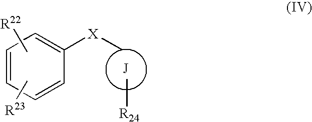
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

According to another embodiment, the cyclooxygenase-2 selective inhibitors used in the present method(s) have the structural Formula (V) or an isomer, a pharmaceutically acceptable salt, ester, or prodrug thereof: 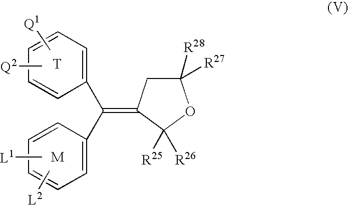
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

In another embodiment, the compounds N-(2-cyclohexyloxynitrophenyl)methane sulfonamide, and (E)-4-[(4-methylphenyl)(tetrahydro-2-oxo-3-furanylidene)methyl]benzenesulfonamide or an isomer, a pharmaceutically acceptable salt, ester, or prodrug thereof having the structure of Formula (V) are employed as cyclooxygenase-2 selective inhibitors.

In a further embodiment, compounds that are useful for the cyclooxygenase-2 selective inhibitor or an isomer, a pharmaceutically acceptable salt, ester, or prodrug thereof used in connection with the method(s) of the present invention, the structures for which are set forth in Table 3 below, include, but are not limited to:
6-chloro-2-trifluoromethyl-2H-1-benzopyran-3-carboxylic acid (B-27);
6-chloro-7-methyl-2-trifluoromethyl-2H-1-benzopyran-3-carboxylic acid (B-28);
8-(1-methylethyl)-2-trifluoromethyl-2H-1-benzopyran-3-carboxylic acid (B-29);
6-chloro-8-(1-methylethyl)-2-trifluoromethyl-2H-1-benzopyran-3-carboxylic acid (B-30);
2-trifluoromethyl-3H-naphtho[2,1-b]pyran-3-carboxylic acid (B-31);
7-(1,1-dimethylethyl)-2-trifluoromethyl-2H-1-benzopyran-3-carboxylic acid (B-32);
6-bromo-2-trifluoromethyl-2H-1-benzopyran-3-carboxylic acid (B-33);
8-chloro-2-trifluoromethyl-2H-1-benzopyran-3-carboxylic acid (B-34);
6-trifluoromethoxy-2-trifluoromethyl-2H-1-benzopyran-3-carboxylic acid (B-35);
5,7-dichloro-2-trifluoromethyl-2H-1-benzopyran-3-carboxylic acid (B-36);
8-phenyl-2-trifluoromethyl-2H-1-benzopyran-3-carboxylic acid (B-37);
7,8-dimethyl-2-trifluoromethyl-2H-1-benzopyran-3-carboxylic acid (B-38);
6,8-bis(dimethylethyl)-2-trifluoromethyl-2H-1-benzopyran-3-carboxylic acid (B-39);
7-(1-methylethyl)-2-trifluoromethyl-2H-1-benzopyran-3-carboxylic acid (B-40);
7-phenyl-2-trifluoromethyl-2H-1-benzopyran-3-carboxylic acid (B-41);
6-chloro-7-ethyl-2-trifluoromethyl-2H-1-benzopyran-3-carboxylic acid (B-42);
6-chloro-8-ethyl-2-trifluoromethyl-2H-1-benzopyran-3-carboxylic acid (B-43);
6-chloro-7-phenyl-2-trifluoromethyl-2H-1-benzopyran-3-carboxylic acid (B-44);
6,7-dichloro-2-trifluoromethyl-2H-1-benzopyran-3-carboxylic acid (B-45);
6,8-dichloro-2-trifluoromethyl-2H-1-benzopyran-3-carboxylic acid (B-46);
6-chloro-8-methyl-2-trifluoromethyl-2H-1-benzopyran-3-carboxylic acid (B-47);
8-chloro-6-methyl-2-trifluoromethyl-2H-1-benzopyran-3-carboxylic acid (B-48)
8-chloro-6-methoxy-2-trifluoromethyl-2H-1-benzopyran-3-carboxylic acid (B-49);
6-bromo-8-chloro-2-trifluoromethyl-2H-1-benzopyran-3-carboxylic acid (B-50);
8-bromo-6-fluoro-2-trifluoromethyl-2H-1-benzopyran-3-carboxylic acid (B-51);
8-bromo-6-methyl-2-trifluoromethyl-2H-1-benzopyran-3-carboxylic acid (B-52);
8-bromo-5-fluoro-2-trifluoromethyl-2H-1-benzopyran-3-carboxylic acid (B-53);
6-chloro-8-fluoro-2-trifluoromethyl-2H-1-benzopyran-3-carboxylic acid (B-54);
6-bromo-8-methoxy-2-trifluoromethyl-2H-1-benzopyran-3-carboxylic acid (B-55);
6-[[(phenylmethyl)amino]sulfonyl]-2-trifluoromethyl-2H-1-benzopyran-3-carboxylic acid (B-56);
6-[(dimethylamino)sulfonyl]-2-trifluoromethyl-2H-1-benzopyran-3-carboxylic acid (B-57);
6-[(methylamino)sulfonyl]-2-trifluoromethyl-2H-1-benzopyran-3-carboxylic acid (B-58);
6-[(4-morpholino)sulfonyl]-2-trifluoromethyl-2H-1-benzopyran-3-carboxylic acid (B-59);
6-[(1,1-dimethylethyl)aminosulfonyl]-2-trifluoromethyl-2H-1-benzopyran-3-carboxylic acid (B-60);
6-[(2-methylpropyl)aminosulfonyl]-2-trifluoromethyl-2H-1-benzopyran-3-carboxylic acid (B-61);
6-methylsulfonyl-2-trifluoromethyl-2H-1-benzopyran-3-carboxylic acid (B-62);
8-chloro-6-[[(phenylmethyl)amino]sulfonyl]-2-trifluoromethyl-2H-1-benzopyran-3-carboxylic acid (B-63);
6-phenylacetyl-2-trifluoromethyl-2H-1-benzopyran-3-carboxylic acid (B-64);
6,8-dibromo-2-trifluoromethyl-2H-1-benzopyran-3-carboxylic acid (B-65);
8-chloro-5,6-dimethyl-2-trifluoromethyl-2H-1-benzopyran-3-carboxylic acid (B-66);
6,8-dichloro-(S)-2-trifluoromethyl-2H-1-benzopyran-3-carboxylic acid (B-67);
6-benzylsulfonyl-2-trifluoromethyl-2H-1-benzopyran-3-carboxylic acid (B-68);
6-[[N-(2-furylmethyl)amino]sulfonyl]-2-trifluoromethyl-2H-1-benzopyran-3-carboxylic acid (B-69);
6-[[N-(2-phenylethyl)amino]sulfonyl]-2-trifluoromethyl-2H-1-benzopyran-3-carboxylic acid (B-70);
6-iodo-2-trifluoromethyl-2H-1-benzopyran-3-carboxylic acid (B-71);
7-(1,1-dimethylethyl)-2-pentafluoroethyl-2H-1-benzopyran-3-carboxylic acid (B-72);
6-chloro-2-trifluoromethyl-2H-1-benzothiopyran-3-carboxylic acid (B-73);
3-[(3-Chloro-phenyl)-(4-methanesulfonyl-phenyl)-methylene]-dihydro-furan-2-one or BMS-347070 (B-74);
8-acetyl-3-(4-fluorophenyl)-2-(4-methylsulfonyl)phenyl-imidazo(1,2-a)pyridine (B-75);
5,5-dimethyl-4-(4-methylsulfonyl)phenyl-3-phenyl-2-(5H)-furanone (B-76);
5-(4-fluorophenyl)-1-[4-(methylsulfonyl)phenyl]-3-(trifluoromethyl)pyrazole (B-77);
4-(4-fluorophenyl)-5-[4-(methylsulfonyl)phenyl]-1-phenyl-3-(trifluoromethyl)pyrazole (B-78);
4-(5-(4-chlorophenyl)-3-(4-methoxyphenyl)-1H-pyrazol-1-yl) benzenesulfonamide (B-79);
4-(3,5-bis(4-methylphenyl)-1H-pyrazol-1-yl)benzenesulfonamide (B-80);
4-(5-(4-chlorophenyl)-3-phenyl-1H-pyrazol-1-yl)benzenesulfonamide (B-81);
4-(3,5-bis(4-methoxyphenyl)-1H-pyrazol-1-yl)benzenesulfonamide (B-82);
4-(5-(4-chlorophenyl)-3-(4-methylphenyl)-1H-pyrazol-1-yl) benzenesulfonamide (B-83);
4-(5-(4-chlorophenyl)-3-(4-nitrophenyl)-1H-pyrazol-1-yl) benzenesulfonamide (B-84);
4-(5-(4-chlorophenyl)-3-(5-chloro-2-thienyl)-1H-pyrazol-1-yl) benzenesulfonamide (B-85);
4-(4-chloro-3,5-diphenyl-1H-pyrazol-1-yl)benzenesulfonamide (B-86);
4-[5-(4-chlorophenyl)-3-(trifluoromethyl)-1H-pyrazol-1-yl]benzenesulfonamide (B-87);
4-[5-phenyl-3-(trifluoromethyl)-1H-pyrazol-1-yl]benzenesulfonamide (B-88);
4-[5-(4-fluorophenyl)-3-(trifluoromethyl)-1H-pyrazol-1-yl]benzenesulfonamide (B-89);
4-[5-(4-methoxyphenyl)-3-(trifluoromethyl)-1H-pyrazol-1-yl]benzenesulfonamide (B-90);
4-[5-(4-chlorophenyl)-3-(difluoromethyl)-1H-pyrazol-1-yl]benzenesulfonamide (B-91);
4-[5-(4-methylphenyl)-3-(trifluoromethyl)-1H-pyrazol-1-yl]benzenesulfonamide (B-92);
4-[4-chloro-5-(4-chlorophenyl)-3-(trifluoromethyl)-1H-pyrazol-1-yl]benzenesulfonamide (B-93);
4-[3-(difluoromethyl)-5-(4-methylphenyl)-1H-pyrazol-1-yl]benzenesulfonamide (B-94);
4-[3-(difluoromethyl)-5-phenyl-1H-pyrazol-1-yl]benzenesulfonamide (B-95);
4-[3-(difluoromethyl)-5-(4-methoxyphenyl)-1H-pyrazol-1-yl]benzenesulfonamide (B-96);
4-[3-cyano-5-(4-fluorophenyl)-1H-pyrazol-1-yl]benzenesulfonamide (B-97);
4-[3-(difluoromethyl)-5-(3-fluoro-4-methoxyphenyl)-1H-pyrazol-1-yl]benzenesulfonamide (B-98);
4-[5-(3-fluoro-4-methoxyphenyl)-3-(trifluoromethyl)-1H-pyrazol-1-yl]benzenesulfonamide (B-99);
4-[4-chloro-5-phenyl-1H-pyrazol-1-yl]benzenesulfonamide (B-100);
4-[5-(4-chlorophenyl)-3-(hydroxymethyl)-1H-pyrazol-1-yl]benzenesulfonamide (B-101);
4-[5-(4-(N,N-dimethylamino)phenyl)-3-(trifluoromethyl)-1H-pyrazol-1-yl]benzenesulfonamide (B-102);
5-(4-fluorophenyl)-6-[4-(methylsulfonyl)phenyl]spiro[2.4]hept-5-ene (B-103);
4-[6-(4-fluorophenyl)spiro[2.4]hept-5-en-5-yl]benzenesulfonamide (B-104);
6-(4-fluorophenyl)-7-[4-(methylsulfonyl)phenyl]spiro[3.4]oct-6-ene (B-105);
5-(3-chloro-4-methoxyphenyl)-6-[4-(methylsulfonyl)phenyl]spiro[2.4]hept-5-ene (B-106);
4-[6-(3-chloro-4-methoxyphenyl)spiro[2.4]hept-5-en-5-yl]benzenesulfonamide (B-107);
5-(3,5-dichloro-4-methoxyphenyl)-6-[4-(methylsulfonyl)phenyl]spiro[2.4]hept-5-ene (B-108);
5-(3-chloro-4-fluorophenyl)-6-[4-(methylsulfonyl)phenyl]spiro[2.4]hept-5-ene (B-109);
4-[6-(3,4-dichlorophenyl)spiro[2.4]hept-5-en-5-yl]benzenesulfonamide (B-110);
2-(3-chloro-4-fluorophenyl)-4-(4-fluorophenyl)-5-(4-methylsulfonylphenyl)thiazole (B-111);
2-(2-chlorophenyl)-4-(4-fluorophenyl)-5-(4-methylsulfonylphenyl)thiazole (B-112);
5-(4-fluorophenyl)-4-(4-methylsulfonylphenyl)-2-methylthiazole (B-113);
4-(4-fluorophenyl)-5-(4-methylsulfonylphenyl)-2-trifluoromethylthiazole (B-114);
4-(4-fluorophenyl)-5-(4-methylsulfonylphenyl)-2-(2-thienyl)thiazole (B-115);
4-(4-fluorophenyl)-5-(4-methylsulfonylphenyl)-2-benzylaminothiazole (B-116);
4-(4-fluorophenyl)-5-(4-methylsulfonylphenyl)-2-(1-propylamino)thiazole (B-117);
2-[(3,5-dichlorophenoxy)methyl)-4-(4-fluorophenyl)-5-[4-(methylsulfonyl)phenyl]thiazole (B-118);
5-(4-fluorophenyl)-4-(4-methylsulfonylphenyl)-2-trifluoromethylthiazole (B-119);
1-methylsulfonyl-4-[1,1-dimethyl-4-(4-fluorophenyl)cyclopenta-2,4-dien-3-yl]benzene (B-120);
4-[4-(4-fluorophenyl)-1,1-dimethylcyclopenta-2,4-dien-3-yl]benzenesulfonamide (B-121);
5-(4-fluorophenyl)-6-[4-(methylsulfonyl)phenyl]spiro[2.4]hepta-4,6-diene (B-122);
4-[6-(4-fluorophenyl)spiro[2.4]hepta-4,6-dien-5-yl]benzenesulfonamide (B-123);
6-(4-fluorophenyl)-2-methoxy-5-[4-(methylsulfonyl)phenyl]-pyridine-3-carbonitrile (B-124);
2-bromo-6-(4-fluorophenyl)-5-[4-(methylsulfonyl)phenyl]-pyridine-3-carbonitrile (B-125);
6-(4-fluorophenyl)-5-[4-(methylsulfonyl)phenyl]-2-phenyl-pyridine-3-carbonitrile (B-126);
4-[2-(4-methylpyridin-2-yl)-4-(trifluoromethyl)-1H-imidazol-1-yl]benzenesulfonamide (B-127);
4-[2-(5-methylpyridin-3-yl)-4-(trifluoromethyl)-1H-imidazol-1-yl]benzenesulfonamide (B-128);
4-[2-(2-methylpyridin-3-yl)-4-(trifluoromethyl)-1H-imidazol-1-yl]benzenesulfonamide (B-129);
3-[1-[4-(methylsulfonyl)phenyl]4-(trifluoromethyl)-1H-imidazol-2-yl]pyridine (B-130);
2-[1-[4-(methylsulfonyl)phenyl-4-(trifluoromethyl)-1H-imidazol-2-yl]pyridine (B-131);
2-methyl-4-[1-[4-(methylsulfonyl)phenyl-4-(trifluoromethyl)-1H-imidazol-2-yl]pyridine (B-132);
2-methyl-6-[1-[4-(methylsulfonyl)phenyl-4-(trifluoromethyl)-1H-imidazol-2-yl]pyridine (B-133);
4-[2-(6-methylpyridin-3-yl)-4-(trifluoromethyl)-1H-imidazol-1-yl]benzenesulfonamide (B-134);
2-(3,4-difluorophenyl)-1-[4-(methylsulfonyl)phenyl]4-(trifluoromethyl)-1H-imidazole (B-135);
4-[2-(4-methylphenyl)-4-(trifluoromethyl)-1H-imidazol-1-yl]benzenesulfonamide (B-136);
2-(4-chlorophenyl)-1-[4-(methylsulfonyl)phenyl]4-methyl-1H-imidazole (B-137);
2-(4-chlorophenyl)-1-[4-(methylsulfonyl)phenyl]4-phenyl-1H-imidazole (B-138);
2-(4-chlorophenyl)-4-(4-fluorophenyl)-1-[4-(methylsulfonyl)phenyl]-1H-imidazole (B-139);
2-(3-fluoro-4-methoxyphenyl)-1-[4-(methylsulfonyl)phenyl-4-(trifluoromethyl)-1H-imidazole (B-140);
1-[4-(methylsulfonyl)phenyl]-2-phenyl-4-trifluoromethyl-1H-imidazole (B-141);
2-(4-methylphenyl)-1-[4-(methylsulfonyl)phenyl]-4-trifluoromethyl-1H-imidazole (B-142);
4-[2-(3-chloro-4-methylphenyl)-4-(trifluoromethyl)-1H-imidazol-1-yl]benzenesulfonamide (B-143);
2-(3-fluoro-5-methylphenyl)-1-[4-(methylsulfonyl)phenyl]-4-(trifluoromethyl)-1H-imidazole (B-144);
4-[2-(3-fluoro-5-methylphenyl)-4-(trifluoromethyl)-1H-imidazol-1-yl]benzenesulfonamide (B-145);
2-(3-methylphenyl)-1-[4-(methylsulfonyl)phenyl]-4-trifluoromethyl-1H-imidazole (B-146);
4-[2-(3-methylphenyl)-4-trifluoromethyl-1H-imidazol-1-yl]benzenesulfonamide (B-147);
1-[4-(methylsulfonyl)phenyl]-2-(3-chlorophenyl)-4-trifluoromethyl-1H-imidazole (B-148);
4-[2-(3-chlorophenyl)-4-trifluoromethyl-1H-imidazol-1-yl]benzenesulfonamide (B-149);
4-[2-phenyl-4-trifluoromethyl-1H-imidazol-1-yl]benzenesulfonamide (B-150);
4-[2-(4-methoxy-3-chlorophenyl)-4-trifluoromethyl-1H-imidazol-1-yl]benzenesulfonamide (B-151);
1-allyl-4-(4-fluorophenyl)-3-[4-(methylsulfonyl)phenyl]-5-(trifluoromethyl)-1H-pyrazole (B-152);
4-[1-ethyl-4-(4-fluorophenyl)-5-(trifluoromethyl)-1H-pyrazol-3-yl]benzenesulfonamide (B-153);
N-phenyl-[4-(4-fluorophenyl)-3-[4-(methylsulfonyl)phenyl]-5-(trifluoromethyl)-1H-pyrazol-1-yl]acetamide (B-154);
ethyl [4-(4-fluorophenyl)-3-[4-(methylsulfonyl)phenyl]-5-(trifluoromethyl)-1H-pyrazol-1-yl]acetate (B-155);
4-(4-fluorophenyl)-3-[4-(methylsulfonyl)phenyl]-1-(2-phenylethyl)-1H-pyrazole (B-156);
4-(4-fluorophenyl)-3-[4-(methylsulfonyl)phenyl]-1-(2-phenylethyl)-5-(trifluoromethyl)pyrazole (B-157);
1-ethyl-4-(4-fluorophenyl)-3-[4-(methylsulfonyl)phenyl]-5-(trifluoromethyl)-1H-pyrazole (B-158);
5-(4-fluorophenyl)-4-(4-methylsulfonylphenyl)-2-trifluoromethyl-1H-imidazole (B-159);
4-[4-(methylsulfonyl)phenyl]-5-(2-thiophenyl)-2-(trifluoromethyl)-1H-imidazole (B-160);
5-(4-fluorophenyl)-2-methoxy-4-[4-(methylsulfonyl)phenyl]-6-(trifluoromethyl)pyridine (B-161);
2-ethoxy-5-(4-fluorophenyl)-4-[4-(methylsulfonyl)phenyl]-6-(trifluoromethyl)pyridine (B-162);
5-(4-fluorophenyl)-4-[4-(methylsulfonyl)phenyl]-2-(2-propynyloxy)-6-(trifluoromethyl)pyridine (B-163);
2-bromo-5-(4-fluorophenyl)-4-[4-(methylsulfonyl)phenyl]-6-(trifluoromethyl)pyridine (B-164);
4-[2-(3-chloro-4-methoxyphenyl)-4,5-difluorophenyl]benzenesulfonamide (B-165);
1-(4-fluorophenyl)-2-[4-(methylsulfonyl)phenyl]benzene (B-166);
5-difluoromethyl-4-(4-methylsulfonylphenyl)-3-phenylisoxazole (B-167);
4-[3-ethyl-5-phenylisoxazol-4-yl]benzenesulfonamide (B-168);
4-[5-difluoromethyl-3-phenylisoxazol-4-yl]benzenesulfonamide (B-169);
4-[5-hydroxymethyl-3-phenylisoxazol-4-yl]benzenesulfonamide (B-170);
4-[5-methyl-3-phenyl-isoxazol-4-yl]benzenesulfonamide (B-171);
1-[2-(4-fluorophenyl)cyclopenten-1-yl]4-(methylsulfonyl)benzene (B-172);
1-[2-(4-fluoro-2-methylphenyl)cyclopenten-1-yl]4-(methylsulfonyl)benzene (B-173);
1-[2-(4-chlorophenyl)cyclopenten-1-yl]4-(methylsulfonyl)benzene (B-174);
1-[2-(2,4-dichlorophenyl)cyclopenten-1-yl]4-(methylsulfonyl)benzene (B-175);
1-[2-(4-trifluoromethylphenyl)cyclopenten-1-yl]4-(methylsulfonyl)benzene (B-176);
1-[2-(4-methylthiophenyl)cyclopenten-1-yl]-4-(methylsulfonyl)benzene (B-177);
1-[2-(4-fluorophenyl)-4,4-dimethylcyclopenten-1-yl]4-(methylsulfonyl)benzene (B-178);
4-[2-(4-fluorophenyl)-4,4-dimethylcyclopenten-1-yl]benzenesulfonamide (B-179);
1-[2-(4-chlorophenyl)-4,4-dimethylcyclopenten-1-yl]-4-(methylsulfonyl)benzene (B-180);
4-[2-(4-chlorophenyl)-4,4-dimethylcyclopenten-1-yl]benzenesulfonamide (B-181);
4-[2-(4-fluorophenyl)cyclopenten-1-yl]benzenesulfonamide (B-182);
4-[2-(4-chlorophenyl)cyclopenten-1-yl]benzenesulfonamide (B-183);
1-[2-(4-methoxyphenyl)cyclopenten-1-yl]4-(methylsulfonyl)benzene (B-184);
1-[2-(2,3-difluorophenyl)cyclopenten-1-yl]4-(methylsulfonyl)benzene (B-185);
4-[2-(3-fluoro-4-methoxyphenyl)cyclopenten-1-yl]benzenesulfonamide (B-186);
1-[2-(3-chloro-4-methoxyphenyl)cyclopenten-1-yl]4-(methylsulfonyl)benzene (B-187);
4-[2-(3-chloro-4-fluorophenyl)cyclopenten-1-yl]benzenesulfonamide (B-188);
4-[2-(2-methylpyridin-5-yl)cyclopenten-1-yl]benzenesulfonamide (B-189);
ethyl 2-[4-(4-fluorophenyl)-5-[4-(methylsulfonyl)phenyl]oxazol-2-yl]-2-benzyl-acetate (B-190);
2-[4-(4-fluorophenyl)-5-[4-(methylsulfonyl)phenyl]oxazol-2-yl]acetic acid (B-191);
2-(tert-butyl)-4-(4-fluorophenyl)-5-[4-(methylsulfonyl)phenyl]oxazole (B-192);
4-(4-fluorophenyl)-5-[4-(methylsulfonyl)phenyl]-2-phenyloxazole (B-193);
4-(4-fluorophenyl)-2-methyl-5-[4-(methylsulfonyl)phenyl]oxazole (B-194);
4-[5-(3-fluoro-4-methoxyphenyl)-2-trifluoromethyl-4-oxazolyl]benzenesulfonamide (B-195);
6-chloro-7-(1,1-dimethylethyl)-2-trifluoromethyl-2H-1-benzopyran-3-carboxylic acid (B-196);
6-chloro-8-methyl-2-trifluoromethyl-2H-1-benzopyran-3-carboxylic acid (B-197);
5,5-dimethyl-3-(3-fluorophenyl)-4-methylsulfonyl-2(5H)-furanone (B-198);
6-chloro-2-trifluoromethyl-2H-1-benzothiopyran-3-carboxylic acid (B-199);
4-[5-(4-chlorophenyl)-3-(trifluoromethyl)-1H-pyrazol-1-yl]benzenesulfonamide (B-200);
4-[5-(4-methylphenyl)-3-(trifluoromethyl)-1H-pyrazol-1-yl]benzenesulfonamide (B-201);
4-[5-(3-fluoro-4-methoxyphenyl)-3-(difluoromethyl)-1H-pyrazol-1-yl]benzenesulfonamide (B-202);
3-[1-[4-(methylsulfonyl)phenyl]4-trifluoromethyl-1H-imidazol-2-yl]pyridine (B-203);
2-methyl-5-[1-[4-(methylsulfonyl)phenyl]4-trifluoromethyl-1H-imidazol-2-yl]pyridine (B-204);
4-[2-(5-methylpyridin-3-yl)-4-(trifluoromethyl)-1H-imidazol-1-yl]benzenesulfonamide (B-205);
4-[5-methyl-3-phenylisoxazol-4-yl]benzenesulfonamide (B-206);
4-[5-hydroxymethyl-3-phenylisoxazol-4-yl]benzenesulfonamide (B-207);
[2-trifluoromethyl-5-(3,4-difluorophenyl)-4-oxazolyl]benzenesulfonamide (B-208);
4-[2-methyl-4-phenyl-5-oxazolyl]benzenesulfonamide (B-209);
4-[5-(2-fluoro-4-methoxyphenyl)-2-trifluoromethyl-4-oxazolyl]benzenesulfonamide (B-210);
[2-(2-chloro-6-fluoro-phenylamino)-5-methyl-phenyl]-acetic acid or COX 189 (lumiracoxib; B-211);
N-(4-Nitro-2-phenoxy-phenyl)-methanesulfonamide or nimesulide (B-212);
N-[6-(2,4-difluoro-phenoxy)-1-oxo-indan-5-yl]-methanesulfonamide or flosulide (B-213);
N-[6-(2,4-Difluoro-phenylsulfanyl)-1-oxo-1H-inden-5-yl]-methanesulfonamide, sodium salt or L-745337 (B-214);
N-[5-(4-fluoro-phenylsulfanyl)-thiophen-2-yl]-methanesulfonamide or RWJ-63556 (B-215);
3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);
(5Z)-2-amino-5-[[3,5-bis(1,1-dimethylethyl)-4-hydroxyphenyl]methylene]-4(5H)-thiazolone or darbufelone (B-217);
CS-502 (B-218);
LAS-34475 (B-219);
LAS-34555 (B-220);
S-33516 (B-221);
SD-8381 (B-222);
L-783003 (B-223);
N-[3-(formylamino)-4-oxo-6-phenoxy-4H-1-benzopyran-7-yl]-methanesulfonamide or T-614 (B-224);
D-1367 (B-225);
L-748731 (B-226);
(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);
CGP-28238 (B-228);
4-[[3,5-bis(1,1-dimethylethyl)-4-hydroxyphenyl]methylene]dihydro-2-methyl-2H-1,2-oxazin-3(4H)-one or BF-389 (B-229);
GR-253035 (B-230);
6-dioxo-9H-purin-8-yl-cinnamic acid (B-231);
S-2474 (B-232);
4-[4-(methyl)-sulfonyl)phenyl]-3-phenyl-2(5H)-furanone;
4-(5-methyl-3-phenyl-4-isoxazolyl);
2-(6-methylpyrid-3-yl)-3-(4-methylsulfonylphenyl)-5-chloropyridine;
4-[5-(4-methylphenyl)-3-(trifluoromethyl)-1H-pyrazol-1-yl];
N-[[4-(5-methyl-3-phenyl-4-isoxazolyl)phenyl]sulfonyl];
4-[5-(3-fluoro-4-methoxyphenyl)-3-difluoromethyl)-1H-pyrazol-1-yl]benzenesulfonamide;
(S)-6,8-dichloro-2-(trifluoromethyl)-2H-1-benzopyran-3-carboxylic acid;
2-(3,4-difluorophenyl)-4-(3-hydroxy-3-methylbutoxy)-5-[4-(methylsulfonyl)phenyl]-3(2H)-pyridzainone;
2-trifluoromethyl-3H-naptho[2,1-b]pyran-3-carboxylic acid;
6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3
EXAMPLES OF CYCLOOXYGENASE-2 SELECTIVE
INHIBITORS AS EMBODIMENTS

| Compound | |
|---|---|
| Number | Structural Formula |
| B-26 | 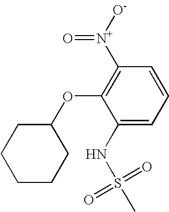 |
| | N-(2-cyclohexyloxynitrophenyl) methane sulfonamide or |
| | NS-398; |
| B-27 | 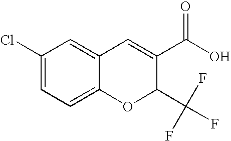 |
| | 6-chloro-2-trifluoromethyl-2H-1-benzopyran-3- |
| | carboxylic acid; |
| B-28 | 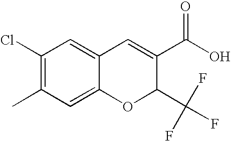 |
| | 6-chloro-7-methyl-2-trifluoromethyl-2H-1- |
| | benzopyran-3-carboxylic acid; |
| B-29 | 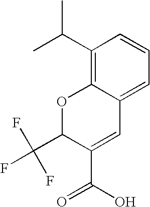 |
| | 8-(1-methylethyl)-2-trifluoromethyl-2H-1- |
| | benzopyran-3-carboxylic acid; |
| B-30 | 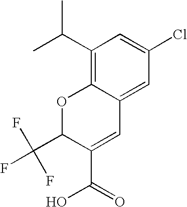 |
| | 6-chloro-8-(1-methylethyl)-2-trifluoromethyl- |
| | 2H-1-benzopyran-3-carboxylic acid; |
| B-31 | 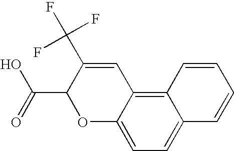 |
| | 2-trifluoromethyl-3H-naphtho[2,1-b]pyran-3- |
| | carboxylic acid; |
| B-32 | 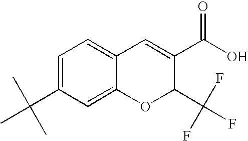 |
| | 7-(1,1-dimethylethyl)-2-trifluoromethyl-2H- |
| | 1-benzopyran-3-carboxylic acid; |
| B-33 | 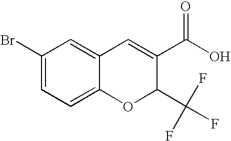 |
| | 6-bromo-2-trifluoromethyl-2H-1-benzopyran-3- |
| | carboxylic acid; |
| B-34 | 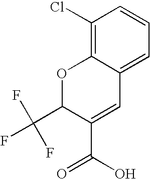 |
| | 8-chloro-2-trifluoromethyl-2H-1-benzopyran-3- |
| | carboxylic acid; |
| B-35 | 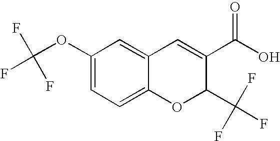 |
| | 6-trifluoromethoxy-2-trffluoromethyl-2H-1- |
| | benzopyran-3-carboxylic acid; |
| B-36 | 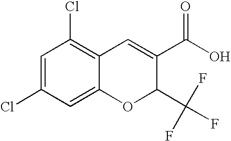 |
| | 5,7-dicbloro-2-tnfluoromethyl-2H-1-benzopyran- |
| | 3-carboxylic acid; |
| B-37 | 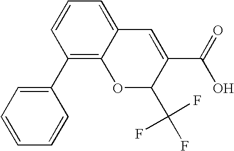 |
| | 8-phenyl-2-trifluoromethyl-2H-1-benzopyran- |
| | 3-carboxylic acid; |
| B-38 | 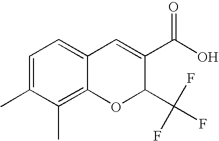 |
| | 7,8-dimethyl-2-trifluoromethyl-2H-1-benzopyran- |
| | 3-carboxylic acid; |
| B-39 | 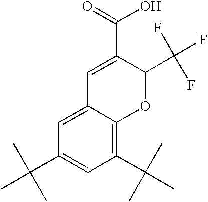 |
| | 6,8-bis(dimethylethyl)-2-trifluoromethyl-2H-1- |
| | benzopyran-3-carboxylic acid; |
| B-40 | 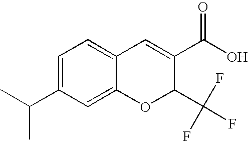 |
| | 7-(1-methylethyl)-2-trifluoromethyl-2H-1- |
| | benzopyran-3-carboxylic acid; |
| B-41 | 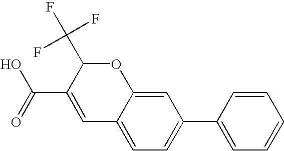 |
| | 7-phenyl-2-trifluoromethyl-2H-1-benzopyran-3- |
| | carboxylic acid; |
| B-42 | 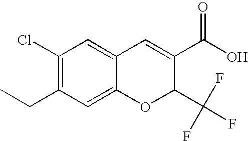 |
| | 6-chloro-7-ethyl-2-trifluoromethyl-2H-1- |
| | benzopyran-3-carboxylic acid; |
| B-43 | 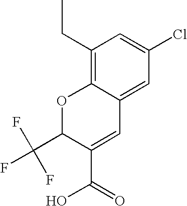 |
| | 6-chloro-8-ethyl-2-trifluoromethyl-2H-1- |
| | benzopyran-3-carboxylic acid; |
| B-44 | 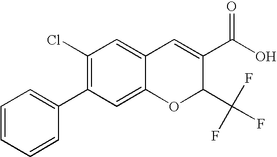 |
| | 6-chloro-7-phenyl-2-trifluoromethyl-2H-1- |
| | benzopyran-3-carboxylic acid; |
| B-45 | 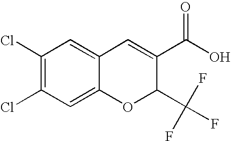 |
| | 6,7-dichloro-2-trifluoromethyl-2H-1- |
| | benzopyran-3-carboxylic acid; |
| B-46 | 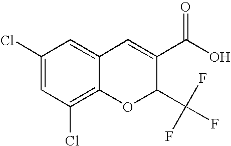 |
| | 6,8-dichloro-2-trifluoromethyl-2H-1- |
| | benzopyran-3-carboxylic acid; |
| B-47 | 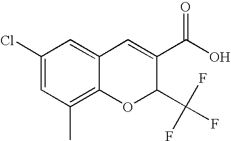 |
| | 6-chloro-8-methyl-2-trifluoromethyl-2H-1- |
| | benzopyran-3-carboxylic acid; |
| B-48 | 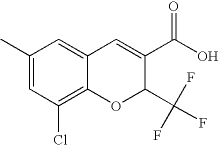 |
| | 8-chloro-6-methyl-2-trifluoromethyl-2H-1- |
| | benzopyran-3-carboxylic acid; |
| B-49 | 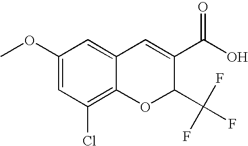 |
| | 8-chloro-6-methoxy-2-trifluoromethyl-2H-1- |
| | benzopyran-3-carboxylic acid; |
| B-50 | 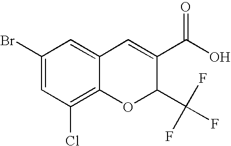 |
| | 6-bromo-8-chloro-2-trifluoromethyl-2H-1- |
| | benzopyran-3-carboxylic acid; |
| B-51 | 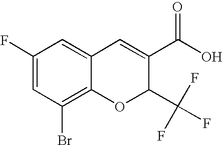 |
| | 8-bromo-6-fluoro-2-trifluoromethyl-2H-1- |
| | benzopyran-3-carboxylic acid; |
| B-52 | 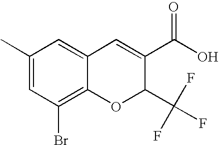 |
| | 8-bromo-6-methyl-2-trifluoromethyl-2H-1- |
| | benzopyran-3-carboxylic acid; |
| B-53 | 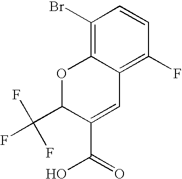 |
| | 8-bromo-5-fluoro-2-trifluoromethyl-2H-1- |
| | benzopyran-3-carboxylic acid; |
| B-54 | 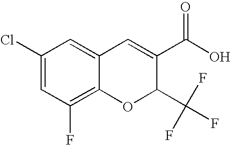 |
| | 6-chloro-8-fluoro-2-trifluoromethyl-2H-1- |
| | benzopyran-3-carboxylic acid; |
| B-55 | 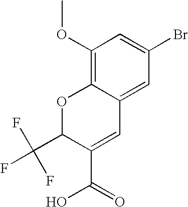 |
| | 6-bromo-8-methoxy-2-trifluoromethyl-2H-1- |
| | benzopyran-3-carboxylic acid; |
| B-56 | 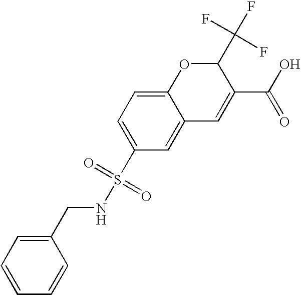 |
| | 6-[[(phenylmethyl)amino]sulfonyl]-2- |
| | trifluoromethyl-2H-1-benzopyran-3-carboxylic acid; |
| B-57 | 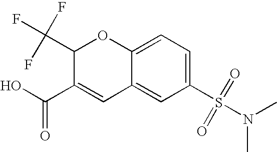 |
| | 6-[(dimethylamino)sulfonyl]-2-trifluoromethyl- |
| | 2H-1-benzopyran-3-carboxylic acid; |
| B-58 | 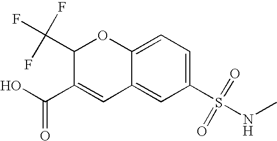 |
| | 6-[(methylamino)sulfonyl]-2-trifluoromethyl-2H-1- |
| | benzopyran-3-carboxylic acid; |
| B-59 | 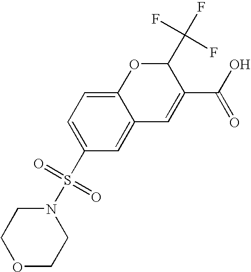 |
| | 6-[(4-morpholino)sulfonyl]-2-trifluoromethyl-2H- |
| | 1-benzopyran-3-carboxylic acid; |
| B-60 | 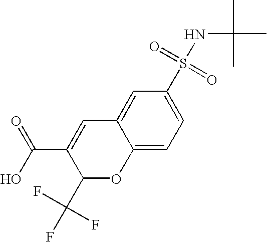 |
| | 6-[(1,1-dimethylethyl)aminosulfonyl]-2- |
| | trifluoromethyl-2H-1-benzopyran-3-carboxylic acid; |
| B-61 | 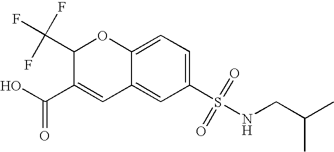 |
| | 6-[(2-methylpropyl)aminosulfonyl]-2- |
| | trifluoromethyl-2H-1-benzopyran-3-carboxylic acid; |
| B-62 | 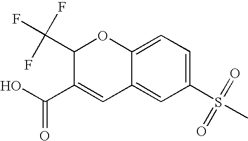 |
| | 6-methylsulfonyl-2-trifluoromethyl-2H-1- |
| | benzopyran-3-carboxylic acid; |
| B-63 | 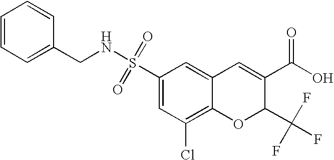 |
| | 8-chloro-6-[[(phenylmethyl)amino]sulfonyl]-2- |
| | trifluoromethyl-2H-1-benzopyran-3-carboxylic acid; |
| B-64 | 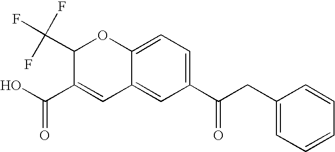 |
| | 6-phenylacetyl-2-trifluoromethyl-2H-1-benzopyran- |
| | 3-carboxylic acid; |
| B-65 | 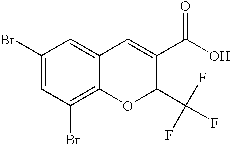 |
| | 6,8-dibromo-2-trifluoromethyl-2H-1-benzopyran- |
| | 3-carboxylic acid; |
| B-66 | 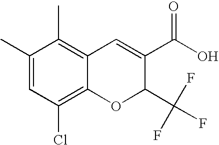 |
| | 8-chloro-5,6-dimethyl-2-trifluoromethyl-2H- |
| | 1-benzopyran-3-carboxylic acid; |
| B-67 | 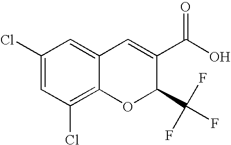 |
| | 6,8-dichloro-(S)-2-trifluoromethyl-2H-1- |
| | benzopyran-3-carboxylic acid; |
| B-68 | 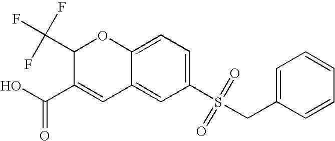 |
| | 6-benzylsulfonyl-2-trifluoromethyl-2H-1- |
| | benzopyran-3-carboxylic acid; |
| B-69 | 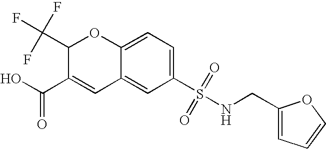 |
| | 6-[[N-(2-furylmethyl)amino]sulfonyl]-2- |
| | trifluoromethyl-2H-1-benzopyran-3-carboxylic acid; |
| B-70 | 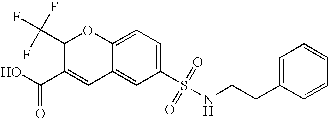 |
| | 6-[[N-(2-phenylethyl)amino]sulfonyl]-2- |
| | trifluoromethyl-2H-1-benzopyran-3- |
| | carboxylic acid; |
| B-71 | 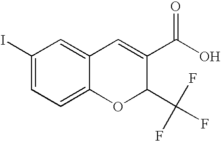 |
| | 6-iodo-2-trifluoromethyl-2H-1-benzopyran- |
| | 3-carboxylic acid; |
| B-72 | 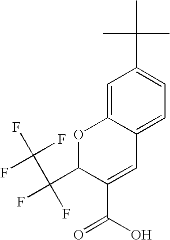 |
| | 7-(1,1-dimethylethyl)-2-pentafluoroethyl-2H- |
| | 1-benzopyran-3-carboxylic acid; |
| B-73 | 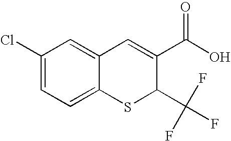 |
| | 6-chloro-2-trifluoromethyl-2H-1- |
| | benzothiopyran-3-carboxylic acid; |
| B-74 | 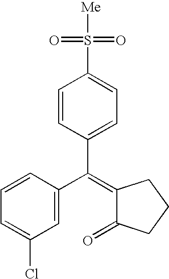 |
| | 3-[(3-chloro-phenyl)-(4-methanesulfonyl-phenyl)- |
| | methylene]-dihydro-furan-2-one or BMS-347070; |
| B-75 | 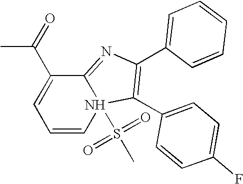 |
| | 8-acetyl-3-(4-fluorophenyl)-2-(4- |
| | methylsulfonyl)phenyl-imidazo(1,2-a)pyridine; |
| B-76 | 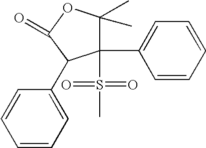 |
| | 5,5-dimethyl-4-(4-methylsulfanyl)phenyl-3- |
| | phenyl-2-(5H)-furanone; |
| B-77 | 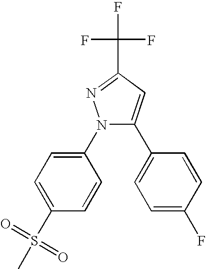 |
| | 5-(4-fluorophenyl)-1-[4-(methylsulfonyl)phenyl]- |
| | 3-(trifluoromethyl)pyrazole; |
| B-78 | 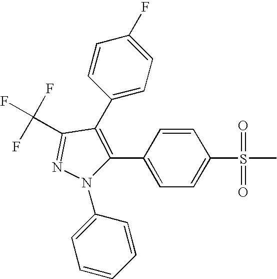 |
| | 4-(4-fluorophenyl)-5-[4-(methylsulfonyl)phenyl]- |
| | 1-phenyl-3-(trifluoromethyl)pyrazole; |
| B-79 | 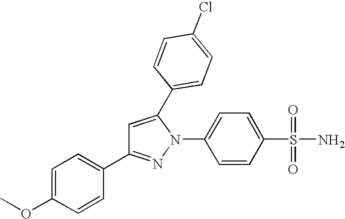 |
| | 4-(5-(4-chlorophenyl)-3-(4-methoxyphenyl)-1H- |
| | pyrazol-1-yl)benzenesulfonamide; |
| B-80 | 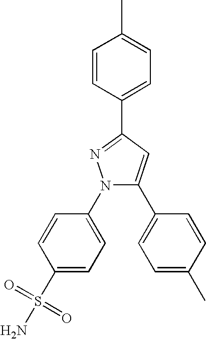 |
| | 4-(3,5-bis(4-methylphenyl)-1H-pyrazol-1- |
| | yl)benzenesulfonamide; |
| B-81 | 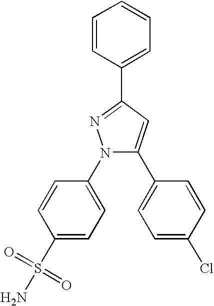 |
| | 4-(5-(4-chlorophenyl)-3-phenyl-1H-pyrazol-1- |
| | yl)benzenesulfonamide; |
| B-82 | 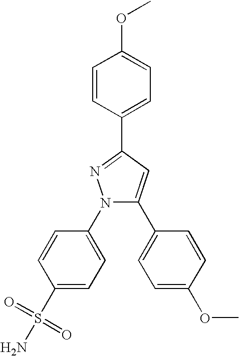 |
| | 4-(3,5-bis(4-methoxyphenyl)-1H-pyrazol-1- |
| | yl)benzenesulfonamide; |
| B-83 | 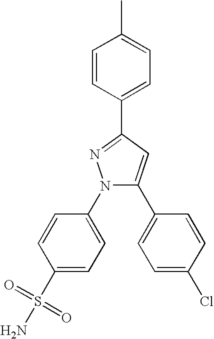 |
| | 4-(5-(4-chlorophenyl)-3-(4-methylphenyl)-1H- |
| | pyrazol-1-yl)benzenesulfonamide; |
| B-84 | 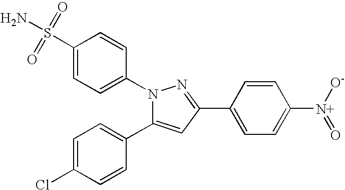 |
| | 4-(5-(4-chlorophenyl)-3-(4-nitrophenyl)-1H- |
| | pyrazol-1-yl)benzenesulfonamide; |
| B-85 | 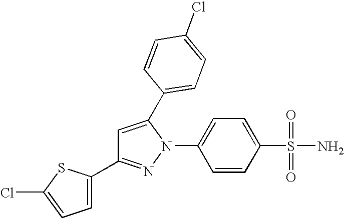 |
| | 4-(5-(4-chlorophenyl)-3-(5-chloro-2-thienyl)- |
| | 1H-pyrazol-1-yl)benzenesulfonamide; |
| B-86 | 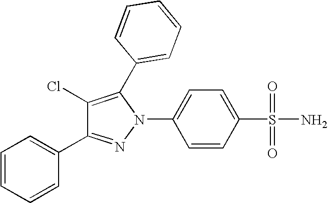 |
| | 4-(4-chloro-3,5-diphenyl-1H-pyrazol-1- |
| | yl)benzenesulfonamide; |
| B-87 | 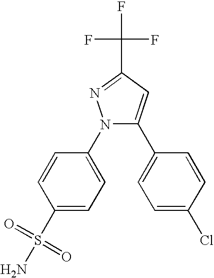 |
| | 4-[5-(4-chlorophenyl)-3-(trifluoromethyl)-1H- |
| | pyrazol-1-yl]benzenesulfonamide; |
| B-88 | 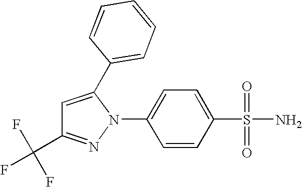 |
| | 4-[5-phenyl-3-(trifluoromethyl)-1H-pyrazol- |
| | 1-yl]benzenesulfonamide; |
| B-89 | 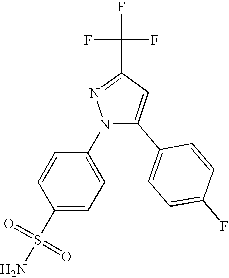 |
| | 4-[5-(4-fluorophenyl)-3-(trifluoromethyl)-1H- |
| | pyrazol-1-yl]benzenesulfonamide; |
| B-90 | 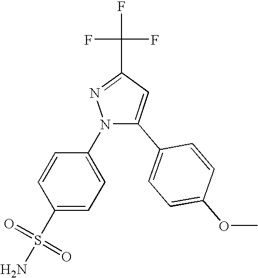 |
| | 4[5-(4-methoxyphenyl)-3-(trifluoromethyl)-1H- |
| | pyrazol-1-yl]benzenesulfonamide; |
| B-91 | 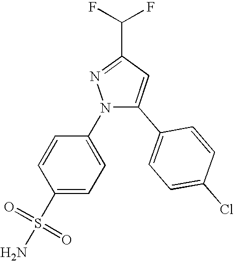 |
| | 4-[5-(4-chlorophenyl)-3-(difluoromethyl)-1H- |
| | pyrazol-1-yl]benzenesulfonamide; |
| B-92 | 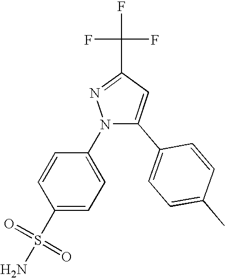 |
| | 4-[5-(4-methylphenyl)-3-(trifluoromethyl)-1H- |
| | pyrazol-1-yl]benzenesulfonamide; |
| B-93 | 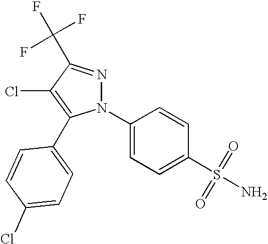 |
| | 4-[4-chloro-5-(4-chlorophenyl)-3- |
| | (trifluoromethyl)-1H-pyrazol-1-yl] |
| | benzenesulfonamide; |
| B-94 | 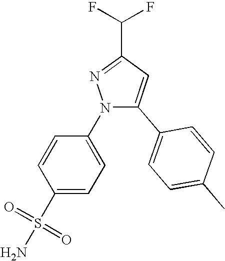 |
| | 4-[3-(difluoromethyl)-5-(4-methylphenyl)-1H- |
| | pyrazol-1-yl]benzenesulfonamide; |
| B-95 | 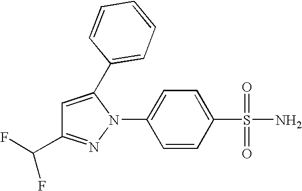 |
| | 4-[3-(difluoromethyl)-5-phenyl-1H-pyrazol-1- |
| | yl]benzenesulfonamide; |
| B-96 | 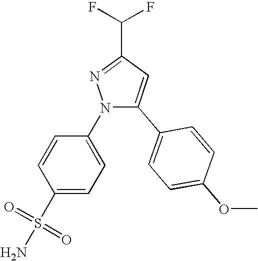 |
| | 4-[3-(difluoromethyl)-5-(4-methoxyphenyl)- |
| | 1H-pyrazol-1-yl]benzenesulfonamide; |
| B-97 | 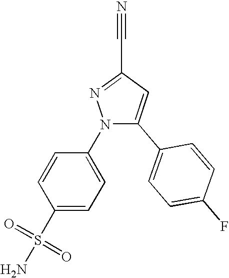 |
| | 4-[3-cyano-5-(4-fluorophenyl)-1H-pyrazol- |
| | 1-yl]benzenesulfonamide; |
| B-98 | 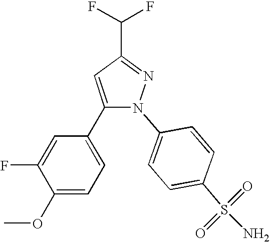 |
| | 4-[3-(difluoromethyl)-5-(3-fluoro-4- |
| | methoxyphenyl)-1H-pyrazol-1-yl] |
| | benzenesulfonamide; |

| Compound | |
|---|---|
| Number | Structural Formula |
| B-99 | 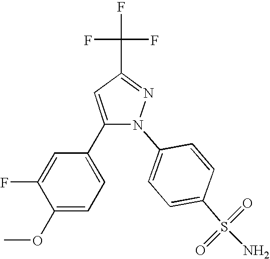 |
| | 4-[5-(3-fluoro-4-methoxyphenyl)-3-(trifluoromethyl)- |
| | 1H-pyrazol-1-yl]benzenesulfonamide; |
| B-100 | 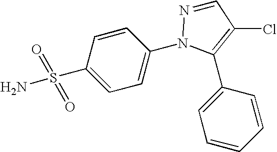 |
| | 4-[4-chloro-5-phenyl-1H-pyrazol-1-yl]benzenesulfonamide; |
| B-101 | 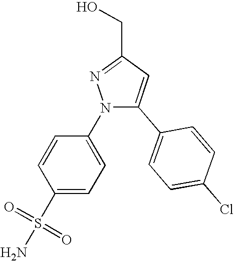 |
| | 4-[5-(4-chlorophenyl)-3-(hydroxymethyl)-1H- |
| | pyrazol-1-yl]benzenesulfonamide; |
| B-102 | 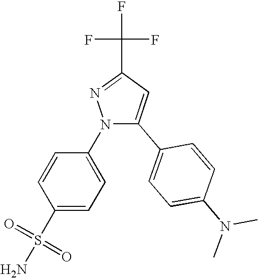 |
| | 4-[5-(4-(N,N-dimethylamino)phenyl)-3- |
| | (trifluoromethyl)-1H-pyrazol-1-yl]benzenesulfonamide; |
| B-103 | 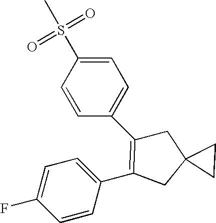 |
| | 5-(4-fluorophenyl)-6-[4-(methylsulfonyl)phenyl] |
| | spiro[2.4]hept-5-ene; |
| B-104 | 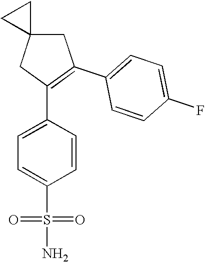 |
| | 4-[6-(4-fluorophenyl)spiro[2.4]hept-5-en-5- |
| | yl]benzenesulfonamide; |
| B-105 | 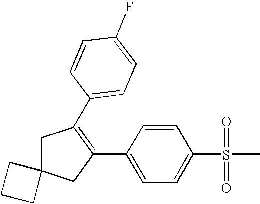 |
| | 6-(4-fluorophenyl)-7-[4-methylsulfonyl) |
| | phenyl]spiro[3.4]oct-6-ene; |
| B-106 | 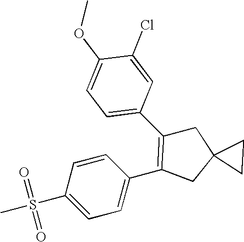 |
| | 5-(3-chloro-4-methoxyphenyl)-6-[4- |
| | (methylsulfonyl)phenyl]spiro[2.4]hept-5-ene; |
| B-107 | 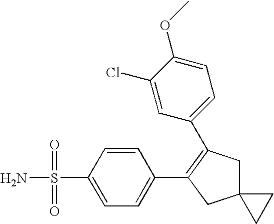 |
| | 4-[6-(3-chloro-4-methoxyphenyl)spiro[2.4]hept- |
| | 5-en-5-yl]benzenesulfonamide; |
| B-108 | 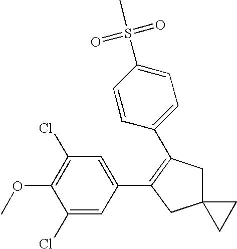 |
| | 5-(3,5-dichloro-4-methoxyphenyl)-6-[4- |
| | (methylsulfonyl)phenyl]spiro[2.4]hept-5-ene; |
| B-109 | 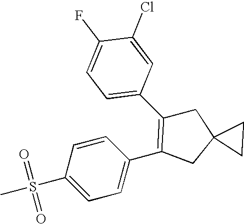 |
| | 5-(3-chloro-4-fluorophenyl)-6-[4- |
| | (methylsulfonyl)phenyl]spiro[2.4]hept-5-ene; |
| B-110 | 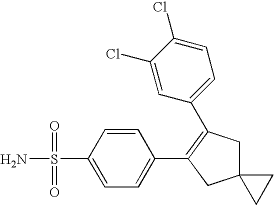 |
| | 4-[6-(3,4-dichlorophenyl)spiro[2.4]hept-5- |
| | en-5-yl]benzenesulfonamide; |
| B-111 | 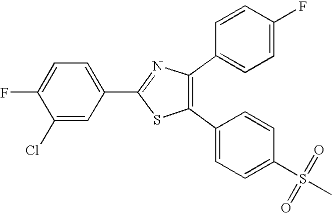 |
| | 2-(3-chloro-4-fluorophenyl)-4-(4-fluorophenyl)- |
| | 5-(4-methylsulfonylphenyl)thiazole; |
| B-112 | 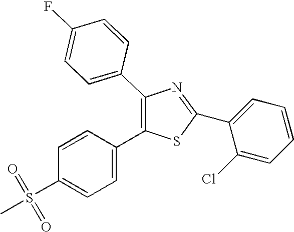 |
| | 2-(2-chlorophenyl)-4-(4-fluorophenyl)-5-(4- |
| | methylsulfonylphenyl)thiazole; |
| B-113 | 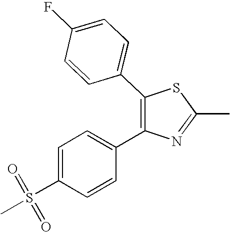 |
| | 5-(4-fluorophenyl)-4-(4-methylsulfonylphenyl)- |
| | 2-methylthiazole; |
| B-114 | 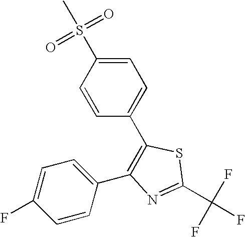 |
| | 4-(4-fluorophenyl)-5-(4-methylsulfonylphenyl)- |
| | 2-trifluoromethylthiazole; |
| B-115 | 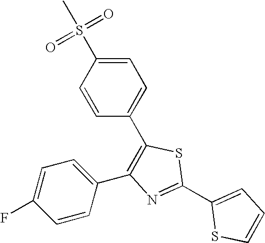 |
| | 4-(4-fluorophenyl)-5-(4-methylsulfonylphenyl)- |
| | 2-(2-thienyl)thiazole; |
| B-116 | 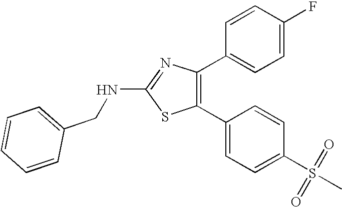 |
| | 4-(4-fluorophenyl)-5-(4-methylsulfonylphenyl)- |
| | 2-benzylaminothiazole; |
| B-117 | 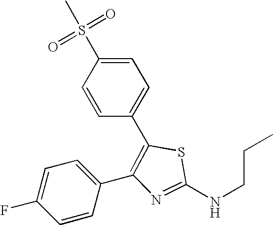 |
| | 4-(4-fluorophenyl)-5-(4-methylsulfonylphenyl)-2- |
| | (1-propylamino)thiazole; |
| B-118 | 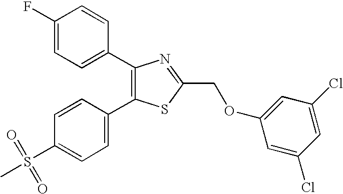 |
| | 2-((3,5-dichlorophenoxy)methyl)-4-(4- |
| | fluorophenyl)-5-[4-(methylsulfonyl)phenyl]thiazole; |
| B-119 | 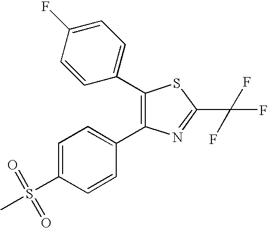 |
| | 5-(4-fluorophenyl)-4-(4-methylsulfonylphenyl)- |
| | 2-trifluoromethylthiazole; |
| B-120 | 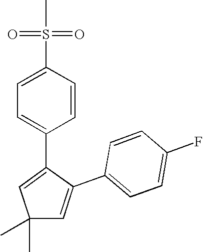 |
| | 1-methylsulfonyl-4-[1,1-dimethyl-4-(4-fluorophenyl) |
| | cyclopenta-2,4-dien-3-yl]bemzene; |
| B-121 | 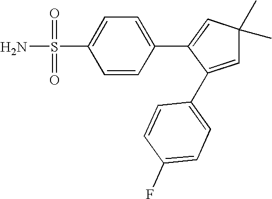 |
| | 4-[4-(4-fluorophenyl)-1,1-dimethylcyclopenta-2,4- |
| | dien-3-yl]benzenesulfonamide; |
| B-122 | 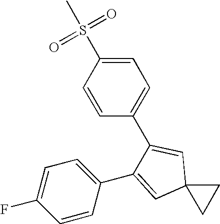 |
| | 5-(4-fluorophenyl)-6-[4-(methylsulfonyl)phenyl] |
| | spiro[2.4]hepta-4,6-diene; |
| B-123 | 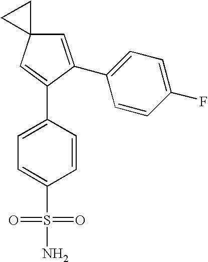 |
| | 4-[6-(4-fluorophenyl)spiro[2.4]hepta- |
| | 4,6-dien-5-yl]benzenesulfonamide; |
| B-124 | 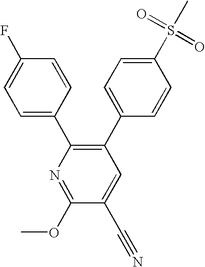 |
| | 6-(4-fluorophenyl)-2-methoxy-5-[4- |
| | (methylsulfonyl)phenyl]-pyridine-3-carbonitrile; |
| B-125 | 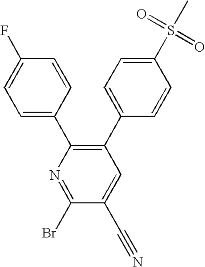 |
| | 2-bromo-6-(4-fluorophenyl)-5-[4- |
| | (methylsulfonyl)phenyl]-pyridine-3-carbonitrile; |
| B-126 | 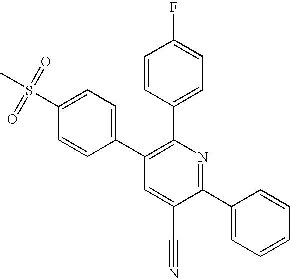 |
| | 6-(4-fluorophenyl)-5-[4-(methylsulfonyl)phenyl]- |
| | 2-phenyl-pyridine-3-carbonitrile; |
| B-127 | 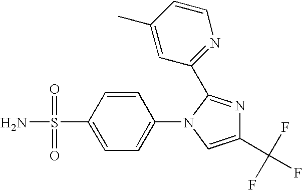 |
| | 4-[2-(4-methylpyridin-2-yl)-4-(trifluoromethyl)-1H- |
| | imidazol-1-yl]benzenesulfonamide; |
| B-128 | 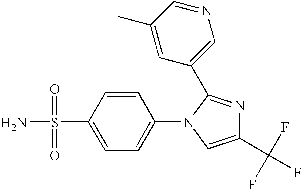 |
| | 4-[2-(5-methylpyridin-3-yl)-4-(trifluoromethyl)-1H- |
| | imidazol-1-yl]benzenesulfonamide; |
| B-129 | 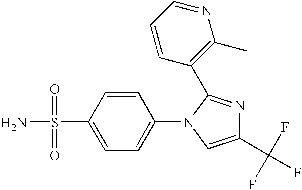 |
| | 4-[2-(2-methylpyridin-3-yl)-4-(trifluoromethyl)-1H- |
| | imidazol-1-yl]benzenesulfonamide; |
| B-130 | 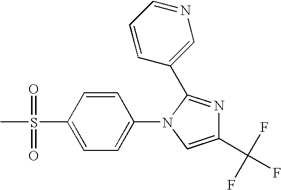 |
| | 3-[1-[4-(methylsulfonyl)phenyl]-4- |
| | (trifluoromethyl)-1H-imidazol-2-yl]pyridine; |
| B-131 | 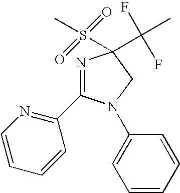 |
| | 2-[1-[4-(methylsulfonyl)phenyl-4- |
| | (trifluoromethyl)]-1H-imidazol-2-yl]pyridine; |
| B-132 | 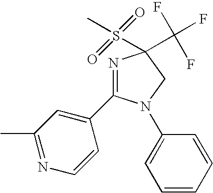 |
| | 2-methyl-4-[1-[4-(methylsulfonyl)phenyl- |
| | 4-(trifluoromethyl)]-1H-imidazol-2-yl]pyridine; |
| B-133 | 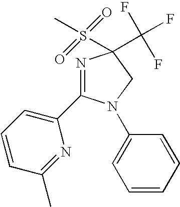 |
| | 2-methyl-6-[1-[4-(methylsulfonyl)phenyl-4- |
| | (trifluoromethyl)]-1H-imidazol-2-yl]pyridine; |
| B-134 | 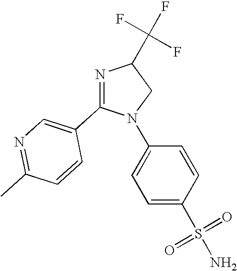 |
| | 4-[2-(6-methylpyridin-3-yl)-4-(trifluoromethyl)- |
| | 1H-imidazol-1-yl]benzenesulfonamide; |
| B-135 | 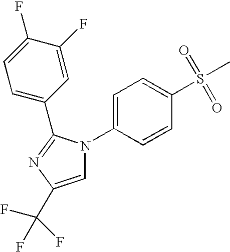 |
| | 2-(3,4-difluorophenyl)-1-[4-(methylsulfonyl)phenyl]- |
| | 4-(trifluoromethyl)-1H-imidazole; |
| B-136 | 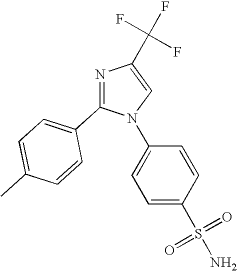 |
| | 4-(2-(4-methylphenyl)-4-(trifluorometbyl)-1H- |
| | imidazol-1-yl]benzenesulfonamide; |
| B-137 | 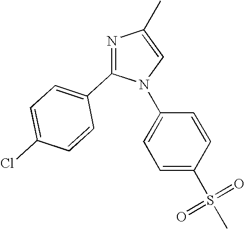 |
| | 2-(4-chlorophenyl)-1-[4-(methylsulfonyl)phenyl]- |
| | 4-methyl-1H-imidazole; |
| B-138 | 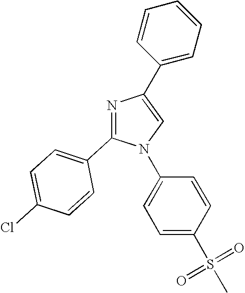 |
| | 2-(4-chlorophenyl)-1-[4-(methylsulfonyl)phenyl]- |
| | 4-phenyl-1H-imidazole; |
| B-139 | 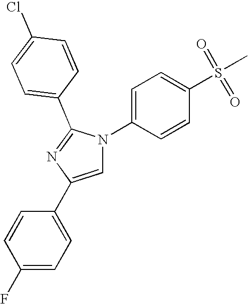 |
| | 2-(4-chlorophenyl)-4-(4-fluorophenyl)-1-[4- |
| | (methylsulfonyl)phenyl]-1H-imidazole; |
| B-140 | 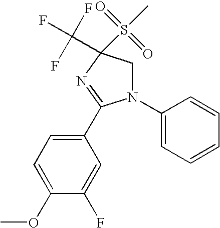 |
| | 2-(3-fluoro-4-methoxyphenyl)-1-[4- |
| | (methylsulfonyl)phenyl-4- |
| | (trifluoromethyl)]-1H-imidazole; |
| B-141 | 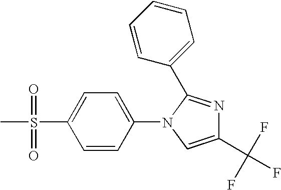 |
| | 1-[4-(methylsulfonyl)phenyl]-2-phenyl-4- |
| | trifluoromethyl-1H-imidazole; |
| B-142 | 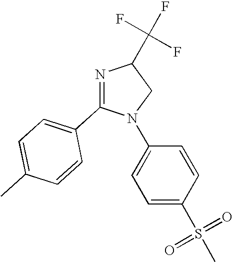 |
| | 2-(4-methylphenyl)-1-[4-(methylsulfonyl)phenyl]- |
| | 4-trifluoromethyl-1H-imidazole; |
| B-143 | 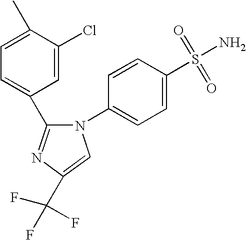 |
| | 4-[2-(3-chloro-4-methylphenyl)-4-(trifluoromethyl)- |
| | 1H-imidazol-1-yl]benzenesulfonamide; |
| B-144 | 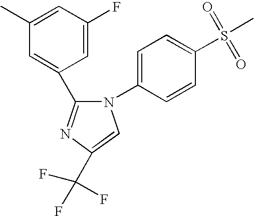 |
| | 2-(3-fluoro-5-methylphenyl)-1-[4-(methylsulfonyl) |
| | phenyl]-4-(trifluoromethyl)-1H-imidazole; |
| B-145 | 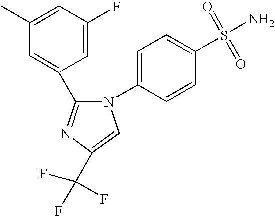 |
| | 4-[2-(3-fluoro-5-methylphenyl)-4-(trifluoromethyl- |
| | 1H-imidazole-1-yl]benzenesulfonamide; |
| B-146 | 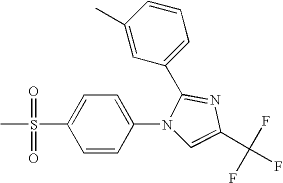 |
| | 2-(3-methylphenyl)-1-[4-(methylsulfonyl)phenyl]- |
| | 4-trifluoromethyl-1H-imidazole; |
| B-147 | 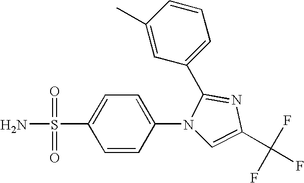 |
| | 4-[2-(3-methylphenyl)-4-trifluoromethyl-1H- |
| | imidazol-1-yl]benzenesulfonamide; |
| B-148 | 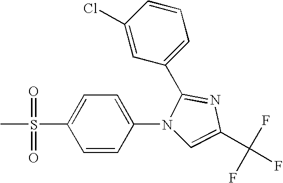 |
| | 1-[4-(methylsulfonyl)phenyl]-2-(3- |
| | chlorophenyl)-4-trifluoromethyl-1H-imidazole |
| B-149 | 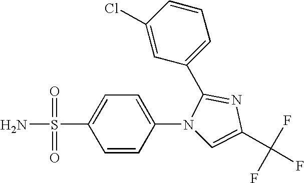 |
| | 4-[2-(3-chlorophenyl)-4-trifluoromethyl-1H- |
| | imidazol-1-yl]benzenesulfonamide; |
| B-150 | 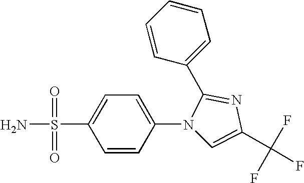 |
| | 4-[2-phenyl-4-trifluoromethyl-1H-imidazol-1- |
| | yl]benzenesulfonamide; |
| B-151 | 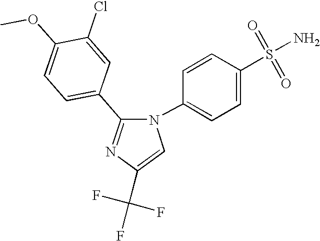 |
| | 4-[2-(4-methoxy-3-chlorophenyl)-4-trifluoromethyl- |
| | 1H-imidazol-1-yl]benzenesulfonamide; |
| B-152 | 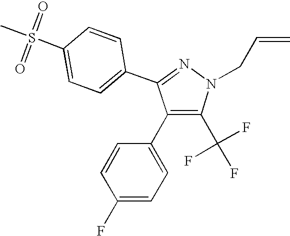 |
| | 1-allyl-4-(4-fluorophenyl)-3-[4-(methylsulfonyl) |
| | phenyl]-5-(trifluoromethyl)-1H-pyrazole; |
| B-153 | 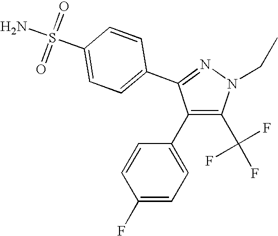 |
| | 4-[1-ethyl-4-(4-fluorophenyl)-5-(trifluoromethyl)- |
| | 1H-pyrazol-3-yl]benzenesulfonamide; |
| B-154 | 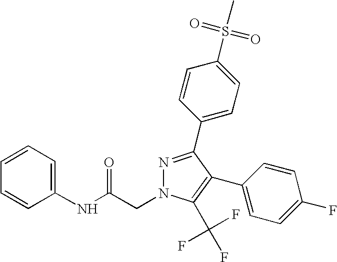 |
| | N-phenyl-[4-(4-fluorophenyl)-3-[4- |
| | (methylsulfonyl)phenyl]-5-(trifluoromethyl)- |
| | 1H-pyrazol-1-yl]acetamide; |
| B-155 | 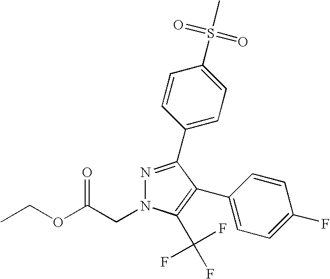 |
| | ethyl[4-(4-fluorophenyl)-3-[4- |
| | (methylsulfonyl)phenyl]-5-(trifluoromethyl)- |
| | 1H-pyrazol-1-yl]acetate; |
| B-156 | 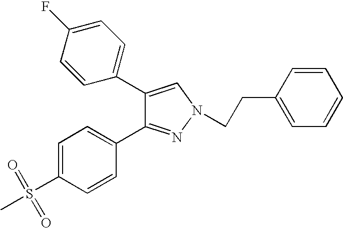 |
| | 4-(4-fluorophenyl)-3-[4-(methylsulfonyl)phenyl]- |
| | 1-(2-phenylethyl)-1H-pyrazole; |

| Compound | |
|---|---|
| Number | Structural Formula |
| B-157 | 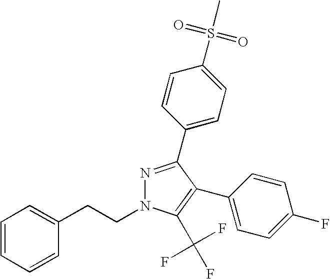 |
| | 4-(4-fluorophenyl)-3-[4-(methylsulfonyl)phenyl]- |
| | 1-(2-phenylethyl)-5-(trifluoromethyl)pyrazole; |
| B-158 | 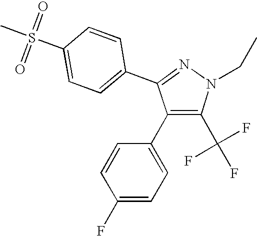 |
| | 1-ethyl-4-(4-fluorophenyl)-3-[4-methylsulfonyl) |
| | phenyl]-5-(trifluoromethyl)-1H-pyrazole; |
| B-159 | 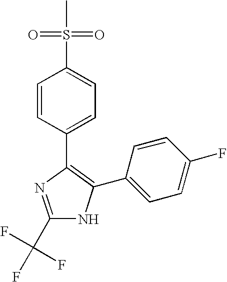 |
| | 5-(4-fluorophenyl)-4-(4-methylsulfonylphenyl)- |
| | 2-trifluoromethyl-1H-imidazole; |
| B-160 | 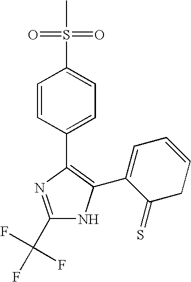 |
| | 4-[4-(methylsulfonyl)phenyl]-5-(2- |
| | thiophenyl)-2-(trifluoromethyl)-1H-imidazole; |
| B-161 | 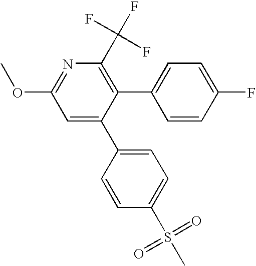 |
| | 5-(4-fluorophenyl)-2-methoxy-4-[4- |
| | (methylsulfonyl)phenyl]-6- |
| | (trifluoromethyl)pyridine; |
| B-162 | 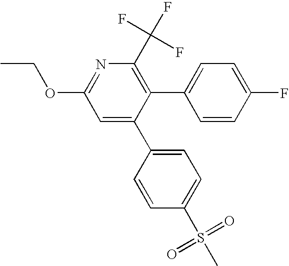 |
| | 2-ethoxy-5-(4-fluorophenyl)-4-[4- |
| | (methylsulfonyl)phenyl]-6- |
| | (trifluoromethyl)pyridine; |
| B-163 | 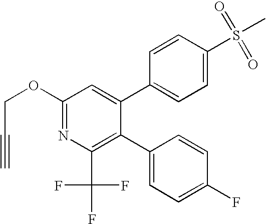 |
| | 5-(4-fluorophenyl)-4-[4-(methylsulfonyl) |
| | phenyl]-2-(2-propynyloxy)-6- |
| | (trifluoromethyl)pyridine; |
| B-164 | 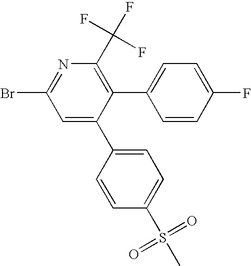 |
| | 2-bromo-5-(4-fluorophenyl)-4-[4-(methylsulfonyl) |
| | phenyl]-6-(trifluoromethyl)pyridine; |
| B-165 | 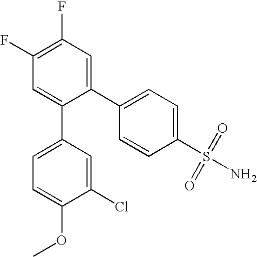 |
| | 4-[2-(3-chloro-4-methoxyphenyl)-4,5- |
| | difluorophenyl]benzenesulfonamide; |
| B-166 | 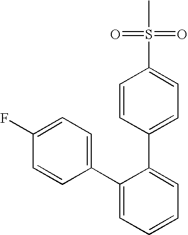 |
| | 1-(4-fluorophenyl)-2-[4-methylsulfonyl) |
| | phenyl]benzene; |
| B-167 | 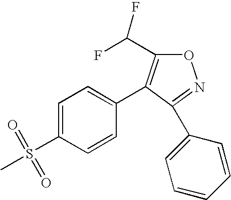 |
| | 5-difluoromethyl-4-(4-methylsulfonylphenyl)- |
| | 3-phenylisoxazole; |
| B-168 | 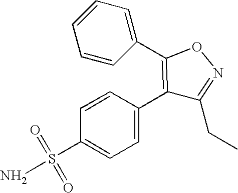 |
| | 4-[3-ethyl-5-phenylisoxazol-4- |
| | yl]benzenesulfonamide; |
| B-169 | 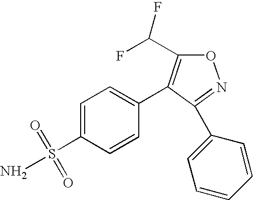 |
| | 4-[5-difluoromethyl-3-phenylisoxazol-4- |
| | yl]benzenesulfonamide; |
| B-170 | 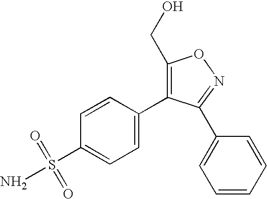 |
| | 4-[5-hydroxymethyl-3-phenylisoxazol-4- |
| | yl]benzenesulfonamide; |
| B-171 | 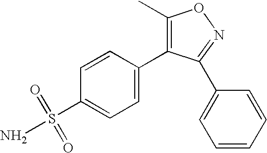 |
| | 4-[5-methyl-3-phenyl-isoxazol-4- |
| | yl]benzenesulfonamide; |
| B-172 | 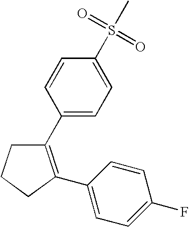 |
| | 1-[2-(4-fluorophenyl)cyclopenten-1-yl]- |
| | 4-(methylsulfonyl)benzene; |
| B-173 | 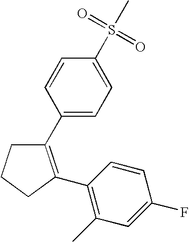 |
| | 1-[2-(4-fluoro-2-methylphenyl)cyclopenten- |
| | 1-yl]-4-(methylsulfonyl)benzene; |
| B-174 | 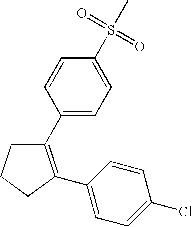 |
| | 1-[2-(4-chlorophenyl)cyclopenten-1- |
| | yl]-4-(methylsulfonyl)benzene; |
| B-175 | 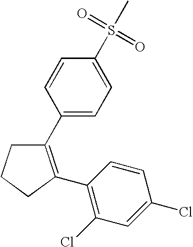 |
| | 1-[2-(2,4-dichlorophenyl)cyclopenten-1- |
| | yl]-4-(methylsulfonyl)benzene; |
| B-176 | 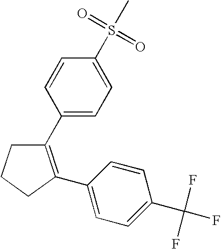 |
| | 1-[2-(4-trifloromethylphenyl)cyclopenten-1- |
| | yl]-4-(methylsulfonyl)benzene; |
| B-177 | 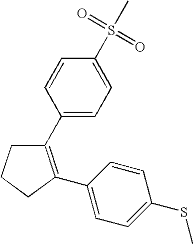 |
| | 1-[2-(4-methylthiophenyl)cyclopenten-1- |
| | yl]-4-(methylsulfonyl)benzene; |
| B-178 | 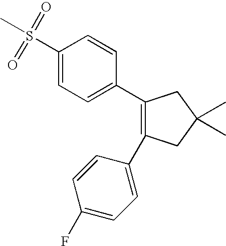 |
| | 1-[2-(4-fluorophenyl)-4,4-dimethylcyclopenten- |
| | 1-yl]-4-(methylsulfonyl)benzene; |
| B-179 | 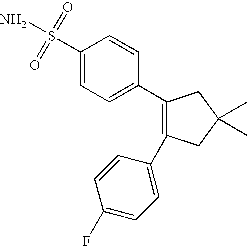 |
| | 4-[2-(4-fluorophenyl)-4,4-dimethylcyclopenten-1- |
| | yl]benzenesulfonamide; |
| B-180 | 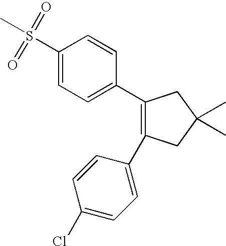 |
| | 1-[2-(3-chlorophenyl)-4,4-dimethylcyclopenten-1- |
| | yl]-4-(methylsulfonyl)benzene; |
| B-181 | 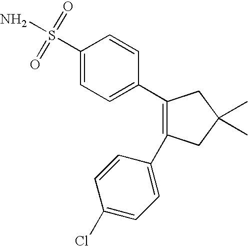 |
| | 4-[2-(4-chlorophenyl)-4,4-dimethylcyclopenten-1- |
| | yl]benzenesulfonamide; |
| B-182 | 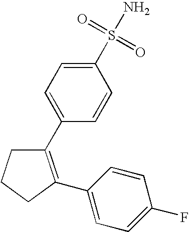 |
| | 4-[2-(4-fluorophenyl)cyclopenten-1- |
| | yl]benzenesulfonamide; |
| B-183 | 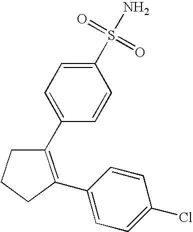 |
| | 4-[2-(4-chlorophenyl)cyclopenten-1- |
| | yl]benzenesulfonamide; |
| B-184 | 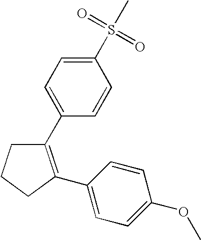 |
| | 1-[2-(4-methoxyphenyl)cyclopenten-1-yl]-4- |
| | (methylsulfonyl)benzene; |
| B-185 | 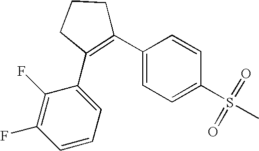 |
| | 1-[2-(2,3-difluorophenyl)cyclopenten-1-yl]- |
| | 4-(methylsulfonyl)benzene; |
| B-186 | 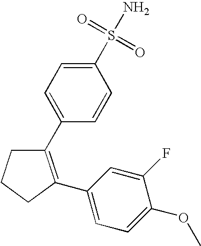 |
| | 4-[2-(3-fluoro-4-methoxyphenyl)cyclopenten- |
| | 1-yl]benzenesulfonamide; |
| B-187 | 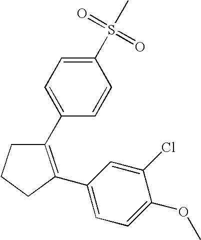 |
| | 1-[2-(3-chloro-4-methoxyphenyl)cyclopenten-1- |
| | yl]-4-(methylsulfonyl)benzene; |
| B-188 | 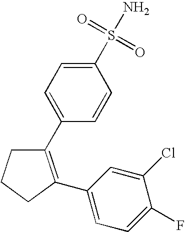 |
| | 4-[2-(3-chloro-4-fluorophenyl)cyclopenten-1- |
| | yl]benzenesulfonamide; |
| B-189 | 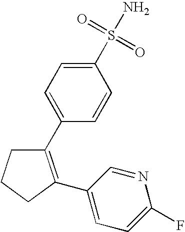 |
| | 4-[2-(2-methylpyridin-5-yl)cyclopenten-1- |
| | yl]benzenesulfonamide; |
| B-190 | 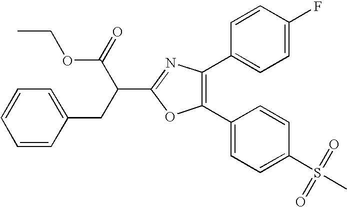 |
| | ethyl 2-[4-(4-fluorophenyl)-5-[4- |
| | (methylsulfonyl)phenyl]oxazol-2-yl]-2- |
| | benzyl-acetate; |
| B-191 | 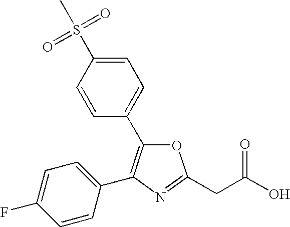 |
| | 2-[4-(4-fluorophenyl)-5-[4-(methylsulfonyl) |
| | phenyl]oxazol-2-yl]acetic acid; |
| B-192 | 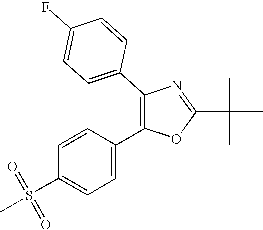 |
| | 2-(tert-butyl)-4-(4-fluorophenyl)-5-[4- |
| | (methylsulfonyl)phenyl]oxazole; |
| B-193 | 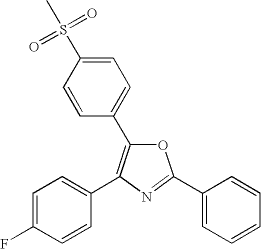 |
| | 4-(4-fluorophenyl)-5-[4-(methylsulfonyl) |
| | phenyl]-2-phenyloxazole; |
| B-194 | 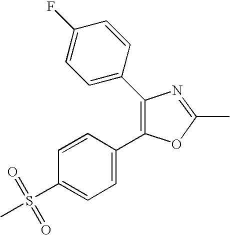 |
| | 4-(4-fluorophenyl)-2-methyl-5-[4- |
| | (methylsulfonyl)phenyl]oxazole; |
| B-195 | 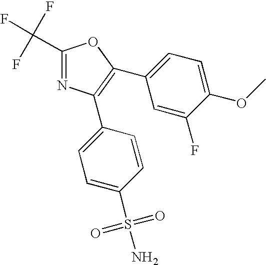 |
| | 4-[5 -(3-fluoro-4-methoxyphenyl)-2- |
| | trifluoromethyl-4-oxazolyl]benzenesulfonamide; |
| B-196 | 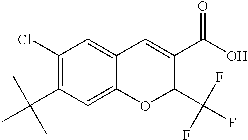 |
| | 6-chloro-7-(1,1-dimethylethyl)-2-trifluoromethyl- |
| | 2H-1-benzopyran-3-carboxylic acid; |
| B-197 | 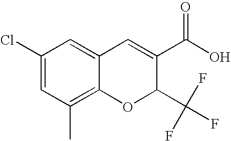 |
| | 6-chloro-8-methyl-2-trifluoromethyl-2H-1- |
| | benzopyran-3-carboxylic acid; |
| B-198 | 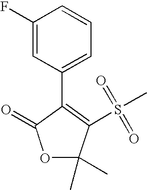 |
| | 5,5-dimethyl-3-(3-fluorophenyl)-4-methylsulfonyl- |
| | 2(5H)-furanone; |
| B-199 | 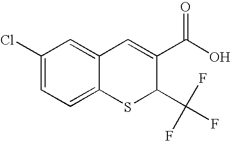 |
| | 6-chloro-2-trifluoromethyl-2H-1- |
| | benzothiopyran-3-carboxylic acid; |
| B-200 | 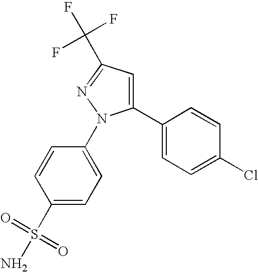 |
| | 4-[5-(4-chlorophenyl)-3-(trifluoromethyl)-1H- |
| | pyrazol-1-yl]benzenesulfonamide; |
| B-201 | 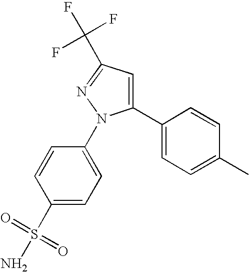 |
| | 4-[5-(4-methylphenyl)-3-(trifluoromethyl)-1H- |
| | pyrazol-1-yl]benzenesulfonamide; |
| B-202 | 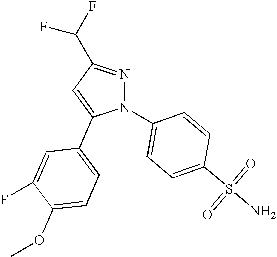 |
| | 4-[5-(3-fluoro-4-methoxyphenyl)-3- |
| | (difluoromethyl)-1H-pyrazol-1-yl]benzenesulfonamide; |
| B-203 | 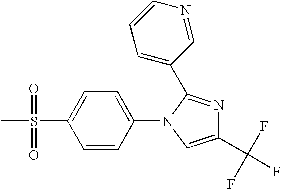 |
| | 3-[1-[4-(methylsulfonyl)phenyl]-4- |
| | trifluoromethyl-1H-imidazol-2-yl]pyridine; |
| B-204 | 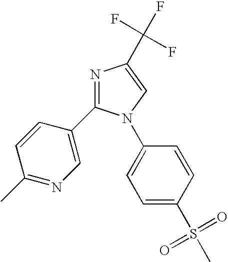 |
| | 2-methyl-5-[1-[4-(methylsulfonyl)phenyl]-4- |
| | trifluoromethyl-1H-imidazol-2-yl]pyridine; |
| B-205 | 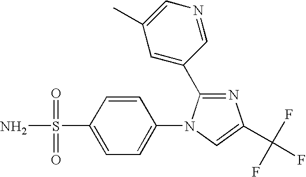 |
| | 4-[2-(5-methylpyridin-3-yl)-4-(trifluoromethyl)- |
| | 1H-imidazol-1-yl]benzenesulfonamide; |
| B-206 | 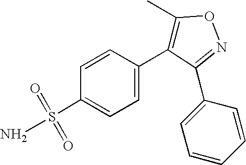 |
| | 4-[5-methyl-3-phenylisoxazol-4-yl]benzenesulfonamide; |
| B-207 | 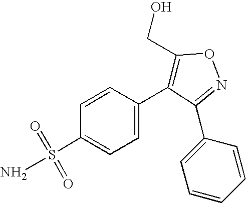 |
| | 4-[5-hydroxymethyl-3-phenylisoxazol-4- |
| | yl]benzenesulfonamide; |
| B-208 | 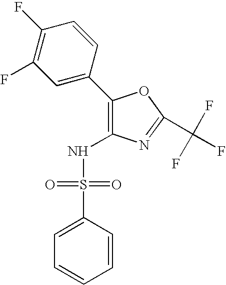 |
| | [2-trifluoromethyl-5-(3,4-difluorophenyl)-4- |
| | oxazolyl]benzenesulfonamide; |
| B-209 | 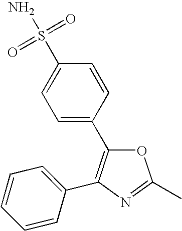 |
| | 4-[2-methyl-4-phenyl-5- |
| | oxazolyl]benzenesulfonamide; |
| B-210 | 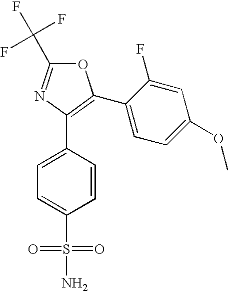 |
| | 4-[5-(2-fluoro-4-methoxyphenyl)-2- |
| | trifluoromethyl-4-oxazolyl]benzenesulfonamide; |
| B-211 | 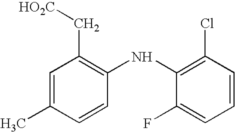 |
| B-212 | 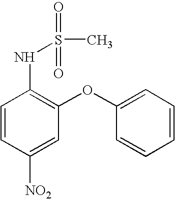 |
| | N-(4-nitro-2-phenoxy-phenyl)- |
| | methanesulfonamide or Nimesulide |

| Compound | |
|---|---|
| Number | Structural Formula |
| B-213 | 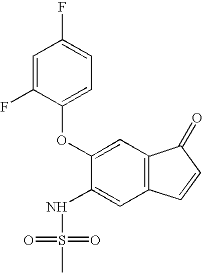 |
| | N-[6-(2,4-difluoro-phenoxy)-1-oxo-inden-5-yl]- |
| | methanesulfonamide or Flosulide |
| B-214 | 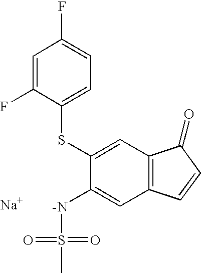 |
| | N-[6-(2,4-difluoro-phenylsulfanyl)-1-oxo-1H-inden- |
| | 5-yl]-methanesulfonamide, soldium salt, or |
| | L-745337 |
| B-215 | 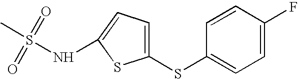 |
| | N-[5-(4-fluoro-phenylsulfanyl)-thiophen-2-yl]- |
| | methanesulfonamide or RWJ-63556 |
| B-216 | 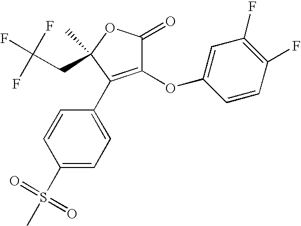 |
| | 3-(3,4-difluoro-phenoxy)-4-(4-methanesulfonyl-phenyl)- |
| | 5-methyl-5-(2,2,2-trifluoro-ethyl)-5H-furan-2-one or |
| | L-784512 |
| B-217 | 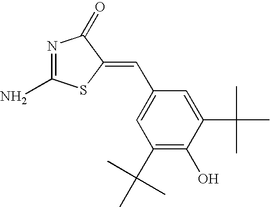 |
| | (5Z)-2-amino-5-[[3,5-bis(1,1-dimethylethyl)-4- |
| | hydroxyphenyl]methylene]-4(5H)-thiazolone or |
| | Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 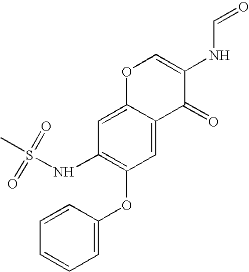 |
| | N-[3-(formylamino)-4-oxo-6-phenoxy-4H-1- |
| | benzopyran-7-yl]-methanesulfonamide or T614 |
| B-225 | D-1367 |
| B-226 | L-748731 |
| B-227 | 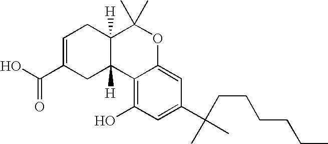 |
| | (6aR,10aR)-3-(1,1-dimethylheptyl)-6a,7,10,10a-tetrahydro- |
| | 1-hydroxy-6,6-dimethyl-6H-dibenzo[b,d]pyran-9- |
| | carboxylic acid or CT3 |
| B-228 | CGP-28238 |
| B-229 | 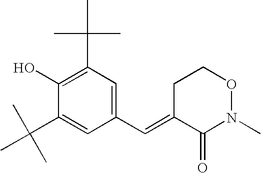 |
| | 4-[[3,5-bis(1,1-dimethylethyl)-4- |
| | hydroxyphenyl]methylene]dihydro-2-methyl- |
| | 2H-1,2-oxazin-3(4H)-one or BF-389 |
| B-230 | GR-253035 |
| B-231 | 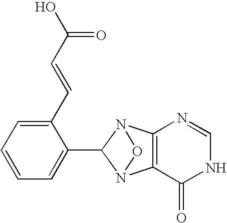 |
| | 2-(6-dioxo-9H-purin-8-yl)cinnamic acid |
| B-232 | S-2474 |
| B-233 | 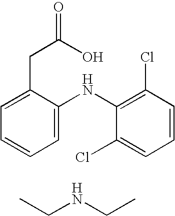 |
| B-234 | 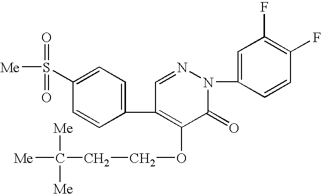 |
| B-235 | 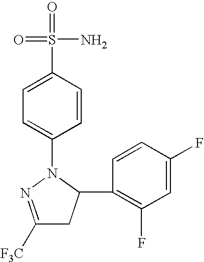 |
| B-236 | 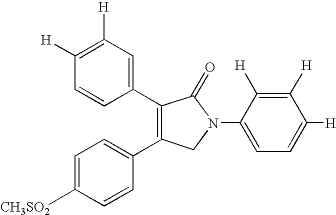 |
| B-237 | 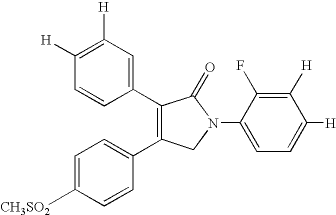 |
| B-238 | 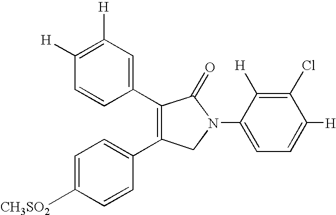 |
| B-239 | 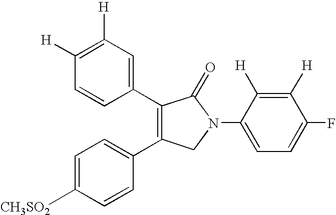 |
| B-240 | 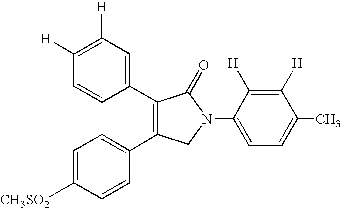 |
| B-241 | 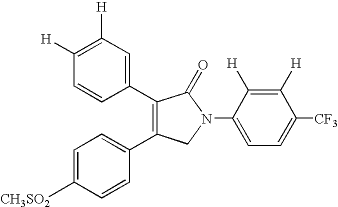 |
| B-242 | 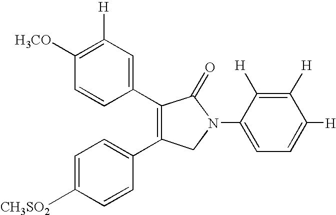 |
| B-243 | 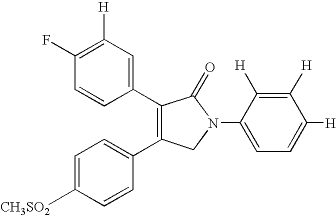 |
| B-244 | 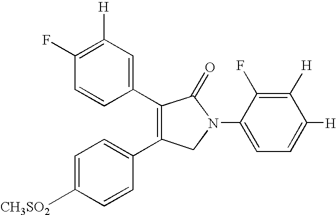 |
| B-245 | 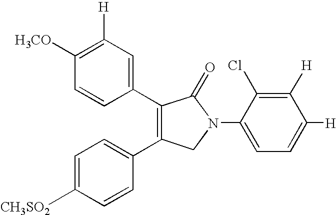 |
| B-246 | 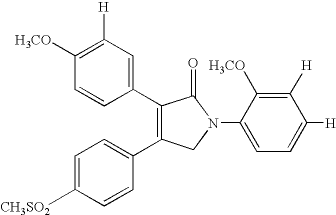 |
| B-247 | 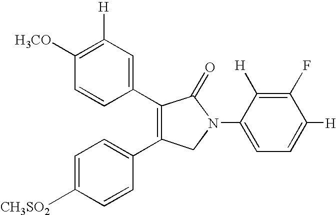 |
| B-248 | 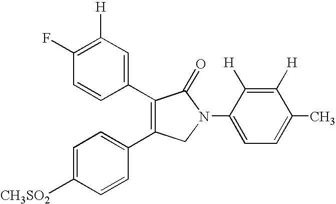 |
| B-249 | 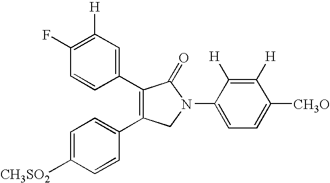 |
| B-250 | 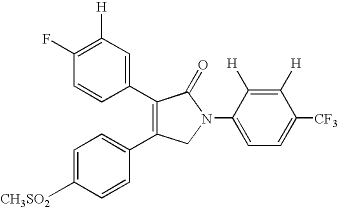 |
| B-251 | 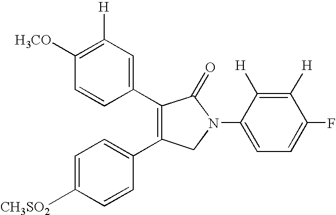 |
| B-252 | 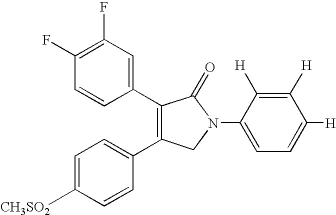 |

The cyclooxygenase-2 selective inhibitor employed in the present invention can exist in tautomeric, geometric or stereoisomeric forms. Generally speaking, suitable cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R- and S-enantiomers, diastereomers, d-isomers, 1-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

The cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors of the present invention can be formulated into pharmaceutical compositions and administered by a number of different means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose is generally administered in one to about four doses per day.

In one embodiment, when the cyclooxygenase-2 selective inhibitor comprises rofecoxib, it is typical that the amount used is within a range of from about 0.15 to about 1.0 mg/day·kg, and even more typically, from about 0.18 to about 0.4 mg/day·kg.

In still another embodiment, when the cyclooxygenase-2 selective inhibitor comprises etoricoxib, it is typical that the amount used is within a range of from about 0.5 to about 5 mg/day·kg, and even more typically, from about 0.8 to about 4 mg/day·kg.

Further, when the cyclooxygenase-2 selective inhibitor comprises celecoxib, it is typical that the amount used is within a range of from about 1 to about 20 mg/day·kg, even more typically, from about 1.4 to about 8.6 mg/day·kg, and yet more typically, from about 2 to about 3 mg/day·kg.

When the cyclooxygenase-2 selective inhibitor comprises valdecoxib, it is typical that the amount used is within a range of from about 0.1 to about 5 mg/day·kg, and even more typically, from about 0.8 to about 4 mg/day·kg.

In a further embodiment, when the cyclooxygenase-2 selective inhibitor comprises parecoxib, it is typical that the amount used is within a range of from about 0.1 to about 5 mg/day·kg, and even more typically, from about 1 to about 3 mg/day·kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

PPAR Agonists

In addition to a cyclooxygenase-2 selective inhibitor, the composition of the invention also includes a PPAR agonist. A number of different PPAR agonists may be employed in the present invention. In some embodiments, the PPAR agonist is an agonist of a PPARα. In other embodiments, the PPAR agonist is an agonist of a PPARγ. In still other embodiments, the PPAR agonist is an agonist of a PPARβ/δ. In still further embodiments, the PPAR agonist may be an agonist of one or more of the PPARα, PPARγ, or PPARβ/δ.

One aspect of the invention comprises PPAR agonists that are members of the thiazolidinedione class of compounds. In one embodiment, the thiazolidinedione is a thiazolidine 2-4 dione. In one alternative of this embodiment, the thiazolidine 2-4 dione is a compound having formula (X) 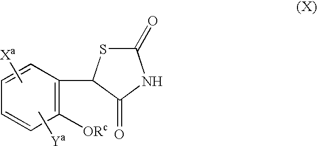
wherein:

R^{c} is lower alkyl;
X^{a} is fluoro, chloro, or bromo; and
Y^{a} is hydrogen, chloro, lower alkyl or lower alkoxy.

Other suitable compounds corresponding to formula (X) are detailed in U.S. Pat. No. 4,617,312, which is hereby incorporated by reference in its entirety.

In another alternative of this embodiment, the thiazolidine 2-4 dione is a compound having formula (XI) 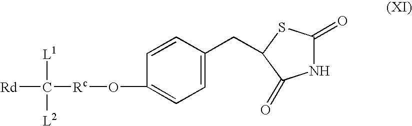
wherein:
R^{c} is a bond or lower alkylene;
R^{d} is an optionally substituted five- or six-membered heterocyclic group including one or two hetero-atoms selected from N, O and S; and
L¹ and L² are independently hydrogen or lower alkyl.

Examples of other suitable compounds corresponding to formula (XI) are disclosed in U.S. Pat. No. 4,340,605, which is hereby incorporated by reference in its entirety.

In still another alternative of this embodiment, the thiazolidine 2-4 dione is a compound having formula (XII) 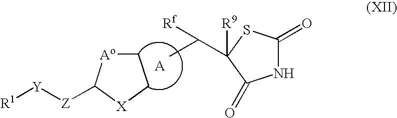
wherein:
A^{o} is —NH— or a moiety R^{h}—C—, where R^{h} is hydrogen, alkyl, or optionally substituted aryl;
R^{j} is optionally substituted phenyl, optionally substituted pyridyl, or optionally substituted oxazolyl;
Y is —(CH₂)ₙ—, where n is 0 to 6;
Z is —CH₂—, —CH(OH)—, or —CO—;
R^{f} and R^{g} are independently hydrogen or R^{f} and R^{g} together represent a bond;
A is a residue of a benzene ring, the carbon atoms of the residue having in total up to four substituents; and
X is oxygen or sulfur.

In another alternative of this embodiment, the thiazolidine 2-4 dione is a compound having formula (XIII) 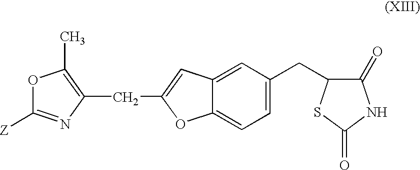
wherein Z is an alkyl having one to six carbon atoms, a cycloalkyl having three to seven carbon atoms, a phenyl or mono- or di-substituted phenyl wherein said substituents are independently an alkyl having one to six carbon atoms, an alkoxy having one to three carbon atoms, halogen or trifluoromethyl.

In one alternative embodiment for compounds having formula (XIII), Z is (C₁-C₆) alkyl, (C₃-C₇) cycloalkyl, phenyl, halophenyl or (C₁-C₆) alkylphenyl. In another alternative embodiment for compounds having formula (XIII), Z is phenyl, methylphenyl, fluorophenyl, chlorophenyl or cyclohexyl. Examples of other suitable compounds having formula (XIII) are disclosed in U.S. Pat. No. 5,401,761, which is hereby incorporated by reference in its entirety.

In yet another alternative of this embodiment, the thiazolidine 2-4 dione is a compound having formula (XIV) 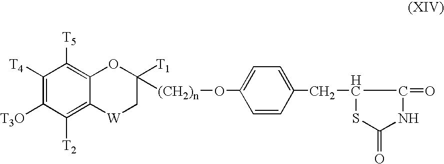
wherein:
T₁ and T₂ are independently hydrogen or C₁-C₅ alkyl;
T₃ is hydrogen, C₁-C₆ aliphatic acyl, alicyclic acyl, aromatic acyl, heterocyclic acyl, araliphatic acyl, (C₁-C₆ alkoxy)carbonyl, or aralkyloxycarbonyl;
T₄ and T₅ are independently hydrogen, C₁-C₅ alkyl, C₁-C₅ alkoxy, or T₄ and T₅ form a alkylenedioxy ring having 1 to 4 carbon atoms;
n is 1, 2, or 3;
W is methylene, CO, or CH—OT₆; and
T₆ is hydrogen, C₁-C₆ aliphatic acyl, alicyclic acyl, aromatic acyl, heterocyclic acyl, araliphatic acyl, (C₁-C₆ alkoxy)carbonyl, or aralkyloxycarbonyl.

In one alternative embodiment for compounds having formula (XIV), the compound is a 5-[4-(chromoanalkoxy)benzyl]thiazolidene derivative, having any of XIV(a), XIV(b), and XIV(c) 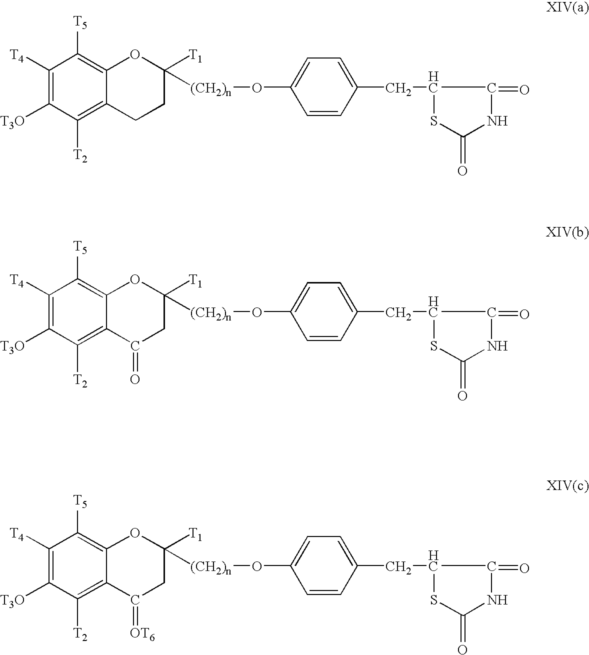
wherein T₁, T₂, T₃, T₄, T₅, T₆, and n are as defined for compounds having formula (XIV).

In a further alternative embodiment, the compound having formula (XIV) is selected from the group consisting of:
(+)-5-[[4-[(3,4-dihydro-6-hydroxy-2,5,7,8-tetramethyl-2H-1-benzopyran-2-yl)methoxy]phenyl]methyl]-2,4-thiazolidinedione;
4-(2-naphthylmethyl)-1,2,3,5-oxathiadiazole-2-oxide;
5-[4-[2-[N-(benzoxazol-2-yl)-N-methylamino]ethoxy]benzyl]5-methylthiazolidine-2,4-dione;
5-[4-[2-[2,4-dioxo-5-phenylthiazolidin-3-yl)ethoxy]benzyl]thiazolidine-2,4-dione;
5-[4-[2-[N-methyl-N-(phenoxycarbonyl)amino]ethoxy]benzyl]thiazolidine-2,4-dione;
5-[4-(2-phenoxyethoxy)benzyl]thiazolidine-2,4-dione;
5-[4-[3-(5-methyl-2-phenyloxazol-4-yl)propionyl]benzyl]thiazolidine-2,4-dione;
5-[4-[2-(4-chlorophenyl)ethylsulfonyl]benzyl]thiazolidine-2,4-dione;
5-[4-[3-(5-methyl-2-phenyloxazol-4-yl)propionyl]benzyl]thiazolidine-2,4-dione;
5-(4-[2-(N-methyl-N-(2-pyridyl)amino)-ethoxy]benzyl)2,4-thiazolidinedione;
5-[p-[1-methylcyclohexyl)methoxyl]benzyl]-2,4-thiazolidinedione (Ciglitazone);
5-[p-[2-(5-ethyl-2-pyridyl)ethoxy]benzyl]-2,4-thiazolidinedione (Pioglitazone);
5-[p-[3-(5-methyl-2-phenyl-4-oxazolyl)propionyl]benzyl]-2,4-thiazolidinedione (Darglitazone);
5-[[(2R)-2-benzyl-6-chromanyl]methyl]-2,4-thiazolidinedione (Englitazone); and
5-(4-[2-(N-methyl-N-(2-pyridyl)amino)ethoxy]benzyl)-2,4-thiazolidinedione (Rosiglitazone).

Still other suitable examples of compounds having formula (XIV) are disclosed in U.S. Pat. No. 6,130,216, which is hereby incorporated by reference it its entirety.

In another alternative of this embodiment, the thiazolidine 2-4 dione is a compound having formula (XV) 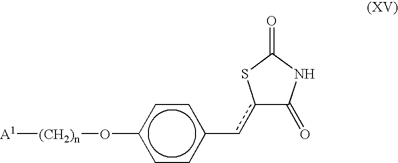
wherein:
n stands for an integer of 1 to 4; 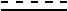
is a single or double bond; and
A¹ is a heterocycle selected from the group consisting of: 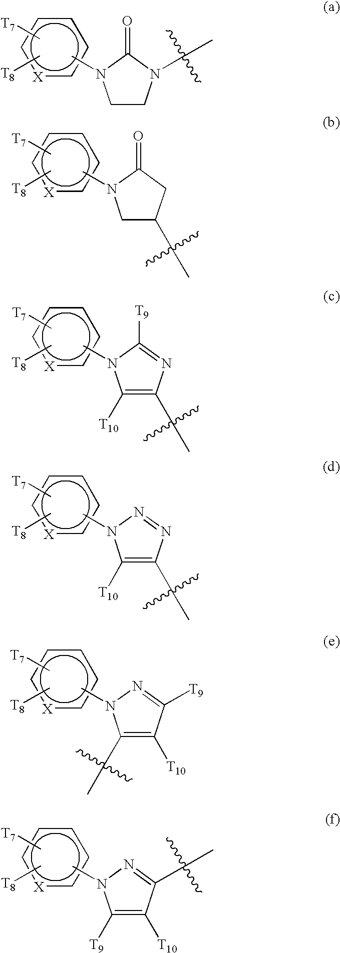
wherein:
X is NH or CH₂;
T₇ and T₈ are independently hydrogen, halogen, halogen substituted or unsubstituted lower alkyl, halogen substituted or unsubstituted lower alkoxyl, or T₇ and T₈ form a alkylenedioxy ring having 1 to 3 carbon atoms; and
T₉ and T₁₀ are independently hydrogen or lower alkyl.

Examples of other suitable compounds having formula (XV) are disclosed in U.S. Pat. No. 5,990,139, which is hereby incorporated by reference in its entirety.

In yet another alternative of this embodiment, the thiazolidine 2-4 dione is a compound having formula (XVI) 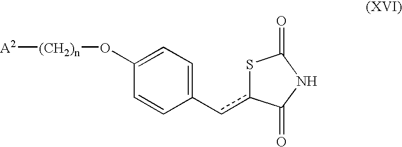
wherein:
n represents an integer from 1 to 3;
A² represents an aromatic 5-membered heterocyclic ring residue which has at least one nitrogen atom as a ring atom, which is bound via a carbon atom adjacent to the nitrogen atom and which may be substituted; and

[symbol] is a single or double bond.

Examples of other suitable compounds having formula (XVI) are detailed in U.S. Pat. No. 5,489,602, which is hereby incorporated by reference in its entirety.

In still another alternative of this embodiment, the thiazolidine 2-4 dione is a compound having formula (XVII) 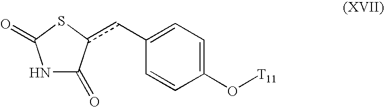
wherein: 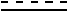
is a single or double bond;
T₁₁ is: 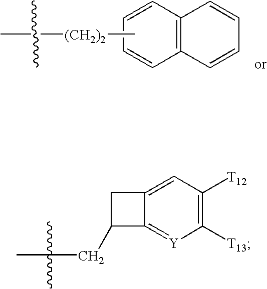
Y is CH; and
T₁₂ and T₁₃ are independently hydrogen, halogen, linear or branched (C₁-C₆) alkoxy, or together form methylenedioxy or ethylenedioxy.

Other suitable compounds having formula (XVII) are disclosed in U.S. Pat. No. 5,330,999, which is hereby incorporated by reference in its entirety.

Examples of other suitable thiazolidinediones are shown in Table 4.

TABLE 4

| Chemical Name | Chemical Structure |
|---|---|
| Ciglitazone | 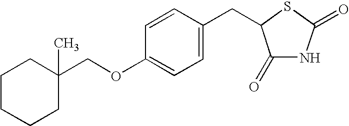 |
| Troglitazone | 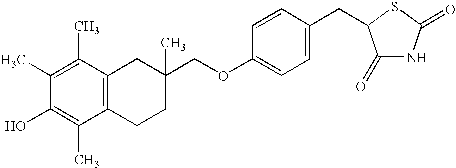 |
| Pioglitazone | 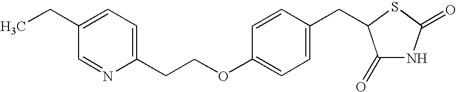 |
| Englitazone | 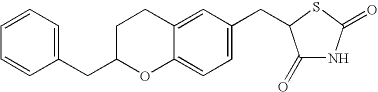 |
| Rosiglitazone | 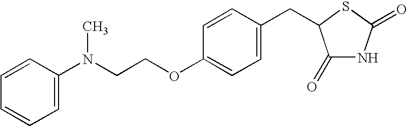 |
| Dargiltazone | 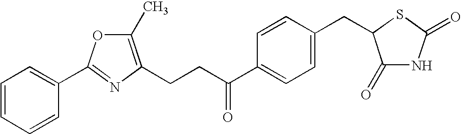 |
| KRP-297 | 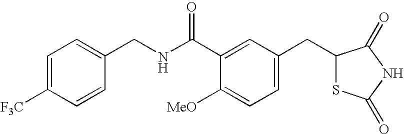 |
| MCC-555 (Isaglitazone) | 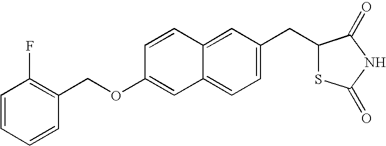 |
| JTT-501 | 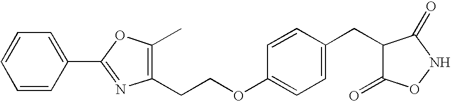 |
| DRF-2189 | 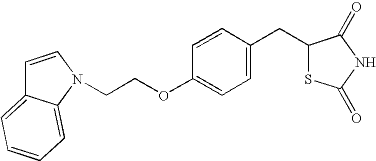 |

Yet another aspect of the invention comprises PPAR agonists that are members of the fibrate class of compounds. Examples of suitable fibrate compounds are shown in Table 5.

TABLE 5

| Chemical Name | Chemical Structure |
|---|---|
| Clofibrate | 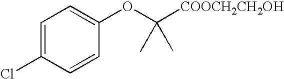 |
| Fenofibrate | 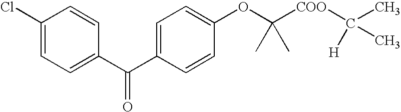 |
| Bezafibrate | 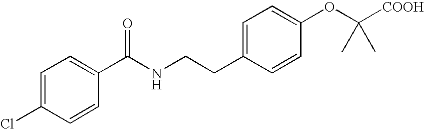 |

A further aspect of the invention comprises PPAR agonists that are members of the phenyl acetic acid class of compounds. Examples of suitable phenyl acetic acid compounds are shown in Table 6.

TABLE 6

| Chemical Name | Chemical Structure |
|---|---|
| SB-213068(RS) SB-236636(S) | 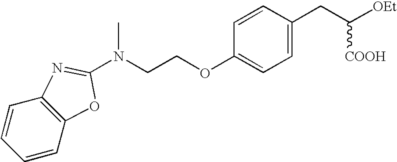 |
| SB-219994(S) | 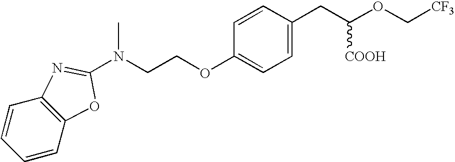 |
| GW-1929 | 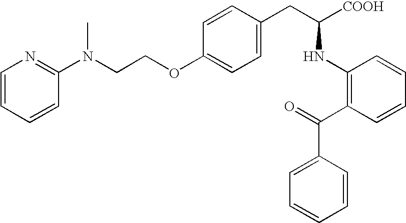 |
| GI-262570 | 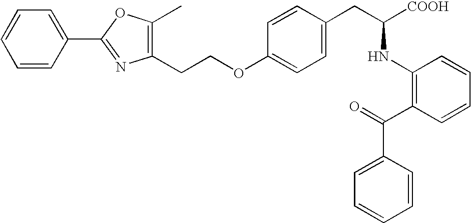 |
| GW-7845 | 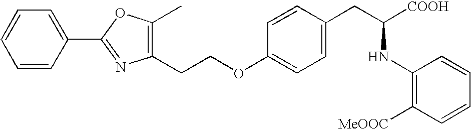 |
| L165041 | 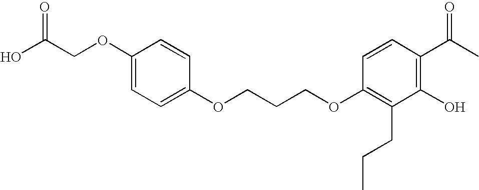 |
| GW 0207 | 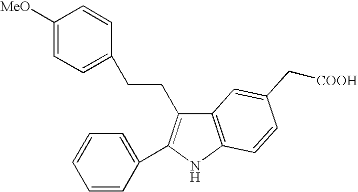 |
| X1 | 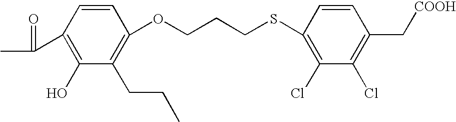 |
| X2 | 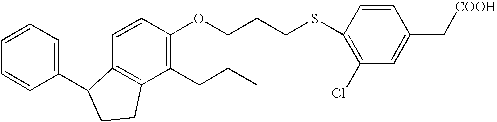 |

Yet a further aspect of the invention comprises the use of PPAR agonists detailed in Table 7.

TABLE 7

| Chemical | |
|---|---|
| Name | Chemical Structure |
| LG 100641 | 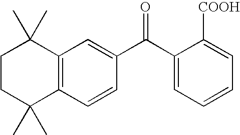 |
| L-764406 | 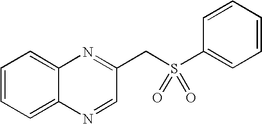 |
| GW 0072 | 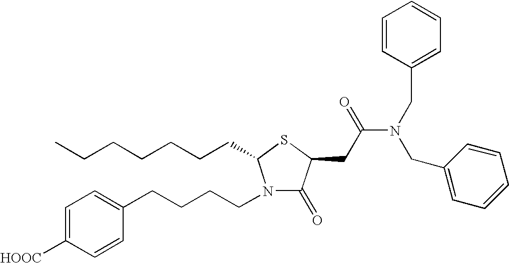 |

In yet another aspect of the invention, the PPAR agonist is a fatty acid analogue. Suitable fatty acid analogues that may be employed in the invention include prostaglandin A₂, prostaglandin J₂, tetradecylthioacetic acid and tetradecylselenoacetic acid.

Examples of additional PPAR agonists that may be employed in the current invention are detailed in U.S. Pat. Nos. 6,525,083 (B2) and 6,369,098 (B1), both of which are hereby incorporated by reference in their entirety.

Generally speaking, the pharmacokinetics of the particular agent to be administered will dictate the most preferred method of administration and dosing regiment. The PPAR agonist can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17^{th} Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Moreover, the PPAR agonist can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate.

In another embodiment, the PPAR agonist can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp. 197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The actual effective amounts of compound or drug can and will vary according to the specific composition being utilized, the mode of administration and the age, weight and condition of the subject. Dosages for a particular individual subject can be determined by one of ordinary skill in the art using conventional considerations.

By way of example, in one embodiment when the PPAR agonist is rosiglitazone, the amount administered daily is typically from about 0.5 to about 8 milligrams, and more typically, about 3 to about 7 milligrams.

By way of further example, in another embodiment when PPAR agonist is pioglitazone, the amount administered daily is typically from about 0.5 to about 45 milligrams, and more typically, about 15 to about 30 milligrams.

By way of yet further example, in another embodiment when PPAR agonist is troglitazone, the amount administered is typically about 200 to about 600 milligrams per day.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Generally speaking, when the composition is administered to treat an ischemic mediated condition, the PPAR agonist and cyclooxygenase-2 selective inhibitor are administered to the subject as soon as possible after the reduction in blood flow to the central nervous system in order to reduce the extent of ischemic damage. Typically, the PPAR agonist and cyclooxygenase-2 selective inhibitor are administered within 10 days after the reduction of blood flow to the central nervous system and more typically, within 24 hours. In still another embodiment, the PPAR agonist and cyclooxygenase-2 selective inhibitor are administered from about 1 to about 12 hours after the reduction in blood flow to the central nervous system. In another embodiment, the PPAR agonist and cyclooxygenase-2 selective inhibitor are administered in less than about 6 hours after the reduction in blood flow to the central nervous system. In still another embodiment, the PPAR agonist and cyclooxygenase-2 selective inhibitor are administered in less than about 4 hours after the reduction in blood flow to the central nervous system. In yet a further embodiment, the PPAR agonist and cyclooxygenase-2 selective inhibitor are administered in less than about 2 hours after the reduction in blood flow to the central nervous system.

Moreover, the timing of the administration of the cyclooxygenase-2 selective inhibitor in relation to the administration of the PPAR agonist may also vary from subject to subject. In one embodiment, the cyclooxygenase-2 selective inhibitor and PPAR agonist may be administered substantially simultaneously, meaning that both agents may be administered to the subject at approximately the same time. For example, the cyclooxygenase-2 selective is administered during a continuous period beginning on the same day as the beginning of the PPAR agonist and extending to a period after the end of the PPAR agonist. Alternatively, the cyclooxygenase-2 selective inhibitor and PPAR agonist may be administered sequentially, meaning that they are administered at separate times during separate treatments. In one embodiment, for example, the cyclooxygenase-2 selective inhibitor is administered during a continuous period beginning prior to administration of the PPAR agonist and ending after administration of the PPAR agonist. Of course, it is also possible that the cyclooxygenase-2 selective inhibitor may be administered either more or less frequently than the PPAR agonist. Moreover, it will be apparent to those skilled in the art that it is possible, and perhaps desirable, to combine various times and methods of administration in the practice of the present invention.

Combination Therapies

Generally speaking, it is contemplated that the composition employed in the practice of the invention may include one or more of any of the cyclooxygenase-2 selective inhibitors detailed above in combination with one or more of any of the PPAR agonists detailed above. By way of a non-limiting example, Table 8a details a number of suitable combinations that are useful in the methods and compositions of the current invention. The combination may also include an isomer, a pharmaceutically acceptable salt, ester, or prodrug of any of the cyclooxygenase-2 selective inhibitors or PPAR agonists listed in Table 8a.

TABLE 8a

| Cyclooxygenase-2 Selective | |
|---|---|
| Inhibitor | PPAR Agonists |
| a compound having formula I | ciglitazone |
| a compound having formula I | troglitazone |
| a compound having formula I | pioglitazone |
| a compound having formula I | englitazone |
| a compound having formula I | rosiglitazone |
| a compound having formula I | darglitazone |
| a compound having formula I | clofibrate |
| a compound having formula I | fenofibrate |
| a compound having formula I | bezafibrate |
| a compound having formula II | ciglitazone |
| a compound having formula II | troglitazone |
| a compound having formula II | pioglitazone |
| a compound having formula II | englitazone |
| a compound having formula II | rosiglitazone |
| a compound having formula II | darglitazone |
| a compound having formula II | clofibrate |
| a compound having formula II | fenofibrate |
| a compound having formula II | bezafibrate |
| a compound having formula III | ciglitazone |
| a compound having formula III | troglitazone |
| a compound having formula III | pioglitazone |
| a compound having formula III | englitazone |
| a compound having formula III | rosiglitazone |
| a compound having formula III | darglitazone |
| a compound having formula III | clofibrate |
| a compound having formula III | fenofibrate |
| a compound having formula III | bezafibrate |
| a compound having formula IV | ciglitazone |
| a compound having formula IV | troglitazone |
| a compound having formula IV | pioglitazone |
| a compound having formula IV | englitazone |
| a compound having formula IV | rosiglitazone |
| a compound having formula IV | darglitazone |
| a compound having formula IV | clofibrate |
| a compound having formula IV | fenofibrate |
| a compound having formula IV | bezafibrate |
| a compound having formula V | ciglitazone |
| a compound having formula V | troglitazone |
| a compound having formula V | pioglitazone |
| a compound having formula V | englitazone |
| a compound having formula V | rosiglitazone |
| a compound having formula V | darglitazone |
| a compound having formula V | clofibrate |
| a compound having formula V | fenofibrate |
| a compound having formula V | bezafibrate |

By way of further example, Table 8b details a number of suitable combinations that may be employed in the methods and compositions of the present invention. The combination may also include an isomer, a pharmaceutically acceptable salt, ester, or prodrug of any of the cyclooxygenase-2 selective inhibitors or PPAR agonists listed in Table 8b.

TABLE 8b

| Cyclooxygenase-2 Selective Inhibitor | PPAR Agonists |
|---|---|
| a compound selected from the group consisting | ciglitazone |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | troglitazone |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | pioglitazone |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | englitazone |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | rosiglitazone |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100,B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | darglitazone |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | clofibrate |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | fenofibrate |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |
| a compound selected from the group consisting | bezafibrate |
| of B-1, B-2, B-3, B-4, B-5, B-6, B-7, B-8, B-9, | |
| B-10, B-11, B-12, B-13, B-14, B-15, B-16, B-17, | |
| B-18, B-19, B-20, B-21, B-22, B-23, B-24, B-25, | |
| B-26, B-27, B-28, B-29, B-30, B-31, B-32, | |
| B-33, B-34, B-35, B-36, B-37, B-38, B-39, B-40, | |
| B-41, B-42, B-43, B-44, B-45, B-46, B-47, B-48, | |
| B-49, B-50, B-51, B-52, B-53, B-54, B-55, B-56, | |
| B-57, B-58, B-59, B-60, B-61, B-62, B-63, B-64, | |
| B-65, B-66, B-67, B-68, B-69, B-70, B-71, B-72, | |
| B-73, B-74, B-75, B-76, B-77, B-78, B-79, B-80, | |
| B-81, B-82, B-83, B-84, B-85, B-86, B-87, B-88, | |
| B-89, B-90, B-91, B-92, B-93, B-94, B-95, B-96, | |
| B-97, B-98, B-99, B-100, B-101, B-102, B-103, | |
| B-104, B-105, B-106, B-107, B-108, B-109, | |
| B-110, B-111, B-112, B-113, B-114, B-115, | |
| B-116, B-117, B-118, B-119, B-120, B-121, | |
| B-122, B-123, B-124, B-125, B-126, B-127, | |
| B-128, B-129, B-130, B-131, B-132, B-133, | |
| B-134, B-135, B-136, B-137, B-138, B-139, | |
| B-140, B-141, B-142, B-143, B-144, B-145, | |
| B-146, B-147, B-148, B-149, B-150, B-151, | |
| B-152, B-153, B-154, B-155, B-156, B-157, | |
| B-158, B-159, B-160, B-161, B-162, B-163, | |
| B-164, B-165, B-166, B-167, B-168, B-169, | |
| B-170, B-171, B-172, B-173, B-174, B-175, | |
| B-176, B-177, B-178, B-179, B-180, B-181, | |
| B-182, B-183, B-184, B-185, B-186, B-187, | |
| B-188, B-189, B-190, B-191, B-192, B-193, | |
| B-194, B-195, B-196, B-197, B-198, B-199, | |
| B-200, B-201, B-202, B-203, B-204, B-205, | |
| B-206, B-207, B-208, B-209, B-210, B-211, | |
| B-212, B-213, B-214, B-215, B-216, B-217, | |
| B-218, B-219, B-220, B-221, B-222, B-223, | |
| B-224, B-225, B-226, B-227, B-228, B-229, | |
| B-230, B-231, B-232, B233, B-234, B-235, B-236, | |
| B-237, B-238, B-239, B-240, B-241, B-242, B-243 | |
| B-244, B-245, B-246, B-247, B-248, B-249, | |
| B-250, B-251, B-252 | |

By way of yet further example, Table 8c details additional suitable combinations that may be employed in the methods and compositions of the current invention. The combination may also include an isomer, a pharmaceutically acceptable salt, ester, or prodrug of any of the cyclooxygenase-2 selective inhibitors or PPAR agonists listed in Table 8c.

TABLE 8c

| Cyclooxygenase-2 Selective Inhibitor | PPAR Agonists |
|---|---|
| Celecoxib | ciglitazone |
| Celecoxib | troglitazone |
| Celecoxib | pioglitazone |
| Celecoxib | englitazone |
| Celecoxib | rosiglitazone |
| Celecoxib | darglitazone |
| Celecoxib | clofibrate |
| Celecoxib | fenofibrate |
| Celecoxib | bezafibrate |
| Deracoxib | ciglitazone |
| Deracoxib | troglitazone |
| Deracoxib | pioglitazone |
| Deracoxib | englitazone |
| Deracoxib | rosiglitazone |
| Deracoxib | darglitazone |
| Deracoxib | clofibrate |
| Deracoxib | fenofibrate |
| Deracoxib | bezafibrate |
| Valdecoxib | ciglitazone |
| Valdecoxib | troglitazone |
| Valdecoxib | pioglitazone |
| Valdecoxib | englitazone |
| Valdecoxib | rosiglitazone |
| Valdecoxib | darglitazone |
| Valdecoxib | clofibrate |
| Valdecoxib | fenofibrate |
| Valdecoxib | bezafibrate |
| Rofecoxib | ciglitazone |
| Rofecoxib | troglitazone |
| Rofecoxib | pioglitazone |
| Rofecoxib | englitazone |
| Rofecoxib | rosiglitazone |
| Rofecoxib | darglitazone |
| Rofecoxib | clofibrate |
| Rofecoxib | fenofibrate |
| Rofecoxib | bezafibrate |
| Etoricoxib | ciglitazone |
| Etoricoxib | troglitazone |
| Etoricoxib | pioglitazone |
| Etoricoxib | englitazone |
| Etoricoxib | rosiglitazone |
| Etoricoxib | darglitazone |
| Etoricoxib | clofibrate |
| Etoricoxib | fenofibrate |
| Etoricoxib | bezafibrate |
| meloxicam | ciglitazone |
| meloxicam | troglitazone |
| meloxicam | pioglitazone |
| meloxicam | englitazone |
| meloxicam | rosiglitazone |
| meloxicam | darglitazone |
| meloxicam | clofibrate |
| meloxicam | fenofibrate |
| meloxicam | bezafibrate |
| Parecoxib | ciglitazone |
| Parecoxib | troglitazone |
| Parecoxib | pioglitazone |
| Parecoxib | englitazone |
| Parecoxib | rosiglitazone |
| Parecoxib | darglitazone |
| Parecoxib | clofibrate |
| Parecoxib | fenofibrate |
| Parecoxib | bezafibrate |
| 4-(4-cyclohexyl-2-methyloxazol-5-yl)-2- | ciglitazone |
| fluorobenzenesulfonamide | |
| 4-(4-cyclohexyl-2-methyloxazol-5-yl)-2- | troglitazone |
| fluorobenzenesulfonamide | |
| 4-(4-cyclohexyl-2-methyloxazol-5-yl)-2- | pioglitazone |
| fluorobenzenesulfonamide | |
| 4-(4-cyclohexyl-2-methyloxazol-5-yl)-2- | englitazone |
| fluorobenzenesulfonamide | |
| 4-(4-cyclohexyl-2-methyloxazol-5-yl)-2- | rosiglitazone |
| fluorobenzenesulfonamide | |
| 4-(4-cyclohexyl-2-methyloxazol-5-yl)-2- | darglitazone |
| fluorobenzenesulfonamide | |
| 4-(4-cyclohexyl-2-methyloxazol-5-yl)-2- | clofibrate |
| fluorobenzenesulfonamide | |
| 4-(4-cyclohexyl-2-methyloxazol-5-yl)-2- | fenofibrate |
| fluorobenzenesulfonamide | |
| 4-(4-cyclohexyl-2-methyloxazol-5-yl)-2- | bezafibrate |
| fluorobenzenesulfonamide | |
| 2-(3,5-difluorophenyl)-3-(4- | ciglitazone |
| (methylsulfonyl)phenyl)-2-cyclopenten-1-one | |
| 2-(3,5-difluorophenyl)-3-(4- | troglitazone |
| (methylsulfonyl)phenyl)-2-cyclopenten-1-one | |
| 2-(3,5-difluorophenyl)-3-(4- | pioglitazone |
| (methylsulfonyl)phenyl)-2-cyclopenten-1-one | |
| 2-(3,5-difluorophenyl)-3-(4- | englitazone |
| (methylsulfonyl)phenyl)-2-cyclopenten-1-one | |
| 2-(3,5-difluorophenyl)-3-(4- | rosiglitazone |
| (methylsulfonyl)phenyl)-2-cyclopenten-1-one | |
| 2-(3,5-difluorophenyl)-3-(4- | darglitazone |
| (methylsulfonyl)phenyl)-2-cyclopenten-1-one | |
| 2-(3,5-difluorophenyl)-3-(4- | clofibrate |
| (methylsulfonyl)phenyl)-2-cyclopenten-1-one | |
| 2-(3,5-difluorophenyl)-3-(4- | fenofibrate |
| (methylsulfonyl)phenyl)-2-cyclopenten-1-one | |
| 2-(3,5-difluorophenyl)-3-(4- | bezafibrate |
| (methylsulfonyl)phenyl)-2-cyclopenten-1-one | |
| N-[2-(cyclohexyloxy)-4- | ciglitazone |
| nitrophenyl]methanesulfonamide | |
| N-[2-(cyclohexyloxy)-4- | troglitazone |
| nitrophenyl]methanesulfonamide | |
| N-[2-(cyclohexyloxy)-4- | pioglitazone |
| nitrophenyl]methanesulfonamide | |
| N-[2-(cyclohexyloxy)-4- | englitazone |
| nitrophenyl]methanesulfonamide | |
| N-[2-(cyclohexyloxy)-4- | rosiglitazone |
| nitrophenyl]methanesulfonamide | |
| N-[2-(cyclohexyloxy)-4- | darglitazone |
| nitrophenyl]methanesulfonamide | |
| N-[2-(cyclohexyloxy)-4- | clofibrate |
| nitrophenyl]methanesulfonamide | |
| N-[2-(cyclohexyloxy)-4- | fenofibrate |
| nitrophenyl]methanesulfonamide | |
| N-[2-(cyclohexyloxy)-4- | bezafibrate |
| nitrophenyl]methanesulfonamide | |
| 2-(3,4-difluorophenyl)-4-(3-hydroxy-3- | ciglitazone |
| methylbutoxy)-5-[4- | |
| (methylsulfonyl)phenyl]-3(2H)-pyridazinone | |
| 2-(3,4-difluorophenyl)-4-(3-hydroxy-3- | troglitazone |
| methylbutoxy)-5-[4- | |
| (methylsulfonyl)phenyl]-3(2H)-pyridazinone | |
| 2-(3,4-difluorophenyl)-4-(3-hydroxy-3- | pioglitazone |
| methylbutoxy)-5-[4- | |
| (methylsulfonyl)phenyl]-3(2H)-pyridazinone | |
| 2-(3,4-difluorophenyl)-4-(3-hydroxy-3- | englitazone |
| methylbutoxy)-5-[4- | |
| (methylsulfonyl)phenyl]-3(2H)-pyridazinone | |
| 2-(3,4-difluorophenyl)-4-(3-hydroxy-3- | rosiglitazone |
| methylbutoxy)-5-[4- | |
| (methylsulfonyl)phenyl]-3(2H)-pyridazinone | |
| 2-(3,4-difluorophenyl)-4-(3-hydroxy-3- | darglitazone |
| methylbutoxy)-5-[4- | |
| (methylsulfonyl)phenyl]-3(2H)-pyridazinone | |
| 2-(3,4-difluorophenyl)-4-(3-hydroxy-3- | clofibrate |
| methylbutoxy)-5-[4- | |
| (methylsulfonyl)phenyl]-3(2H)-pyridazinone | |
| 2-(3,4-difluorophenyl)-4-(3-hydroxy-3- | fenofibrate |
| methylbutoxy)-5-[4- | |
| (methylsulfonyl)phenyl]-3(2H)-pyridazinone | |
| 2-(3,4-difluorophenyl)-4-(3-hydroxy-3- | bezafibrate |
| methylbutoxy)-5-[4- | |
| (methylsulfonyl)phenyl]-3(2H)-pyridazinone | |
| 2-[(2,4-dichloro-6-methylphenyl)amino]-5- | ciglitazone |
| ethyl-benzeneacetic acid | |
| 2-[(2,4-dichloro-6-methylphenyl)amino]-5- | troglitazone |
| ethyl-benzeneacetic acid | |
| 2-[(2,4-dichloro-6-methylphenyl)amino]-5- | pioglitazone |
| ethyl-benzeneacetic acid | |
| 2-[(2,4-dichloro-6-methylphenyl)amino]-5- | englitazone |
| ethyl-benzeneacetic acid | |
| 2-[(2,4-dichloro-6-methylphenyl)amino]-5- | rosiglitazone |
| ethyl-benzeneacetic acid | |
| 2-[(2,4-dichloro-6-methylphenyl)amino]-5- | darglitazone |
| ethyl-benzeneacetic acid | |
| 2-[(2,4-dichloro-6-methylphenyl)amino]-5- | clofibrate |
| ethyl-benzeneacetic acid | |
| 2-[(2,4-dichloro-6-methylphenyl)amino]-5- | fenofibrate |
| ethyl-benzeneacetic acid | |
| 2-[(2,4-dichloro-6-methylphenyl)amino]-5- | bezafibrate |
| ethyl-benzeneacetic acid | |
| (3Z)-3-[(4-chlorophenyl)[4- | ciglitazone |
| (methylsulfonyl)phenyl]methylene]dihydro- | |
| 2(3H)-furanone | |
| (3Z)-3-[(4-chlorophenyl)[4- | troglitazone |
| (methylsulfonyl)phenyl]methylene]dihydro- | |
| 2(3H)-furanone | |
| (3Z)-3-[(4-chlorophenyl)[4- | pioglitazone |
| (methylsulfonyl)phenyl]methylene]dihydro- | |
| 2(3H)-furanone | |
| (3Z)-3-[(4-chlorophenyl)[4- | englitazone |
| (methylsulfonyl)phenyl]methylene]dihydro- | |
| 2(3H)-furanone | |
| (3Z)-3-[(4-chlorophenyl)[4- | rosiglitazone |
| (methylsulfonyl)phenyl]methylene]dihydro- | |
| 2(3H)-furanone | |
| (3Z)-3-[(4-chlorophenyl)[4- | darglitazone |
| (methylsulfonyl)phenyl]methylene]dihydro- | |
| 2(3H)-furanone | |
| (3Z)-3-[(4-chlorophenyl)[4- | clofibrate |
| (methylsulfonyl)phenyl]methylene]dihydro- | |
| 2(3H)-furanone | |
| (3Z)-3-[(4-chlorophenyl)[4- | fenofibrate |
| (methylsulfonyl)phenyl]methylene]dihydro- | |
| 2(3H)-furanone | |
| (3Z)-3-[(4-chlorophenyl)[4- | bezafibrate |
| (methylsulfonyl)phenyl]methylene]dihydro- | |
| 2(3H)-furanone | |
| (S)-6,8-dichloro-2-(trifluoromethyl)-2H-1- | ciglitazone |
| benzopyran-3-carboxylic acid | |
| (S)-6,8-dichloro-2-(trifluoromethyl)-2H-1- | troglitazone |
| benzopyran-3-carboxylic acid | |
| (S)-6,8-dichloro-2-(trifluoromethyl)-2H-1- | pioglitazone |
| benzopyran-3-carboxylic acid | |
| (S)-6,8-dichloro-2-(trifluoromethyl)-2H-1- | englitazone |
| benzopyran-3-carboxylic acid | |
| (S)-6,8-dichloro-2-(trifluoromethyl)-2H-1- | rosiglitazone |
| benzopyran-3-carboxylic acid | |
| (S)-6,8-dichloro-2-(trifluoromethyl)-2H-1- | darglitazone |
| benzopyran-3-carboxylic acid | |
| (S)-6,8-dichloro-2-(trifluoromethyl)-2H-1- | clofibrate |
| benzopyran-3-carboxylic acid | |
| (S)-6,8-dichloro-2-(trifluoromethyl)-2H-1- | fenofibrate |
| benzopyran-3-carboxylic acid | |
| (S)-6,8-dichloro-2-(trifluoromethyl)-2H-1- | bezafibrate |
| benzopyran-3-carboxylic acid | |
| lumiracoxib | ciglitazone |
| lumiracoxib | troglitazone |
| lumiracoxib | pioglitazone |
| lumiracoxib | englitazone |
| lumiracoxib | rosiglitazone |
| lumiracoxib | darglitazone |
| lumiracoxib | clofibrate |
| lumiracoxib | fenofibrate |
| lumiracoxib | bezafibrate |

Diagnosis of a Vaso-Occlusion

One aspect of the invention encompasses diagnosing a subject in need of treatment or prevention for a vaso-occlusive event. A number of suitable methods for diagnosing a vaso-occlusion may be used in the practice of the invention. In one such method, ultrasound may be employed. This method examines the blood flow in the major arteries and veins in the arms and legs with the use of ultrasound (high-frequency sound waves). In one embodiment, the test may combine Doppler® ultrasonography, which uses audio measurements to “hear” and measure the blood flow and duplex ultrasonography, which provides a visual image. In an alternative embodiment, the test may utilize multifrequency ultrasound or multifrequency transcranial Doppler® (MTCD) ultrasound.

Another method that may be employed encompasses injection of the subject with a compound that can be imaged. In one alternative of this embodiment, a small amount of radioactive material is injected into the subject and then standard techniques that rely on monitoring blood flow to detect a blockage, such as magnetic resonance direct thrombus imaging (MRDTI), may be utilized to image the vaso-occlusion. In an alternative embodiment, ThromboView® (commercially available from Agenix Limited) uses a clot-binding monoclonal antibody attached to a radiolabel. In addition to the methods identified herein, a number of other suitable methods known in the art for diagnosis of vaso-occlusive events may be utilized.

Indications to be Treated

Typically, the composition comprising a therapeutically effective amount of a cyclooxygenase-2 selective inhibitor and a therapeutically effective amount of a PPAR agonist may be employed to treat a number of ischemic mediated central nervous system disorders.

In some aspects, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. By way of example, the normal amount of perfusion to brain gray matter in humans is about 60 to 70 mL/100 g of brain tissue/min. Death of central nervous system cells typically occurs when the flow of blood falls below approximately 8-10 mL/100 g of brain tissue/min, while at slightly higher levels (i.e. 20-35 mL/100 g of brain tissue/min) the tissue remains alive but not able to function. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell.

Another aspect encompasses administrating the composition to a subject to treat a central nervous system ischemic condition. A number of central nervous system ischemic conditions may be treated by the composition of the invention. In one embodiment, the ischemic condition is a stroke that results in any type of ischemic central nervous system damage, such as apoptotic or necrotic cell death, cytoxic edema or central nervous system tissue anoxemia. The stroke may impact any area of the brain or be caused by any etiology commonly known to result in the occurrence of a stroke. In one alternative of this embodiment, the stroke is a brain stem stroke. Generally speaking, brain stem strokes strike the brain stem, which control involuntary life-support functions such as breathing, blood pressure, and heartbeat. In another alternative of this embodiment, the stroke is a cerebellar stroke. Typically, cerebellar strokes impact the cerebellum area of the brain, which controls balance and coordination. In still another embodiment, the stroke is an embolic stroke. In general terms, embolic strokes may impact any region of the brain and typically result from the blockage of an artery by a vaso-occlusion. In yet another alternative, the stroke may be a hemorrhagic stroke. Like embolic strokes, hemorrhagic stroke may impact any region of the brain, and typically result from a ruptured blood vessel characterized by a hemorrhage (bleeding) within or surrounding the brain. In a further embodiment, the stroke is a thrombotic stroke. Typically, thrombotic strokes result from the blockage of a blood vessel by accumulated deposits.

In another embodiment, the ischemic condition may result from a disorder that occurs in a part of the subject's body outside of the central nervous system, but yet still causes a reduction in blood flow to the central nervous system. These disorders may include, but are not limited to a peripheral vascular disorder, a venous thrombosis, a pulmonary embolus, a myocardial infarction, a transient ischemic attack, unstable angina, or sickle cell anemia. Moreover, the central nervous system ischemic condition may occur as result of the subject undergoing a surgical procedure. By way of example, the subject may be undergoing heart surgery, lung surgery, spinal surgery, brain surgery, vascular surgery, abdominal surgery, or organ transplantation surgery. The organ transplantation surgery may include heart, lung, pancreas or liver transplantation surgery. Moreover, the central nervous system ischemic condition may occur as a result of a trauma or injury to a part of the subject's body outside the central nervous system. By way of example the trauma or injury may cause a degree of bleeding that significantly reduces the total volume of blood in the subject's body. Because of this reduced total volume, the amount of blood flow to the central nervous system is concomitantly reduced. By way of further example, the trauma or injury may also result in the formation of a vaso-occlusion that restricts blood flow to the central nervous system.

Of course it is contemplated that the composition may be employed to treat the central nervous system ischemic condition irrespective of the cause of the condition. In one embodiment, the ischemic condition results from a vaso-occlusion. The vaso-occlusion may be any type of occlusion, but is typically a cerebral thrombosis or a cerebral embolism. In a further embodiment, the ischemic condition may result from a hemorrhage. The hemorrhage may be any type of hemorrhage, but is generally a cerebral hemorrhage or a subararachnoid hemorrhage. In still another embodiment, the ischemic condition may result from the narrowing of a vessel. Generally speaking, the vessel may narrow as a result of a vasoconstriction such as occurs during vasospasms, or due to arteriosclerosis. In yet another embodiment, the ischemic condition results from an injury to the brain or spinal cord.

In yet another aspect, the composition is administered to reduce infarct size of the ischemic core following a central nervous system ischemic condition. Moreover, the composition may also be beneficially administered to reduce the size of the ischemic penumbra or transitional zone following a central nervous system ischemic condition.

In a further aspect, the invention provides treatment for subjects who are at risk of a vaso-occlusive event. These subjects may or may not have had a previous vaso-occlusive event. The invention embraces the treatment of subjects prior to a vaso-occlusive event, at a time of a vaso-occlusive event and following a vaso-occlusive event. Thus, as used herein, the “treatment” of a subject is intended to embrace both prophylactic and therapeutic treatment, and can be used either to limit or to eliminate altogether the symptoms or the occurrence of a vaso-occlusive event.

In addition to a cyclooxygenase-2 selective inhibitor and a PPAR agonist, the composition of the invention may also include any agent that ameliorates the effect of a reduction in blood flow to the central nervous system. In one embodiment, the agent is an anticoagulant including thrombin inhibitors such as heparin and Factor Xa inhibitors such as warafin. In an additional embodiment, the agent is an anti-platelet inhibitor such as a GP IIb/IIIa inhibitor. Additional agents include but are not limited to, HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors; probucol; niacin; fibrates such as clofibrate, fenofibrate, and gemfibrizol; cholesterol absorption inhibitors; bile acid sequestrants; LDL (low density lipoprotein) receptor inducers; vitamin B₆ (also known as pyridoxine) and the pharmaceutically acceptable salts thereof such as the HCl salt; vitamin B₁₂ (also known as cyanocobalamin); β-adrenergic receptor blockers; folic acid or a pharmaceutically acceptable salt or ester thereof such as the sodium salt and the methylglucamine salt; and anti-oxidant vitamins such as vitamin C and E and beta carotene.

In a further aspect, the composition may be employed to reverse or lessen central nervous system cell damage following a traumatic brain or spinal cord injury. Traumatic brain or spinal cord injury may result from a wide variety of causes including, for example, blows to the head or back from objects; penetrating injuries from missiles, bullets, and shrapnel; falls; skull fractures with resulting penetration by bone pieces; and sudden acceleration or deceleration injuries. The composition of the invention may be beneficially utilized to treat the traumatic injury irrespective of its cause.

### EXAMPLES

A combination therapy of a COX-2 selective inhibitor and a PPAR agonist for the treatment or prevention of a vaso-occlusive event or a related disorder in a subject can be evaluated as described in the following tests detailed below.

A particular combination therapy comprising a PPAR agonist and a COX-2 inhibitor can be evaluated in comparison to a control treatment such as a placebo treatment, administration of a COX-2 inhibitor only, or administration of a PPAR agonist only. By way of example, a combination therapy may contain any of the PPAR agonists and COX-2 inhibitors detailed in the present invention, including the combinations set forth in Tables 8a, 8b, or 8c may be tested as a combination therapy. The dosages of a PPAR agonist and COX-2 inhibitor in a particular therapeutic combination may be readily determined by a skilled artisan conducting the study. The length of the study treatment will vary on a particular study and can also be determined by one of ordinary skill in the art. By way of example, the combination therapy may be administered for 4 weeks. The PPAR agonist and COX-2 inhibitor can be administered by any route as described herein, but are preferably administered orally for human subjects.

### Example 1

### Evaluation of COX-1 and COX-2 Activity In Vitro

The COX-2 inhibitors suitable for use in this invention exhibit selective inhibition of COX-2 over COX-1 when tested in vitro according to the following activity assays.

Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10⁷-10⁸ pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C. by transferring 40 μl of reaction mix into 160 μl ELISA buffer and 25 μM indomethacin. The PGE2 formed is measured by standard ELISA technology (Cayman Chemical).

Fast Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C. by transferring 40 μl of reaction mix into 160 μl ELISA buffer and 25 μM indomethacin. Indomethacin, a non-selective COX-2/COX-1 inhibitor, may be utilized as a positive control. The PGE₂ formed is typically measured by standard ELISA technology utilizing a PGE2 specific antibody, available from a number of commercial sources.

Each compound to be tested may be individually dissolved in 2 ml of dimethyl sulfoxide (DMSO) for bioassay testing to determine the COX-1 and COX-2 inhibitory effects of each particular compound. Potency is typically expressed by the IC₅₀ value expressed as g compound/ml solvent resulting in a 50% inhibition of PGE2 production. Selective inhibition of COX-2 may be determined by the IC₅₀ ratio of COX-1/COX-2.

By way of example, a primary screen may be performed in order to determine particular compounds that inhibit COX-2 at a concentration of 10 ug/ml. The compound may then be subjected to a confirmation assay to determine the extent of COX-2 inhibition at three different concentrations (e.g., 10 ug/ml, 3.3 ug/ml and 1.1 ug/ml). After this screen, compounds can then be tested for their ability to inhibit COX-1 at a concentration of 10 ug/ml. With this assay, the percentage of COX inhibition compared to control can be determined, with a higher percentage indicating a greater degree of COX inhibition. In addition, the IC₅₀ value for COX-1 and COX-2 can also be determined for the tested compound. The selectivity for each compound may then be determined by the IC₅₀ ratio of COX-1/COX-2, as set-forth above.

### Example 2

### Methods for Measuring Platelet Aggregation and Platelet Activation Markers

The following studies can be performed in human subjects or laboratory animal models, such as mice. Prior to the initiation of a clinical study involving human subjects, the study should be approved by the appropriate Human Subjects Committee and subjects should be informed about the study and give written consent prior to participation.

Platelet activation can be determined by a number of tests available in the art. Several such tests are described below. In order to determine the effectiveness of the treatment, the state of platelet activation is evaluated at several time points during the study, such as before administering the combination treatment and once a week during treatment. The exemplary procedures for blood sampling and the analyses that can be used to monitor platelet aggregation are listed below.

Platelet Aggregation Study

Blood samples are collected from an antecubital vein via a 19-gauge needle into two plastic tubes. Each sample of free flowing blood is collected through a fresh venipuncture site distal to any intravenous catheters using a needle and Vacutainer hood into 7 cc vacutainer tubes (one with CTAD (dipyridamole), and the other with 3.8% trisodium citrate). If blood is collected simultaneously for any other studies, it is preferable that the platelet sample be obtained second or third, but not first. If only the platelet sample is collected, the initial 2-3 cc of blood is discharged and then the vacutainer tube is filled. The venipuncture is adequate if the tube fills within 15 seconds. All collections are performed by trained personnel.

After the blood samples for each subject have been collected into two Vacutainer tubes, they are immediately, but gently, inverted 3 to 5 times to ensure complete mixing of the anticoagulant. Tubes are not shaken. The Vacutainer tubes are filled to capacity, since excess anticoagulant can alter platelet function. Attention is paid to minimizing turbulence whenever possible. Small steps, such as slanting the needle in the Vacutainer to have the blood run down the side of tube instead of shooting all the way to the bottom, can result in significant improvement. These tubes are kept at room temperature and transferred directly to the laboratory personnel responsible for preparing the samples. The Vacutainer tubes are not chilled at any time.

Trisodium citrate (3.8%) and whole blood is immediately mixed in a 1:9 ratio, and then centrifuged at 1200 g for 2.5 minutes, to obtain platelet-rich plasma (PRP), which is kept at room temperature for use within 1 hour for platelet aggregation studies. Platelet count is determined in each PRP sample with a Coulter Counter ZM (Coulter Co., Hialeah, Fla.). Platelet numbers are adjusted to 3.50×10⁸/ml for aggregation with homologous platelet-poor plasma. PRP and whole blood aggregation tests are performed simultaneously. Whole blood is diluted 1:1 with the 0.5 ml PBS, and then swirled gently to mix. The cuvette with the stirring bar is placed in the incubation well and allowed to warm to 37° C. for 5 minutes. Then the samples are transferred to the assay well. An electrode is placed in the sample cuvette. Platelet aggregation is stimulated with 5 μM ADP, 1 μg/ml collagen, and 0.75 mM arachidonic acid. All agonists are obtained, e.g., from Chronolog Corporation (Hawertown, Pa.). Platelet aggregation studies are performed using a Chrono-Log Whole Blood Lumi-Aggregometer (model 560-Ca). Platelet aggregability is expressed as the percentage of light transmittance change from baseline using platelet-poor plasma as a reference at the end of recording time for plasma samples, or as a change in electrical impedance for whole blood samples. Aggregation curves are recorded for 4 minutes and analyzed according to internationally established standards using Aggrolink® software.

Aggregation curves of subjects receiving a combination therapy containing a PPAR agonist and a COX-2 inhibitor can then be compared to the aggregation curves of subjects receiving a control treatment in order to determine the efficacy of said combination therapy.

Washed Platelets Flow Cytometry

Venous blood (8 ml) is collected in a plastic tube containing 2 ml of acid-citrate-dextrose (ACD) (7.3 g citric acid, 22.0 g sodium citrate×2H₂O and 24.5 glucose in 1000 ml distilled water) and mixed well. The blood-ACD mixture is centrifuged at 1000 r.p.m. for 10 minutes at room temperature. The upper ⅔ of the platelet-rich plasma (PRP) is then collected and adjusted to pH=6.5 by adding ACD. The PRP is then centrifuged at 3000 r.p.m. for 10 minutes. The supernatant is removed and the platelet pellet is gently resuspended in 4 cc of the washing buffer (10 mM Tris/HCl, 0.15 M NaCl, 20 mM EDTA, pH=7.4). Platelets are washed in the washing buffer, and in TBS (10 mM Tris, 0.15 M NaCl, pH=7.4). All cells are then divided into the appropriate number of tubes. By way of example, if 9 different surface markers are evaluated, as described herein, then the cells should be divided into ten tubes, such that nine tubes containing washed platelets are incubated with 5 μl fluorescein isothiocyanate (FITC)-conjugated antibodies in the dark at +4° C. for 30 minutes, and one tube remains unstained and serves as a negative control. Surface antigen expression is measured with monoclonal murine anti-human antibodies, such as CD9 (p24); CD41a (IIb/IIIa, aIIbb3); CD42b (Ib); CD61 (IIIa) (DAKO Corporation, Carpinteria, Calif.); CD49b (VLA-2, or a2b1); CD62p (P-selectin); CD31 (PECAM-1); CD 41b (IIb); and CD51/CD61 (vitronectin receptor, avb3) (PharMingen, San Diego Calif.), as the expression of these antigens on the cells is associated with platelet activation. After incubation, the cells are washed with TBS and resuspended in 0.25 ml of 1% paraformaldehyde. Samples are stored in the refrigerator at +4° C., and analyzed on a Becton Dickinson FACScan flow cytometer with laser output of 15 mw, excitation at 488 nm, and emission detection at 530+−30 nm. The data can be collected and stored in list mode, and then analyzed using CELLQuest® software. FACS procedures are described in detail in, e.g., Gurbel, P. A. et al., J Amer Coll Cardiol 31: 1466-1473 (1998); Serebruany, V. L. et al., Am Heart J 136: 398-405 (1998); Gurbel, P. A. et al., Coron Artery Dis 9: 451-456 (1998) and Serebruany, V. L. et al., Arterioscl Thromb Vasc Biol 19: 153-158 (1999).

The antibody staining of platelets isolated from subjects receiving a combination therapy can then be compared to the staining of platelets isolated from subjects receiving a control treatment in order to determine the effect of the combination therapy on platelets.

Whole Blood Flow Cytometry

Four cc of blood is collected in a tube, containing 2 cc of acid-citrate-dextrose (ACD, see previous example) and mixed well. The buffer, TBS (10 mM Tris, 0.15 M NaCl, pH 7.4) and the following fluorescein isothiocyanate (FITC) conjugated monoclonal antibodies (PharMingen, San Diego, Calif., USA, and DAKO, Calif., USA) are removed from a refrigerator and allowed to warm at room temperature (RT) prior to their use. The non-limiting examples of antibodies that can be used include CD41 (IIb/IIIa), CD31 (PECAM-1), CD62p (P-selectin), and CD51/61 (Vitronectin receptor). For each subject, six amber tubes (1.25 ml) are one Eppendorf tube (1.5 ml) are obtained and marked appropriately. 450 μl of TBS buffer is pipetted to the labeled Eppendorf tube. A patient's whole blood tube is inverted gently twice to mix, and 50 μl of whole blood is pipetted to the appropriately labeled Eppendorf tube. The Eppendorf tube is capped and the diluted whole blood is mixed by inverting the Eppendorf tube gently two times, followed by pipetting 501 μl of diluted whole blood to each amber tube. 5 μl of appropriate antibody is pipetted to the bottom of the corresponding amber tube. The tubes are covered with aluminum foil and incubated at 4° C. for 30 minutes. After incubation, 400 μl of 2% buffered paraformaldehyde is added. The amber tubes are closed with a lid tightly and stored in a refrigerator at 4° C. until the flow cytometric analysis. The samples are analyzed on a Becton Dickinson FACScan flow cytometer. These data are collected in list mode files and then analyzed. As mentioned in (B.), the antibody staining of platelets isolated from subjects receiving a combination therapy can then be compared to the staining of platelets isolated from subjects receiving a control treatment.

ELISA

Enzyme-linked immunosorbent assays (ELISA) are used according to standard techniques and as described herein. Eicosanoid metabolites may be used to determine platelet aggregation. The metabolites are analyzed due to the fact that eicosanoids have a short half-life under physiological conditions. Thromboxane B2 (TXB₂), the stable breakdown product of thromboxane A₂ and 6keto-PGF₁ alpha, the stable degradation product of prostacyclin may be tested. Thromboxane B2 is a stable hydrolysis product of TXA₂ and is produced following platelet aggregation induced by a variety of agents, such as thrombin and collagen. 6keto-prostaglandin F₁ alpha is a stable hydrolyzed product of unstable PGI₂ (prostacyclin). Prostacyclin inhibits platelet aggregation and induces vasodilation. Thus, quantitation of prostacyclin production can be made by determining the level of 6keto-PGF₁. The metabolites may be measured in the platelet poor plasma (PPP), which is kept at −4° C. Also, plasma samples may also be extracted with ethanol and then stored at −80° C. before final prostaglandin determination, using, e.g., TiterZymes® enzyme immunoassays according to standard techniques (PerSeptive Diagnostics, Inc., Cambridge, Mass., USA). ELISA kits for measuring TXB₂ and 6keto-PGF₁ are also commercially available.

The amounts of TXB₂ and 6keto-PGF₁ in plasma of subjects receiving a combination therapy and subjects receiving a control therapy can be compared to determine the efficacy of the combination treatment.

Closure Time Measured with the Dade Behring Platelet Function Analyzer, PFA-100®

PFA-100® can be used as an in vitro system for the detection of platelet dysfunction. It provides a quantitative measure of platelet function in anticoagulated whole blood. The system comprises a microprocessor-controlled instrument and a disposable test cartridge containing a biologically active membrane. The instrument aspirates a blood sample under constant vacuum from the sample reservoir through a capillary and a microscopic aperture cut into the membrane. The membrane is coated with collagen and epinephrine or adenosine 5′-diphosphate. The presence of these biochemical stimuli, and the high shear rates generated under the standardized flow conditions, result in platelet attachment, activation, and aggregation, slowly building a stable platelet plug at the aperture. The time required to obtain full occlusion of the aperture is reported as the “closure time,” which normally ranges from one to three minutes.

The membrane in the PFA-100® test cartridge serves as a support matrix for the biological components and allows placement of the aperture. The membrane is a standard nitrocellulose filtration membrane with an average pore size of 0.45 μm. The blood entry side of the membrane was coated with 2 μg of fibrillar Type I equine tendon collagen and 10 μg of epinephrine bitartrate or 50 μg of adenosine 5′-diphosphate (ADP). These agents provide controlled stimulation to the platelets as the blood sample passes through the aperture. The collagen surface also served as a well-defined matrix for platelet deposition and attachment.

The principle of the PFA-100® testis very similar to that described by Kratzer and Born (Kratzer, et al., Haemostasis 15: 357-362 (1985)). The test utilizes whole blood samples collected in 3.8% of 3.2% sodium citrate anticoagulant. The blood sample is aspirated through the capillary into the cup where it comes in contact with the coated membrane, and then passes through the aperture. In response to the stimulation by collagen and epinephrine or ADP present in the coating, and the shear stresses at the aperture, platelets adhere and aggregate on the collagen surface starting at the area surrounding the aperture. During the course of the measurement, a stable platelet plug forms that ultimately occludes the aperture. The time required to obtain full occlusion of the aperture is defined as the “closure time” and is indicative of the platelet function in the sample. Accordingly, “closure times” can be compared between subjects receiving a combination therapy and the ones receiving a control therapy in order to evaluate the efficacy of the combination treatment.

### Example 3

The laboratory animal study can generally be performed as described in Tanaka et al., Neurochemical Research, Vol. 20, No. 6, 1995, pp. 663-667.

Briefly, the study can be performed with about 30 gerbils, with body weights of 65 to 80 grams. The animals are anesthetized with ketamine (100 mg/kg body weight, i.p.), and silk threads are placed around both common carotid arteries without interrupting carotid artery blood flow. On the next day, bilateral common carotid arteries are exposed and then occluded with surgical clips after light ether anesthesia (see, e.g., Ogawa et al., Adv. Exp. Med. Biol., 287:343-347, and Ogawa et al., Brain Res., 591:171-175). Carotid artery blood flow is restored by releasing the clips after 5 minutes of occlusion. Body temperature is maintained about 37° C. using a heating pad and an incandescent lamp. Control animals are operated on in a similar manner but the carotid arteries are not occluded. The combination therapy is administered immediately and 6 and 12 hours after recirculation in the ischemia group, whereas sham-operated animals receive placebo, which may be, e.g., the vehicle used to administer the combination therapy. Gerbils are sacrificed by decapitation 14 days after recirculation. The brain is removed rapidly and placed on crushed dry-ice to freeze the tissue.

The brain tissue can then be examined histologically for the effects of combination therapy in comparison to the placebo. For example, each brain is cut into 14 μm thick sections at −15° C. Coronal sections that include the cerebral cortex and hippocampal formation are thawed, mounted onto gelatin-coated slides, dried completely, and fixed with 10% formalin for 2 hours. The sections are stained with hematoxylin-eosin and antibodies to glial fibrillary acidic protein (GFAP), which can be commercially obtained from, e.g., Nichirei, Tokyo, Japan. Immune complexes are detected by the avidin-biotin interaction and visualized with 3,3′-diaminobenzidine tetrahydrochloride. Sections that are used as controls are stained in a similar manner without adding anti-GFAP antibody. The densities of living pyramidal cells and GFAP-positive astrocytes in the typical CA1 subfield of the hippocampus are calculated by counting the cells and measuring the total length of the CA1 cell layer in each section from 250× photomicrographs. The average densities of pyramidal cells and GFAP-positive astrocytes in the CA1 subfield for each gerbil are obtained from counting cells in one unit area in each of these sections of both left and right hemispheres.

The effects of the combination therapy in comparison with the placebo can be determined both qualitatively and quantitatively. For example, the appearance of CA1 pyramidal neurons and pyramidal cell density in the CA1 subfield may be used to assess the efficacy of the treatment. In addition, immunohistological analysis can reveal the efficacy of combination by evaluating the presence or absence of hypertrophic GFAP-positive astrocytes in the CA1 region of treated gerbils, since the sham-operated animals should have few GFAP-positive astrocytes.

### Example 4

Rat middle cerebral artery occlusion (MCAO) models are well known in the art and useful in assessing a neuroprotective drug efficacy in stroke. By way of example, the methods and materials for MCAO model described in Turski et al. (Proc. Natl. Acad, Sci. USA, Vol. 95, pp. 10960-10965, September 1998) may be modified for testing the combination therapy as described above for cerebral ischemia treatment.

The permanent middle cerebral artery occlusion can be established by means of microbipolar permanent coagulation in, e.g., Fisher 344 rats (260-290 grams) anesthetized with halothane as described previously in, e.g., Lippert et al., Eur. J. Pharmacol., 253, pp. 207-213, 1994. To determine the efficacy of the combination treatment and the therapeutic window for such treatment, the combination therapy can be administered, e.g., intravenously over 6 hours beginning 1, 2, 4, 5, 6, 7, 12, or 24 hours after MCAO. It should be noted that different doses, routes of administrations, and times of administration can also be readily tested. Furthermore, the experiment should be controlled appropriately, e.g. by administering placebo to a set of MCAO-induced rats. To evaluate the efficacy of the combination therapy, the size of infarct in the brain can be estimated stereologically, e.g., seven days after MCAO, by means of advanced image analysis.

In addition, the assessment of neuroprotective action against focal cerebral reperfusion ischemia can be performed in Wistar rats (250-300 grams) that are anesthetized with halothane and subjected to temporary occlusion of the common carotid arteries and the right middle cerebral artery (CCA/MCAO) for 90 minutes. CCAs can be occluded by means of silastic threads placed around the vessels, and MCA can be occluded by means of a steel hook attached to a micromanipulator. Blood flow stop can be verified by microscopic examination of the MCA or laser doppler flowmetry. Different doses of combination therapy can then be administered over, e.g., 6 hours starting immediately after the beginning of reperfusion or, e.g., 2 hours after the onset of reperfusion. As mentioned previously, the size of infarct in the brain can be estimated, for example, stereologically seven days after CCA/MCAO by means of image analysis.

It should be noted that all of the above-mentioned procedures can be modified for a particular study, depending on factors such as a drug combination used, length of the study, subjects that are selected, etc. Such modifications can be designed by a skilled artisan without undue experimentation.

### Example 5

### Focal Cerebral Ischemia Study

The following procedures can be performed as described in, e.g., Nogawa et al., Journal of Neuroscience, 17(8):2746-2755, Apr. 15, 1997.

The middle cerebral artery (MCA) is transiently occluded in a number of Sprague Dawley rats, weighing 275-310 grams, using an intravascular occlusion model, as described in, e.g., Longa et al., Stroke 20:84-91, 1989, Iadecola et al., Stroke 27:1373-1380, 1996, and Zhang et al., Stroke 27:317-323. A skilled artisan can readily determine the appropriate number of animals to be used for a particular experiment. Under halothane anesthesia (induction 5%, maintenance 1%), a 4-0 nylon monofilament with a rounded tip is inserted centripetally into the external carotid artery and advanced into the internal carotid artery until it reaches the circle of Willis. Throughout the procedure, body temperature is maintained at 37°±0.5° C. by a thermostatically controlled lamp. Two hours after induction of ischemia, rats are reanesthetized, and the filament is withdrawn, as described in, e.g., Zhang et al., Stroke 27:317-323. Animals are then returned to their cages and closely monitored until recovery from anesthesia.

Under halothane anesthesia, the femoral artery is cannulated, and rats are placed on a stereotaxic frame. The arterial catheter is used for monitoring of arterial pressure and other parameters at different times after MCA occlusion. The MCA is occluded for 2 hours, as described above, and treatments are started, e.g., 6 hours after induction of ischemia. In one group of rats (e.g., 6), the combination therapy is administered, e.g., intraperitoneally, twice a day for 3 days. It should be noted that different doses, routes of administration, and times of administration can also be readily tested. A second group of rats is treated with a placebo administered in the same manner. Arterial pressure, rectal temperature, and plasma glucose are measured three times a day during the experiment. Arterial hematocrit and blood gases are measured before injection and 24, 48, and 72 hours after ischemia. Three days after MCA occlusion, brains are removed and frozen in cooled isopentane (−30° C.). Coronal forebrain sections (30 μM thick) are serially cut in cryostat, collected at 300 μm intervals, and stained with thionin for determination of infarct volume by an image analyzer (e.g., MCID, Imaging Research), as described in Iadecola et al., J Cereb Blood Flow Metab, 15:378-384, 1995. Infarct volume in cerebral cortex is corrected for swelling according to the method of Lin et al., Stroke 24:117-121, 1993, which is based on comparing the volumes of neocortex ipsilateral and contralateral to the stroke. The correction for swelling is needed to factor out the contribution of ischemic swelling to the total volume of the lesion (see Zhang and Iadecola, J Cereb Blood Flow Metab, 14:574-580, 1994). Reduction of infarct size in combination therapy-treated animals compared to animals receiving placebo is indicative of the efficacy of the combination therapy.

It should be noted that all of the above-mentioned procedures can be modified for a particular study, depending on factors such as a drug combination used, length of the study, subjects that are selected, etc. Such modifications can be designed by a skilled artisan without undue experimentation.

1. A method for treating a stroke, the method comprising:
(a) diagnosing a subject in need of treatment for a stroke; and
(b) administering to the subject a cyclooxygenase-2 selective inhibitor or an isomer, a pharmaceutically acceptable salt, ester, or prodrug thereof and a peroxisome proliferator activated receptor agonist or an isomer, a pharmaceutically acceptable salt, ester, or prodrug thereof.

2. The method of claim 1 wherein the cyclooxgenase-2 selective inhibitor has a selectivity ratio of COX-1 IC₅₀ to COX-2 IC₅₀ not less than about 50.

3. The method of claim 1 wherein the cyclooxgenase-2 selective inhibitor has a selectivity ratio of COX-1 IC₅₀ to COX-2 IC₅₀ not less than about 100.

4. The method of claim 1 wherein the cyclooxygenase-2 selective inhibitor is selected from the group consisting of celecoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, 2-[(2,4-dichloro-6-methylphenyl)amino]-5-ethyl-benzeneacetic acid, (3Z)-3-[(4-chlorophenyl)[4-(methylsulfonyl)phenyl]methylene]dihydro-2(3H)-furanone, and (S)-6,8-dichloro-2-(trifluoromethyl)-2H-1-benzopyran-3-carboxylic acid.

5. The method of claim 1 wherein the peroxisome proliferator activated receptor agonist is selected from the group consisting of ciglitazone, troglitazone, pioglitazone, englitazone, rosiglitazone, darglitazone clofibrate, fenofibrate, and bezafibrate or is an isomer, a pharmaceutically acceptable salt, ester, or prodrug thereof.

6. The method of claim 4 wherein the peroxisome proliferator activated receptor agonist is selected from the group consisting of ciglitazone, troglitazone, pioglitazone, englitazone, rosiglitazone, darglitazone clofibrate, fenofibrate, and bezafibrate or is an isomer, a pharmaceutically acceptable salt, ester, or prodrug thereof.

7. A method for treating a stroke, the method comprising:
(a) diagnosing a subject in need of treatment for a stroke; and
(b) administering to the subject a peroxisome proliferator activated receptor agonist or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a chromene compound, the chromene compound comprising a benzothiopyran, a dihydroquinoline or a dihydronaphthalene.

8. The method of claim 7 wherein the cyclooxgenase-2 selective inhibitor has a selectivity ratio of COX-1 IC₅₀ to COX-2 IC₅₀ not less than about 50.

9. The method of claim 7 wherein the cyclooxgenase-2 selective inhibitor has a selectivity ratio of COX-1 IC₅₀ to COX-2 IC₅₀ not less than about 100.

10. The method of claim 7 wherein the cyclooxygenase-2 selective inhibitor is a compound having the formula 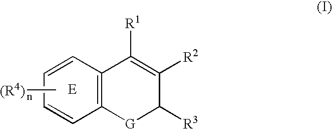
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or R⁴ together with the carbon atoms to which it is attached and the remainder of ring E forms a naphthyl radical.

11. The method of claim 7 wherein the cyclooxgyenase-2 selective inhibitor is (S)-6,8-dichloro-2-(trifluoromethyl)-2H-1-benzopyran-3-carboxylic acid.

12. The method of claim 7 wherein the peroxisome proliferator activated receptor agonist is selected from the group consisting of ciglitazone, troglitazone, pioglitazone, englitazone, rosiglitazone, darglitazone clofibrate, fenofibrate, and bezafibrate or is an isomer, a pharmaceutically acceptable salt, ester, or prodrug thereof.

13. A method for treating a stroke, the method comprising:
(a) diagnosing a subject in need of treatment for a stroke; and
(b) administering to the subject a peroxisome proliferator activated receptor agonist or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a tricyclic compound, the tricyclic compound containing a benzenesulfonamide or methylsulfonylbenzene moiety.

14. The method of claim 13 wherein the cyclooxgenase-2 selective inhibitor has a selectivity ratio of COX-1 IC₅₀ to COX-2 IC₅₀ not less than about 50.

15. The method of claim 13 wherein the cyclooxgenase-2 selective inhibitor has a selectivity ratio of COX-1 IC50 to COX-2 IC50 not less than about 100.

16. The method of claim 13 wherein the cyclooxygenase-2 selective inhibitor is a compound of the formula: 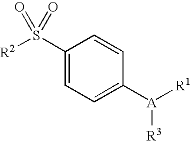
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl and amino; and
R³ is selected from the group consisting of H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, and N-alkyl-N-arylaminosulfonyl.

17. The method of claim 13 wherein the cyclooxygenase-2 selective inhibitor is selected from the group consisting of celecoxib, valdecoxib, parecoxib, deracoxib, rofecoxib, etoricoxib, and 2-(3,4-difluorophenyl)-4-(3-hydroxy-3-methylbutoxy)-5-[4-(methylsulfonyl)phenyl]-3(2H)-pyridazinone.

18. The method of claim 13 wherein the peroxisome proliferator activated receptor agonist is selected from the group consisting of ciglitazone, troglitazone, pioglitazone, englitazone, rosiglitazone, darglitazone clofibrate, fenofibrate, and bezafibrate or is an isomer, a pharmaceutically acceptable salt, ester, or prodrug thereof.

19. A method for treating a stroke, the method comprising:
(a) diagnosing a subject in need of treatment for a stroke; and
(b) administering to the subject a peroxisome proliferator activated receptor agonist or an isomer, a pharmaceutically acceptable salt, ester, or prodrug thereof and a cyclooxygenase-2 selective inhibitor or an isomer, a pharmaceutically acceptable salt, ester, or prodrug thereof, wherein the cyclooxygenase-2 selective inhibitor is a phenyl acetic acid compound.

20. The method of claim 19 wherein the cyclooxgenase-2 selective inhibitor has a selectivity ratio of COX-1 IC₅₀ to COX-2 IC₅₀ not less than about 50.

21. The method of claim 19 wherein the cyclooxgenase-2 selective inhibitor has a selectivity ratio of COX-1 IC₅₀ to COX-2 IC₅₀ not less than about 100.

22. The method of claim 19 wherein the cyclooxygenase-2 selective inhibitor is a compound having the formula: 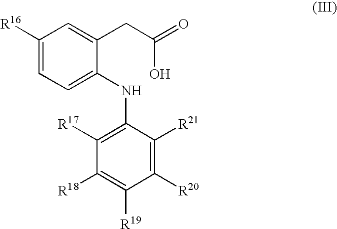
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl; and
provided, however, that each of R¹⁷, R¹⁸, R¹⁹ and R²⁰ is not fluoro when R¹⁶ is ethyl and R¹⁹ is H.

23. The method of claim 22
wherein:
R¹⁶ is ethyl;
R¹⁷ and R¹⁹ are chloro;
R¹⁸ and R²⁰ are hydrogen; and
R²¹ is methyl.

24. The method of claim 19 wherein the peroxisome proliferator activated receptor agonist is selected from the group consisting of ciglitazone, troglitazone, pioglitazone, englitazone, rosiglitazone, darglitazone clofibrate, fenofibrate, and bezafibrate or is an isomer, a pharmaceutically acceptable salt, ester, or prodrug thereof.

25. A method for treating a stroke, the method comprising:
(a) diagnosing a subject in need of treatment for a stroke; and
(b) administering to the subject a cyclooxygenase-2 selective inhibitor selected from the group consisting of celecoxib, deracoxib, valdecoxib, rofecoxib, lumiracoxib, etoricoxib, parecoxib, 2-(3,4-difluorophenyl)-4-(3-hydroxy-3-methylbutoxy)-5-[4-(methylsulfonyl)phenyl]-3(2H)-pyridazinone, and (S)-6,8-dichloro-2-(trifluoromethyl)-2H-1-benzopyran-3-carboxylic acid; and a peroxisome proliferator activated receptor agonist selected from the group consisting of ciglitazone, troglitazone, pioglitazone, englitazone, rosiglitazone, darglitazone clofibrate, fenofibrate, and bezafibrate or is an isomer, a pharmaceutically acceptable salt, ester, or prodrug thereof.

26. The method of claim 25 wherein the cyclooxygenase-2 selective inhibitor is celecoxib.

27. The method of claim 25 wherein the cyclooxygenase-2 selective inhibitor is deracoxib.

28. The method of claim 25 wherein the cyclooxygenase-2 selective inhibitor is valdecoxib.

29. The method of claim 25 wherein the cyclooxygenase-2 selective inhibitor is rofecoxib.

30. The method of claim 25 wherein the cyclooxygenase-2 selective inhibitor is etoricoxib.

31. The method of claim 25 wherein the cyclooxygenase-2 selective inhibitor is parecoxib.

32. The method of claim 25 wherein the cyclooxygenase-2 selective inhibitor is 2-(3,4-difluorophenyl)-4-(3-hydroxy-3-methylbutoxy)-5-[4-(methylsulfonyl)phenyl]-3(2H)-pyridazinone.

33. The method of claim 25 wherein the cyclooxygenase-2 selective inhibitor is (S)-6,8-dichloro-2-(trifluoromethyl)-2H-1-benzopyran-3-carboxylic acid.

34. The method of claim 25 wherein the cyclooxygenase-2 selective inhibitor is lumiracoxib.

35. The method of claim 1 wherein the stroke is a hemorrhagic stroke.

36. The method of claim 1 wherein the stroke is an ischemic stroke.
